(19) United States
(12) Patent Application Publication
(10) Pub. No.: US 2021/0093735 A1
(43) Pub. Date: Apr. 1, 2021

(54) METHODS AND COMPOSITIONS FOR TREATMENT OF HEMOPHILIA

(71) Applicant: The University of North Carolina at Chapel Hill, Chapel Hill, NC (US)

(72) Inventors: Chengwen Li, Chapel Hill, NC (US); Junjiang Sun, Chapel Hill, NC (US)

(21) Appl. No.: 17/050,561

(22) PCT Filed: Apr. 26, 2019
(86) PCT No.: PCT/US19/29374

§ 371 Date: Oct. 26, 2020

Related U.S. Application Data
(60) Provisional application No. 62/663,061, filed on Apr. 26, 2018.

(51) Int. Cl. A61K 48/00; C07K 14/745; C12N 15/86; A61P 7/04

(52) U.S. Cl. CPC A61K 48/0058; C07K 14/745; C12N 15/86; A61P 7/04; C12N 2750/14171; C12N 2750/14143; C12N 2750/14123; C12N 2740/15023; C12N 2740/15043; C07K 2319/02

(57) ABSTRACT

The present invention provides methods and compositions for treatment of hemophilia and other bleeding disorders in a subject in need thereof.

### STATEMENT OF PRIORITY

This application claims the benefit, under 35 U.S.C. § 119(e), of U.S. Provisional Application Ser. No. 62/663,061, filed Apr. 26, 2018, the entire contents of which are incorporated by reference herein.

### STATEMENT OF GOVERNMENT SUPPORT

This invention was made with government support under Grant Number HL144661 awarded by the National Institutes of Health. The government has certain rights in the invention.

### STATEMENT REGARDING ELECTRONIC FILING OF A SEQUENCE LISTING

A Sequence Listing in ASCII text format, submitted under 37 C.F.R. § 1.821, entitled 5470-835WO_ST25.txt, 62,997 bytes in size, generated on Apr. 26, 2019 and filed via EFS-Web, is provided in lieu of a paper copy. This Sequence Listing is incorporated by reference into the specification for its disclosures.

### FIELD OF THE INVENTION

This invention is directed to methods and compositions comprising an optimized factor Va (FVa) for treatment of hemophilia in a subject with or without an inhibitor.

### BACKGROUND OF THE INVENTION

Hemophilia is a bleeding disorder caused by the deficiency of coagulation factors in the contact activation pathway of the coagulation cascade. Protein replacement is currently the major treatment. The most severe complication in the treatment of hemophilia is the development of inhibitors to the infused clotting factors. After replacement therapy, about 30% of hemophilia A patients develop inhibitors to clotting factor VIII (FVIII) and/or −5% of hemophilia B patients develop inhibitors to clotting factor IX (FIX), which inhibits the efficiency of protein replacement. The treatment costs for patients with inhibitors are 3-5-fold higher than that for patients without inhibitors. Additionally, patients with inhibitors have more severe joint diseases and likelihood of hospitalization. Clotting factor VIIa (FVIIa), which is a bypass product in the coagulation cascade has been used in the treatment of patients with inhibitors. However, super-high doses of FVIIa and repeat infusions are needed to achieve a satisfactory therapeutic effect, which is a significant financial burden for patients. Gene therapy could ultimately provide a cure and obviate the need for repeated clotting factor infusions. Recently, gene therapy with adeno-associated virus (AAV) vectors to deliver FVIII or FIX has shown some beneficial effects; however, only to patients without inhibitors.

Thus, the present invention overcomes previous shortcomings in the art by providing compositions and methods of their use in the treatment of hemophilia in a subject with or without inhibitors.

### SUMMARY OF THE INVENTION

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

In one aspect, the present invention provides a synthetic protein molecule, comprising: a) a signal peptide; b) a factor Va (FVa) heavy chain (A1-A2 domains) comprising an amino acid sequence AQLRQFYVAAQGISWSYRPEPTNSSLNLSVTSFKKIVYREYEPYFKKEKPQSTISGLL GPTLYAEVGDIIKVHFKNKADKPLSIHPQGIRYSKLSEGASYLDHTFPAEKMDDAVAP GREYTYEWSISEDSGPTHDDPPCLTHIYYSHENLIEDFNSGLIGPLLICKKGTLTEGGT QKTFDKQIVLLFAVFDESKSWSQSSSLMYTVNGYVNGTMPDITVCAHDHISWHLLG MSSGPELFSIHFNGQVLEQNHHKVSAITLVSATSTTANMTVGPEGKWIISSLTPKHLQ AGMQAYIDIKNCPKKTRNLKKITREQRRHMKRWEYFIAAEEVIWDYAPVIPANMDK KYRSQHLDNFSNQIGKHYKKVMYTQYEDESFTKHTVNPNMKEDGILGPIIRAQVRDT LKIVFKNMASRPYSIYPHGVTFSPYEDEVNSSFTSGRNNTMIRAVQPGETYTYKWNIL EFDEPTENDAQCLTRPYYSDVDIMRDIASGLIGLLLICKSRSLDRRGIQRAADIEQQAV FAVFDENKSWYLEDNINKFCENPDEVKRDDPKFYESNIMSTINGYVPESITTLGFCFD DTVQWHFCSVGTQNEILTIHFTGHSFIYGKRHEDTLTLFPMRGESVTVTMDNVGTW MLTSMNSSPRSKKLRLKFRDVKCIPDDDEDSYEIFEPPESTVMATRKMHDRLEPEDEE SDADYDYQNRLAAALGIR (SEQ ID NO: 2); c) a linker sequence; and d) a FVa light chain (A3-C1-C2 domains) comprising an amino acid sequence SNNGNRRNYYIAAEEISWDYSEFVQRETDIEDSDDIPEDTTYKKVVFRKYLDSTFTKR DPRGEYEEHLGILGPIIRAEVDDVIQVRFKNLASRPYSLHAHGLSYEKSSEGKTYEDD SPEWFKEDNAVQPNSSYTYVWHATERSGPESPGSACRAWAYYSAVNPEKDIHSGLI GPLLICQKGILHKDSNMPMDMREFVLLFMTFDEKKSWYYEKKSRSSWRLTSSEMKK SHEFHAINGMIYSLPGLKMYEQEWVRLHLLNIGGSQDIHVVHFHGQTLLENGNKQH QLGVWPLLPGSFKTLEMKASKPGWWLLNTEVGENQRAGMQTPFLIMDRDCRMPM GLSTGIISDSQIKASEFLGYWEPRLARLNNGGSYNAWSVEKLAAEFASKPWIQVDMQ KEVIITGIQTQGAKHYLKSCYTTEFYVAYSSNQINWQIFKGNSTRNVMYFNGNSDAS TIKENQFDPPIVARYIRISPTRAYNRPTLRLELQGCEVNGCSTPLGMENGKIENKQITA SSFKKSWWGDYWEPFRARLNAQGRVNAWQAKANNNKQWLEIDLLKIKKITAIITQG CKSLSSEMYVKSYTIHYSEQGVEWKPYRLKSSMVDKIFEGNTNTKGHVKNFFNPPIIS RFIRVIPKTWNQSIALRLELFGCDIY (SEQ ID NO: 3), with the proviso that the recombinant protein molecule does not include all or part of a FVa B domain.

The amino acid sequence of a human FvB domain is:

(SEQ ID NO: 4)
SFRNSSLNQEEEEFNLTALALENGTEFVSSNTDIIVGSNYSSPSNISK
FTVNNLAEPQKAPSHQQATTAGSPLRHLIGKNSVLNSSTAEHSSPYSE
DPIEDPLQPDVTGIRLLSLGAGEFKSQEHAKHKGPKVERDQAAKHRFS
WMKLLAHKVGRHLSQDTGSPSGMRPWEDLPSQDTGSPSRMRPWKDPPS
DLLLLKQSNSSKILVGRWHLASEKGSYEIIQDTDEDTAVNNWLISPQN
ASRAWGESTPLANKPGKQSGHPKFPRVRHKSLQVRQDGGKSRLKKSQF
LIKTRKKKKEKHTHHAPLSPRTFHPLRSEAYNTFSERRLKHSLVLHKS
NETSLPTDLNQTLPSMDFGWIASLPDHNQNSSNDTGQASCPPGLYQTV
PPEEHYQTFPIQDPDQMHSTSDPSHRSSSPELSEMLEYDRSHKSFPTD
ISQMSPSSEHEVWQTVISPDLSQVTLSPELSQTNLSPDLSHTTLSPEL
IQRNLSPALGQMPISPDLSHTTLSPDLSHTTLSLDLSQTNLSPELSQT
NLSPALGQMPLSPDLSHTTLSLDFSQTNLSPELSHMTLSPELSQTNLS
PALGQMPISPDLSHTTLSLDFSQTNLSPELSQTNLSPALGQMPLSPDP
SHTTLSLDLSQTNLSPELSQTNLSPDLSEMPLFADLSQIPLTPDLDQM
TLSPDLGETDLSPNFGQMSLSPDLSQVTLSPDISDTTLLPDLSQISPP
PDLDQIFYPSESSQSLLLQEFNESFPYPDLGQMPSPSSPTLNDTFLSK
EFNPLVIVGLSKDGTDYIEIIPKEEVQSSEDDYAEIDYVPYDDPYKTD
VRTNINSSRDPDNIAAWYLR.

In a further aspect, the present invention provides a nucleic acid molecule comprising a nucleotide sequence that encodes the synthetic protein molecule of this invention.

In another aspect, the present invention provides a recombinant nucleic acid construct comprising the nucleic acid molecule of this invention.

In another aspect, the present invention provides an AAV particle comprising the nucleic acid molecule of this invention, the recombinant nucleic acid construct of this invention, or the recombinant nucleic acid molecule of this invention.

In another aspect, the invention provides a composition comprising the synthetic protein molecule, any of the nucleic acid molecules and/or an AAV particle of this invention in a pharmaceutically acceptable carrier.

In another aspect, the invention provides a method of administering a nucleic acid molecule to a cell, the method comprising contacting the cell with a nucleic acid molecule, a recombinant nucleic acid construct, and/or an AAV particle of this invention, and/or any composition of this invention.

In another aspect, the invention provides a method of delivering a nucleic acid molecule to a subject, the method comprising administering to the subject the AAV particle of this invention or the composition of this invention. In some embodiments, the subject has a bleeding disorder or disease. For example, in some embodiments, the subjects has a deficiency in a clotting factor, e.g., clotting factor(s) II, V, VII, VIII, IX, X, XI, or XII resulting in bleeding disorders and/or abnormal bleeding problems. In some embodiments, the subject has experienced extensive tissue damage in association with surgery or trauma. In another aspect, the invention provides a method of treating a bleeding disorder in a subject (e.g., a subject in need thereof) comprising administering to the subject a nucleic acid molecule, a recombinant nucleic acid construct, and/or an AAV particle of this invention, and/or any composition of this invention.

### BRIEF DESCRIPTION OF THE DRAWINGS

FIG. 1. Diagram of hFV constructs. CMV.hFV: wild type of human factor V driven by the CMV promoter. TTR.BD.furing: hFV with complete deletion of B domain and a furin cleavage site linker between the FV heavy chain (HC) and the light chain (LC). TTR.BD.SQ: hFVa with small B domain remaining TTR.BD.4119: hFV with large B domain remaining TTR.hFV.BD: hFV with complete deletion of B domain.

FIG. 2. Functional analysis of different hFVa constructs. Plasmids from FIG. 1 were administered into hemophilia B mice via hydrodynamic injection. Two days later, blood was collected for aPPT analysis. The data represented the average and standard derivation of 4 mice.

FIG. 3. Detection of FVa from transfection of pCBA-FVa. pCBA-hFVa was transfected in 293 cells, 3 days later; supernatant was harvested for the FVa heavy chain (HC) detection. Lane 1: hFVa, lane 2: Green Fluorescent Protein (GFP).

FIG. 4. Complete phenotypic correction after administration of AAV8/FVa-furin in hemophilia mice. 1×10¹² particles of AAV8/hFVa were administered into hemophilia. B mice via tail vein. Blood was harvested for coagulation assay. The data represented the average and standard derivation of 4 mice.

FIG. 5. Improved phenotypic correction with AAV8/FVa-opt. 3×10×¹¹ particles of AAV8/hFVa or AAV8/hFVa-opt were administered into hemophilia B mice via tail vein. At week 1 and 4 post AAV injection, blood was harvested for coagulation assay. The data represented the average and standard derivation of 4 mice.

FIG. 6. Diagram of hFVa cassettes.

FIG. 7. The effects of different promoters on FVa function in HB mice. 1×10×¹¹ particles of AAV8/hFVa-opt driven by different promoters were administered into hemophilia B mice via tail vein. At pre and week 8 post AAV injections, blood was harvested for coagulation assay. The percentage of clot time change for APTT at week 8 post AAV administrations was calculated while compared to APTT time pre-AAV injection. The data represented the average and standard derivation of 4 mice.

FIG. 8. Transduction in Huh7 cell with different promoters. AAV8/luc vectors encoding firefly transgene driven by different promoters at a dose of 1×10⁴ particles/cell were used to infect Huh7 cells. Two days after AAV transduction, cell lysate was harvested for luciferase assay.

FIG. 9. Phenotypic correction in hemophilia A mice with inhibitors after systemic administration of AAV/hFVa. Hemophilia A mice were treated with recombinant FVIII for inhibitor development. 2×10¹² particles of AAV8/TTR-hFVA were administered via retro-orbital injection. At weeks 1 and 2, blood was collected for aPTT assay. Mice without rhFVIII immunization served as control. The data represented the average and standard derivation of 5 mice.

### DETAILED DESCRIPTION OF THE INVENTION

The present invention will now be described more fully hereinafter with reference to the accompanying drawings and specification, in which preferred embodiments of the invention are shown. This invention may, however, be embodied in different forms and should not be construed as limited to the embodiments set forth herein.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

Nucleotide sequences are presented herein by single strand only, in the 5′ to 3′ direction, from left to right, unless specifically indicated otherwise. Nucleotides and amino acids are represented herein in the manner recommended by the IUPAC-IUB Biochemical Nomenclature Commission, or (for amino acids) by either the one-letter code, or the three letter code, both in accordance with 37 C.F.R. § 1.822 and established usage.

Except as otherwise indicated, standard methods known to those skilled in the art may be used for cloning genes, amplifying and detecting nucleic acids, and the like. Such techniques are known to those skilled in the art. See, e.g., Sambrook et al., Molecular Cloning: A Laboratory Manual 2nd Ed. (Cold Spring Harbor, N.Y., 1989); Ausubel et al. Current Protocols in Molecular Biology (Green Publishing Associates, Inc. and John Wiley & Sons, Inc., New York).

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

All publications, patent applications, patents and other references cited herein are incorporated by reference in their entireties for the teachings relevant to the sentence and/or paragraph in which the reference is presented.

The invention, in part, relates to methods of using a synthetic protein molecule in the treatment of bleeding disorders. Bleeding disorders are a group of conditions that result when the blood cannot clot properly. Such a condition may be genetic (i.e., inherited from a family member) or acquired (e.g., autoimmune disorders; drug treatment, etc.).

In normal clotting (also known as coagulation), platelets, a type of blood cell, stick together and form a plug at the site of an injured blood vessel. Proteins in the blood called clotting factors then interact to form a fibrin clot, essentially a gel plug, which holds the platelets in place and allows healing to occur at the site of the injury while preventing blood from escaping the blood vessel. Typically, in bleeding disorders a deficiency of at least one clotting factor required for clotting is present. For example, deficiencies in clotting factor(s) II, V, VII, X, XI, or XII result in bleeding disorders and/or abnormal bleeding problems. Hemophilia is another example of a bleeding disorder and is classified as type A or type B, based on which type of clotting factor is deficient (factor VIII in type A and factor IX in type B).

As mentioned already, one possible treatment option for subjects suffering from bleeding disorders, such as Hemophilia A (HA) and Hemophilia B (HB) is protein replacement therapy. Clotting factors are replaced by injecting (infusing) a clotting factor concentrate into a vein to help blood to clot normally. For example, clotting factor VIIa has been used to control bleeding disorders by stimulating the coagulation cascade in a subject. In some embodiments, the subject has a normal functioning clotting cascade (i.e., no clotting factor deficiencies) and requires control of excessive bleeding caused by defective platelet function, thrombocytopenia, von Willebrand disease, surgery, and other forms of trauma.

Unfortunately, some subjects develop neutralizing inhibitors against the infused clotting factors, which leaves the subject unaffected by the factor treatment. The inhibitor (i.e., antibody and/or other immune component) forms because the body stops accepting the factor treatment product as a normal part of the blood and recognizes the factor as a foreign substance. The inhibitor(s) can appear and disappear anytime during the treatment course.

To avoid the formation of inhibitors, alternate treatment options targeting bypassing agents in the coagulation cascade are being considered. Examples of alternate bypass agents include, but are not be limited to, activated clotting factor VII (FVIIa), including recombinant human (rh) FVIIa, and plasma-derived activated prothrombin complex concentrates. The current invention relates to methods and compositions comprising activated clotting factor V (FVa), which is another alternate bypass agent. FVa is a cofactor that binds to FXa during the formation of the prothrombinase complex, which activates prothrombin to thrombin. FVa is able to enhance the rate of thrombin generation by approximately 10,000 fold. Thrombin plays an important role in the coagulation cascade, e.g., it promotes platelet activation and aggregation and it converts FXI to FXIa, VIII to VIIIa, V to Va, fibrinogen to fibrin, and XIII to XIIIa.

The current invention also relates to methods and compositions comprising a combination of bypass agents, such as FVIIa and FVa and any variant and/or derivative thereof. Not to be bound by theory, it is believed that because FVII and FVa have different mechanisms for generating thrombin, this particular combination of bypassing agents (FVIIa and FVa and/or any variant and/or derivative thereof) exhibits beneficial and/or synergistic therapeutic effects in the treatment of a subject (e.g., with inhibitors) that has a bleeding disorder.

FVa (or any variant and/or derivative thereof) alone or in combination with FVIIa (or any variant and/or derivative thereof) can be administered to a subject in need thereof using any known method in the art, e.g., using a viral vector such as adeno-associated virus (AAV), retrovirus, lentivirus, poxvirus, alphavirus, baculovirus, vaccinia virus, herpes virus, and Epstein-Barr virus.

AAV is a small (25-nm), nonenveloped virus that packages a linear single-stranded DNA genome. AAV can infect both dividing and quiescent cells and persist in an extrachromosomal state without integrating into the genome of the host cell, although in the native virus some integration of virally carried genes into the host genome does occur. However, due to the size limitation of the AAV virion package (i.e., less than 4.7 kb), deletion of some or all of the coding sequences for the B-domain in the full-length human FVa cDNA facilitates efficient delivery and/or expression of the nucleic acid molecule encoding FVa.

Thus, in some embodiments, the current invention provides 1 a synthetic protein molecule, comprising: a) a signal peptide; b) a factor Va (FVa) heavy chain (A1-A2 domains) comprising the amino acid sequence AQLRQFYVAAQGISWSYRPEPTNSSLNLSVTSFKKIVYREYEPYFKKEKPQSTISGLL GPTLYAEVGDIIKVHFKNKADKPLSIHPQGIRYSKLSEGASYLDHTFPAEKMDDAVAP GREYTYEWSISEDSGPTHDDPPCLTHIYYSHENLIEDFNSGLIGPLLICKKGTLTEGGT QKTFDKQIVLLFAVFDESKSWSQSSSLMYTVNGYVNGTMPDITVCAHDHISWHLLG MSSGPELFSIHFNGQVLEQNHHKVSAITLVSATSTTANMTVGPEGKWIISSLTPKHLQ AGMQAYIDIKNCPKKTRNLKKITREQRRHMKRWEYFIAAEEVIWDYAPVIPANMDK KYRSQHLDNFSNQIGKHYKKVMYTQYEDESFTKHTVNPNMKEDGILGPIIRAQVRDT LKIVFKNMASRPYSIYPHGVTFSPYEDEVNSSFTSGRNNTMIRAVQPGETYTYKWNIL EFDEPTENDAQCLTRPYYSDVDIMRDIASGLIGLLLICKSRSLDRRGIQRAADIEQQAV FAVFDENKSWYLEDNINKFCENPDEVKRDDPKFYESNIMSTINGYVPESITTLGFCFD DTVQWHFCSVGTQNEILTIHFTGHSFIYGKRHEDTLTLFPMRGESVTVTMDNVGTW MLTSMNSSPRSKKLRLKFRDVKCIPDDDEDSYEIFEPPESTVMATRKMHDRLEPEDEE SDADYDYQNRLAAALGIR (SEQ ID NO: 2); c) a linker sequence; and d) a FVa light chain (A3-C1-C2 domains) comprising the amino acid sequence SNNGNRRNYYIAAEEISWDYSEFVQRETDIEDSDDIPEDTTYKKVVFRKYLDSTFTKR DPRGEYEEHLGILGPIIRAEVDDVIQVRFKNLASRPYSLHAHGLSYEKSSEGKTYEDD SPEWFKEDNAVQPNSSYTYVWHATERSGPESPGSACRAWAYYSAVNPEKDIHSGLI GPLLICQKGILHKDSNMPMDMREFVLLFMTFDEKKSWYYEKKSRSSWRLTSSEMKK SHEFHAINGMIYSLPGLKMYEQEWVRLHLLNIGGSQDIHVVHFHGQTLLENGNKQH QLGVWPLLPGSFKTLEMKASKPGWWLLNTEVGENQRAGMQTPFLIMDRDCRMPM GLSTGIISDSQIKASEFLGYWEPRLARLNNGGSYNAWSVEKLAAEFASKPWIQVDMQ KEVIITGIQTQGAKHYLKSCYTTEFYVAYSSNQINWQIFKGNSTRNVMYFNGNSDAS TIKENQFDPPIVARYIRISPTRAYNRPTLRLELQGCEVNGCSTPLGMENGKIENKQITA SSFKKSWWGDYWEPFRARLNAQGRVNAWQAKANNNKQWLEIDLLKIKKITAIITQG CKSLSSEMYVKSYTIHYSEQGVEWKPYRLKSSMVDKIFEGNTNTKGHVKNFFNPPIIS RFIRVIPKTWNQSIALRLELFGCDIY (SEQ ID NO: 3), with the proviso that the recombinant protein molecule does not include a FVa B domain.

In some embodiments, the signal peptide of the synthetic protein molecule this invention can comprise an amino acid sequence which can be, but is not limited to: MFPGCPRLWVLVVLGTSWVGWGSQGTEA (SEQ ID NO:1); hFVII: MVSQALRLLCLLLGLQGCLA (SEQ ID NO:6); hFIX: MQRVNMIMAESPGLITICLLGYLLSAEC (SEQ ID NO:7); hFVIII: MQIELSTCFFLCLLRFCFS (SEQ ID NO:8); Human fibrinogen-alpha chain: MFSMRIVCLVLSVVGTAWT (SEQ ID NO:9); Human fibrinogen-beta chain: MKRMVSWSFHKLKTMKHLLLLLLCVFLVKS (SEQ ID NO:10); Human fibrinogen-gamma chain: MSWSLHPRNLILYFYALLFLSSTCVA (SEQ ID NO:11); hFXII: MRALLLLGFLLVSLESTLS (SEQ ID NO:12); Protein C: MWQLTSLLLFVATWGISG (SEQ ID NO:13); Protein S: MRVLGGRCGALLACLLLVLPVSEA (SEQ ID NO:14); Thrombin: MAHVRGLQLPGCLALAALCSLVHS (SEQ ID NO:15); Anti-thrombin: MYSNVIGTVTSGKRKVYLLSLLLIGFWDCVTC (SEQ ID NO:16); Serum albumin: MKWVTFISLLFLFSSAYS (SEQ ID NO:17); Transferrin: MRLAVGALLVCAVLGLCLA (SEQ ID NO:18); Alpha-1 antitrypsin: MPSSVSWGILLLAGLCCLVPVSLA (SEQ ID NO:19); Fibronectin: MLRGPGPGLLLLAVQCLGTAVPSTGASKSKR (SEQ ID NO:20); Alpha-1-microglobulin: MRSLGALLLLLSACLAVSA (SEQ ID NO:21); Alpha 1-antichymotrypsin: MERMLPLLALGLLAAGFCPAVLC (SEQ ID NO:22); Apo A: MKAAVLTLAVLFLTGSQA (SEQ ID NO:23); Apo B: MDPPRPALLALLALPALLLLLLAGARA (SEQ ID NO:24); Apo E: MKVLWAALLVTFLAGCQA (SEQ ID NO:25); Alpha-fetoprotein: MKWVESIFLIFLLNFTES (SEQ ID NO:26); C-reactive protein: MEKLLCFLVLTSLSHAFG (SEQ ID NO:27); Plasminogen: MEHKEVVLLLLLFLKSGQG (SEQ ID NO:28); Ceruloplasmin: MKILILGIFLFLCSTPAWA (SEQ ID NO:29); Complement C1q subunit A: MEGPRGWLVLCVLAISLASMVT (SEQ ID NO:30); Complement C2: MGPLMVLFCLLFLYPGLADS (SEQ ID NO:31); Complement C3: MGPTSGPSLLLLLLTHLPLALG (SEQ ID NO:32); Complement C4A: MRLLWGLIWASSFFTLSLQ (SEQ ID NO:33); Complement C5: MGLLGILCFLIFLGKTWG (SEQ ID NO:34); Complement C6: MARRSVLYFILLNALINKGQA (SEQ ID NO:35); Complement C7: MKVISLFILVGFIGEFQSFSSA (SEQ ID NO:36); Complement C8A: MFAVVFFILSLMTCQPGVTA (SEQ ID NO:37); Complement C9: MSACRSFAVAICILEISILTA (SEQ ID NO:38); α2-antiplasmin: MALLWGLLVLSWSCLQGPCSVFSPVSA (SEQ ID NO:39); Transcortin: MPLLLYTCLLWLPTSGLWTVQA (SEQ ID NO:40); Haptoglobin: MSALGAVIALLLWGQLFA (SEQ ID NO:41); Hemopexin: MARVLGAPVALGLWSLCWSLAIA (SEQ ID NO:42); IGF binding protein 1: MSEVPVARVWLVLLLLTVQVGVTAG (IGFBP2-7) (SEQ ID NO:43); Transthyretin: MASHRLLLLCLAGLVFVSEA (SEQ ID NO:44); Insulin-like growth factor 1 (IGF-1): MGKISSLPTQLFKCCFCDFLK (SEQ ID NO:45); Thrombopoietin: MELTELLLVVMLLLTARLTLS (SEQ ID NO:46); 132 microglobulin: MSRSVALAVLALLSLSGLEA (SEQ ID NO:47); alpha-2-Macroglobulin: MGKNKLLHPSLVLLLLVLLPTDA (SEQ ID NO:48); and any other signal peptides now known or later identified. The signal peptide in this invention can be present singly or in multiples and/or in any combination with signal peptides.

In some embodiments, the linker sequence of the synthetic protein molecule of this invention comprises an amino acid sequence which can be a furin cleavage motif (RKRRKR) (SEQ ID NO:49); a 2A peptide, a protein linker comprising the formulae (GGGGS)ₙ, (GS)ₙ; any length of snake FV B domain; any length of human FV B domain N-terminus within 100 aa; any length of human FV B domain C-terminus within 100 aa; any length of human FVIII B domain N-terminus within 100 aa; any length of human FVIII B domain C-terminus within 100 aa; and combinations thereof.

In some embodiments, the invention provides a nucleic acid molecule comprising a nucleotide sequence that encodes the synthetic protein molecule of this invention. In some embodiments, the nucleic acid molecule of this invention comprises a nucleotide sequence that has been optimized to increase expression of the nucleotide sequence relative to a nucleotide sequence that has not been optimized.

In some embodiments, the nucleic acid molecule of this invention further comprises a promoter sequence. In some embodiments, the promoter sequence of the nucleic acid molecule can be TTR (transthyretin); TTR/mvm (TTR promoter with Minute Virus of Mice (MVM) intron); HLP (human liver specific promoter; 251-bp fragment containing a 34-bp core enhancer from the human apolipoprotein hepatic control region; modified 217-bp α-1-antitrypsin (AIAT) promoter); Ch19-AIAT (122 bp from AAV integrated site from chromosome 19 and 185 bp of AIAT promoter, one or more than one copy of Ch19 fragment, in different orientations; pHU1-1 (a minimal human 243 bp cellular small nuclear RNA promoter); the human elongation factor 1-alpha promoter; herpes simplex thymidine kinase (Tk) promoter (pDLZ2); Tk promoter linked to enhancer I of hepatitis B virus; a synthetic, basic albumin promoter; a synthetically derived short liver-specific promoter/enhancer of 368 bp from the insulin-like growth factor-binding protein followed by a 175-bp chimeric intron (IGBP/enh/intron); beta-actin minimum promoter; a cytomegalovirus promoter (CMV); a human β-actin promoter with a CMV enhancer (CB); liver-specific human alpha1 anti-trypsin promoter (HAAT) and the liver-specific hepatic control region (HCR) enhancer/human alpha1 anti-trypsin promoter complex (HCRHAAT); human insulin-like growth factor binding protein (IGFBP) promoter; HCR-hAAT (the human apolipoprotein E/C-I gene locus control region (HCR) and the human α1 antitrypsin promoter (hAAT) with a chicken β actin/rabbit β globin composite intron); U1a1 small nuclear RNA promoter; histone H2 promoter; U1b2 small nuclear RNA promoter; histone H3 promoter; α-antitrypsin promoter; human factor IX promoter with liver transcription factor-responsive oligomers; CM1 promoter (HCR/ApoE enhancer/α-antitrypsin promoter); LSP (liver specific promoter: TH-binding globulin promoter/α1-microglobulin/bikunin enhancer); or any other promoter now known or alter developed. The promoter of this invention can be present singly or in multiples and/or any combination with other promoters.

In further embodiments, the present invention provides a synthetic promoter comprising, consisting essentially of and/or consisting of the nucleotide sequence: tctggcgatttccactgggcgcctcggagctgcggacttcccagtgtgcatcggggcacagcgactcctggaagtggccaagggcc acttctgctaatggactccatttcccagcgctcccc (SEQ ID NO:54), operably linked to the nucleotide sequence: ggcgactcagatcccagccagtggacttagcccctgtttgctcctccgataactggggtgaccttggttaatattcaccagcagcctccc ccgttgcccctctggatccactgcttaaatacggacgaggacagggccctgtctcctcagcttcaggcaccaccactgacctgggaca gtgaatc (SEQ ID NO:55). The respective nucleotide sequences can be linked via a nucleotide linker that can comprise, consist essentially of and/or consist of about 1, 2, 3, 4, 5, 6, 7, 8, 9, 10, 15, 20, 25, 30, 35, 40, 50, 60, 70, 80, 90, 100, etc. nucleotides that operably link the respective nucleotide sequences.

The present invention also provides a synthetic promoter sequence, comprising, consisting essentially of, and/or consisting of the nucleotide sequence:

(SEQ ID NO: 56)
tctggcgatttccactgggcgcctcggagctgcggacttcccagtgtg
catcggggcacagcgactcctggaagtggccaagggccacttctgcta
atggactccatttcccagcgctccccagatctgggcgactcagatccc
agccagtggacttagcccctgtttgctcctccgataactggggtgacc
ttggttaatattcaccagcagcctcccccgttgcccctctggatccac
tgcttaaatacggacgaggacagggccctgtctcctcagcttcaggca
ccaccactgacctgggacagtgaatc.

The synthetic promoter of this invention, having the nucleotide sequence of SEQ ID NO:54 linked to the nucleotide sequence of SEQ ID NO:55, and/or the promoter of this invention, having the nucleotide sequence of SEQ ID NO:56, can be included in any of the nucleic acid molecules, recombinant nucleic acid constructs and/or virus particles of this invention.

In some embodiments, the invention provides a recombinant nucleic acid construct comprising the nucleic acid molecule of this invention.

In some embodiments, the invention provides a recombinant nucleic acid molecule, comprising an adeno-associated virus (AAV) 5′ inverted terminal repeat (ITR) and the nucleic acid molecule of this invention operably linked to a promoter and an AAV 3′ ITR.

In some embodiments, the invention provides an AAV particle comprising the nucleic acid molecule, the recombinant nucleic acid construct, or the recombinant nucleic acid molecule of this invention.

In some embodiments, the invention provides a recombinant nucleic acid molecule, comprising a lentivirus 5′ long terminal repeat (LTR) and the nucleic acid molecule of this invention operably linked to a promoter and a lentivirus 3′ LTR.

In some embodiments, the invention provides a lentivirus particle comprising the nucleic acid molecule of this invention, the recombinant nucleic acid construct, or the recombinant nucleic acid molecule of this invention.

In some embodiments, the invention provides a recombinant nucleic acid molecule comprising an adenovirus (Ad) 5′ ITR and the nucleic acid molecule of this invention operably linked to a promoter and an AAV 3′ ITR.

In some embodiments, the invention provides an Ad particle comprising the nucleic acid molecule, the recombinant nucleic acid construct, or the recombinant nucleic acid molecule of this invention.

In some embodiments, the invention provides a plasmid comprising the nucleic acid molecule and/or the recombinant nucleic acid construct of this invention. In some embodiments, the plasmid has one or more selected marker genes.

In some embodiments, the invention provides a recombinant nucleic acid molecule encoding the hFV protein with whole B-domain deletion comprising the nucleotide sequence:

(SEQ ID NO: 50)
atgttcccaggctgcccacgcctctgggtcctggtggtcttgggcacc
agctgggtaggctgggggagccaagggacagaagcggcacagctaagg
cagttctacgtggctgctcagggcatcagttggagctaccgacctgag
cccacaaactcaagtttgaatctttctgtaacttcctttaagaaaatt
gtctacagagagtatgaaccatattttaagaaagaaaaaccacaatct
accatttcaggacttcttgggcctactttatatgctgaagtcggagac
atcataaaagttcactttaaaaataaggcagataagcccttgagcatc
catcctcaaggaattaggtacagtaaattatcagaaggtgcttcttac
cttgaccacacattccctgcggagaagatggacgacgctgtggctcca
ggccgagaatacacctatgaatggagtatcagtgaggacagtggaccc
acccatgatgaccctccatgcctcacacacatctattactcccatgaa
aatctgatcgaggatttcaactcggggctgattgggcccctgcttatc
tgtaaaaaagggaccctaactgagggtgggacacagaagacgtttgac
aagcaaatcgtgctactatttgctgtgtttgatgaaagcaagagctgg
agccagtcatcatccctaatgtacacagtcaatggatatgtgaatggg
acaatgccagatataacagtttgtgcccatgaccacatcagctggcat
ctgctgggaatgagctcggggccagaattattctccattcatttcaac
ggccaggtcctggagcagaaccatcataaggtctcagccatcaccctt
gtcagtgctacatccactaccgcaaatatgactgtgggcccagaggga
aagtggatcatatcttctctcaccccaaaacatttgcaagctgggatg
caggcttacattgacattaaaaactgcccaaagaaaaccaggaatctt
aagaaaataactcgtgagcagaggcggcacatgaagaggtgggaatac
ttcattgctgcagaggaagtcatttgggactatgcacctgtaatacca
gcgaatatggacaaaaaatacaggtctcagcatttggataatttctca
aaccaaattggaaaacattataagaaagttatgtacacacagtacgaa
gatgagtccttcaccaaacatacagtgaatcccaatatgaaagaagat
gggattttgggtcctattatcagagcccaggtcagagacacactcaaa
atcgtgttcaaaaatatggccagccgcccctatagcatttaccctcat
ggagtgaccttctcgccttatgaagatgaagtcaactcttctttcacc
tcaggcaggaacaacaccatgatcagagcagttcaaccaggggaaacc
tatacttataagtggaacatcttagagtttgatgaacccacagaaaat
gatgcccagtgcttaacaagaccatactacagtgacgtggacatcatg
agagacatcgcctctgggctaataggactacttctaatctgtaagagc
agatccctggacaggcgaggaatacagagggcagcagacatcgaacag
caggctgtgtttgctgtgtttgatgagaacaaaagctggtaccttgag
gacaacatcaacaagttttgtgaaaatcctgatgaggtgaaacgtgat
gaccccaagttttatgaatcaaacatcatgagcactatcaatggctat
gtgcctgagagcataactactcttggattctgctttgatgacactgtc
cagtggcacttctgtagtgtggggacccagaatgaaattttgaccatc
cacttcactgggcactcattcatctatggaaagaggcatgaggacacc
ttgaccctcttccccatgcgtggagaatctgtgacggtcacaatggat
aatgttggaacttggatgttaacttccatgaattctagtccaagaagc
aaaaagctgaggctgaaattcagggatgttaaatgtatcccagatgat
gatgaagactcatatgagatttttgaacctccagaatctacagtcatg
gctacacggaaaatgcatgatcgtttagaacctgaagatgaagagagt
gatgctgactatgattaccagaacagactggctgcagcattaggaatc
aggagaaagagaagaaagagaagcaacaatggaaacagaagaaattat
tacattgctgctgaagaaatatcctgggattattcagaatttgtacaa
agggaaacagatattgaagactctgatgatattccagaagataccaca
tataagaaagtagtttttcgaaagtacctcgacagcacttttaccaaa
cgtgatcctcgaggggagtatgaagagcatctcggaattcttggtcct
attatcagagctgaagtggatgatgttatccaagttcgttttaaaaat
ttagcatccagaccgtattctctacatgcccatggactttcctatgaa
aaatcatcagagggaaagacttatgaagatgactctcctgaatggttt
aaggaagataatgctgttcagccaaatagcagttatacctacgtatgg
catgccactgagcgatcagggccagaaagtcctggctctgcctgtcgg
gcttgggcctactactcagctgtgaacccagaaaaagatattcactca
ggcttgataggtcccctcctaatctgccaaaaaggaatactacataag
gacagcaacatgcctatggacatgagagaatttgtcttactatttatg
acctttgatgaaaagaagagctggtactatgaaaagaagtcccgaagt
tcttggagactcacatcctcagaaatgaaaaaatcccatgagtttcac
gccattaatgggatgatctacagcttgcctggcctgaaaatgtatgag
caagagtgggtgaggttacacctgctgaacataggcggctcccaagac
attcacgtggttcactttcacggccagaccttgctggaaaatggcaat
aaacagcaccagttaggggtctggccccttctgcctggttcatttaaa
actcttgaaatgaaggcatcaaaacctggctggtggctcctaaacaca
gaggttggagaaaaccagagagcagggatgcaaacgccatttcttatc
atggacagagactgtaggatgccaatgggactaagcactggtatcata
tctgattcacagatcaaggcttcagagtttctgggttactgggagccc
agattagcaagattaaacaatggtggatcttataatgcttggagtgta
gaaaaacttgcagcagaatttgcctctaaaccttggatccaggtggac
atgcaaaaggaagtcataatcacagggatccagacccaaggtgccaaa
cactacctgaagtcctgctataccacagagttctatgtagcttacagt
tccaaccagatcaactggcagatcttcaaagggaacagcacaaggaat
gtgatgtattttaatggcaattcagatgcctctacaataaaagagaat
cagtttgacccacctattgtggctagatatattaggatctctccaact
cgagcctataacagacctacccttcgattggaactgcaaggttgtgag
gtaaatggatgttccacacccctgggtatggaaaatggaaagatagaa
aacaagcaaatcacagcttcttcgtttaagaaatcttggtggggagat
tactgggaacccttccgtgcccgtctgaatgcccagggacgtgtgaat
gcctggcaagccaaggcaaacaacaataagcagtggctagaaattgat
ctactcaagatcaagaagataacggcaattataacacagggctgcaag
tctctgtcctctgaaatgtatgtaaagagctataccatccactacagt
gagcagggagtggaatggaaaccatacaggctgaaatcctccatggtg
gacaagatttttgaaggaaatactaataccaaaggacatgtgaagaac
tttttcaaccccccaatcatttccaggtttatccgtgtcattcctaaa
acatggaatcaaagtattgcacttcgcctggaactctttggctgtgat
atttactag.

In some embodiments, the invention provides a recombinant nucleic acid molecule encoding the hFV protein with deletion of amino acids 811-1491 comprising the nucleotide sequence:

(SEQ ID NO: 51)
atgttcccaggctgcccacgcctctgggtcctggtggtcttgggcacc
agctgggtaggctgggggagccaagggacagaagcggcacagctaagg
cagttctacgtggctgctcagggcatcagttggagctaccgacctgag
cccacaaactcaagtttgaatctttctgtaacttcctttaagaaaatt
gtctacagagagtatgaaccatattttaagaaagaaaaaccacaatct
accatttcaggacttcttgggcctactttatatgctgaagtcggagac
atcataaaagttcactttaaaaataaggcagataagcccttgagcatc
catcctcaaggaattaggtacagtaaattatcagaaggtgcttcttac
cttgaccacacattccctgcggagaagatggacgacgctgtggctcca
ggccgagaatacacctatgaatggagtatcagtgaggacagtggaccc
acccatgatgaccctccatgcctcacacacatctattactcccatgaa
aatctgatcgaggatttcaactcggggctgattgggcccctgcttatc
tgtaaaaaagggaccctaactgagggtgggacacagaagacgtttgac
aagcaaatcgtgctactatttgctgtgtttgatgaaagcaagagctgg
agccagtcatcatccctaatgtacacagtcaatggatatgtgaatggg
acaatgccagatataacagtttgtgcccatgaccacatcagctggcat
ctgctgggaatgagctcggggccagaattattctccattcatttcaac
ggccaggtcctggagcagaaccatcataaggtctcagccatcaccctt
gtcagtgctacatccactaccgcaaatatgactgtgggcccagaggga
aagtggatcatatcttctctcaccccaaaacatttgcaagctgggatg
caggcttacattgacattaaaaactgcccaaagaaaaccaggaatctt
aagaaaataactcgtgagcagaggcggcacatgaagaggtgggaatac
ttcattgctgcagaggaagtcatttgggactatgcacctgtaatacca
gcgaatatggacaaaaaatacaggtctcagcatttggataatttctca
aaccaaattggaaaacattataagaaagttatgtacacacagtacgaa
gatgagtccttcaccaaacatacagtgaatcccaatatgaaagaagat
gggattttgggtcctattatcagagcccaggtcagagacacactcaaa
atcgtgttcaaaaatatggccagccgcccctatagcatttaccctcat
ggagtgaccttctcgccttatgaagatgaagtcaactcttctttcacc
tcaggcaggaacaacaccatgatcagagcagttcaaccaggggaaacc
tatacttataagtggaacatcttagagtttgatgaacccacagaaaat
gatgcccagtgcttaacaagaccatactacagtgacgtggacatcatg
agagacatcgcctctgggctaataggactacttctaatctgtaagagc
agatccctggacaggcgaggaatacagagggcagcagacatcgaacag
caggctgtgtttgctgtgtttgatgagaacaaaagctggtaccttgag
gacaacatcaacaagttttgtgaaaatcctgatgaggtgaaacgtgat
gaccccaagttttatgaatcaaacatcatgagcactatcaatggctat
gtgcctgagagcataactactcttggattctgctttgatgacactgtc
cagtggcacttctgtagtgtggggacccagaatgaaattttgaccatc
cacttcactgggcactcattcatctatggaaagaggcatgaggacacc
ttgaccctcttccccatgcgtggagaatctgtgacggtcacaatggat
aatgttggaacttggatgttaacttccatgaattctagtccaagaagc
aaaaagctgaggctgaaattcagggatgttaaatgtatcccagatgat
gatgaagactcatatgagatttttgaacctccagaatctacagtcatg
gctacacggaaaatgcatgatcgtttagaacctgaagatgaagagagt
gatgctgactatgattaccagaacagactggctgcagcattaggaatc
aggagcaacaatggaaacagaagaaattattacattgctgctgaagaa
atatcctgggattattcagaatttgtacaaagggaaacagatattgaa
gactctgatgatattccagaagataccacatataagaaagtagttttt
cgaaagtacctcgacagcacttttaccaaacgtgatcctcgaggggag
tatgaagagcatctcggaattcttggtcctattatcagagctgaagtg
gatgatgttatccaagttcgttttaaaaatttagcatccagaccgtat
tctctacatgcccatggactttcctatgaaaaatcatcagagggaaag
acttatgaagatgactctcctgaatggtttaaggaagataatgctgtt
cagccaaatagcagttatacctacgtatggcatgccactgagcgatca
gggccagaaagtcctggctctgcctgtcgggcttgggcctactactca
gctgtgaacccagaaaaagatattcactcaggcttgataggtcccctc
ctaatctgccaaaaaggaatactacataaggacagcaacatgcctatg
gacatgagagaatttgtcttactatttatgacctttgatgaaaagaag
agctggtactatgaaaagaagtcccgaagttcttggagactcacatcc
tcagaaatgaaaaaatcccatgagtttcacgccattaatgggatgatc
tacagcttgcctggcctgaaaatgtatgagcaagagtgggtgaggtta
cacctgctgaacataggcggctcccaagacattcacgtggttcacttt
cacggccagaccttgctggaaaatggcaataaacagcaccagttaggg
gtctggccccttctgcctggttcatttaaaactcttgaaatgaaggca
tcaaaacctggctggtggctcctaaacacagaggttggagaaaaccag
agagcagggatgcaaacgccatttcttatcatggacagagactgtagg
atgccaatgggactaagcactggtatcatatctgattcacagatcaag
gcttcagagtttctgggttactgggagcccagattagcaagattaaac
aatggtggatcttataatgcttggagtgtagaaaaacttgcagcagaa
tttgcctctaaaccttggatccaggtggacatgcaaaaggaagtcata
atcacagggatccagacccaaggtgccaaacactacctgaagtcctgc
tataccacagagttctatgtagcttacagttccaaccagatcaactgg
cagatcttcaaagggaacagcacaaggaatgtgatgtattttaatggc
aattcagatgcctctacaataaaagagaatcagtttgacccacctatt
gtggctagatatattaggatctctccaactcgagcctataacagacct
acccttcgattggaactgcaaggttgtgaggtaaatggatgttccaca
cccctgggtatggaaaatggaaagatagaaaacaagcaaatcacagct
tcttcgtttaagaaatcttggtggggagattactgggaacccttccgt
gcccgtctgaatgcccagggacgtgtgaatgcctggcaagccaaggca
aacaacaataagcagtggctagaaattgatctactcaagatcaagaag
ataacggcaattataacacagggctgcaagtctctgtcctctgaaatg
tatgtaaagagctataccatccactacagtgagcagggagtggaatgg
aaaccatacaggctgaaatcctccatggtggacaagatttttgaagga
aatactaataccaaaggacatgtgaagaactttttcaaccccccaatc
atttccaggtttatccgtgtcattcctaaaacatggaatcaaagtatt
gcacttcgcctggaactctttggctgtgatatttactag.

In some embodiments, the invention provides a recombinant nucleic acid molecule encoding the hFVa-BDD-SQ protein comprising the nucleotide sequence:

(SEQ ID NO: 52)
atgttcccaggctgcccacgcctctgggtcctggtggtcttgggcacc
agctgggtaggctgggggagccaagggacagaagcggcacagctaagg
cagttctacgtggctgctcagggcatcagttggagctaccgacctgag
cccacaaactcaagtttgaatctttctgtaacttcctttaagaaaatt
gtctacagagagtatgaaccatattttaagaaagaaaaaccacaatct
accatttcaggacttcttgggcctactttatatgctgaagtcggagac
atcataaaagttcactttaaaaataaggcagataagcccttgagcatc
catcctcaaggaattaggtacagtaaattatcagaaggtgcttcttac
cttgaccacacattccctgcggagaagatggacgacgctgtggctcca
ggccgagaatacacctatgaatggagtatcagtgaggacagtggaccc
acccatgatgaccctccatgcctcacacacatctattactcccatgaa
aatctgatcgaggatttcaactcggggctgattgggcccctgcttatc
tgtaaaaaagggaccctaactgagggtgggacacagaagacgtttgac
aagcaaatcgtgctactatttgctgtgtttgatgaaagcaagagctgg
agccagtcatcatccctaatgtacacagtcaatggatatgtgaatggg
acaatgccagatataacagtttgtgcccatgaccacatcagctggcat
ctgctgggaatgagctcggggccagaattattctccattcatttcaac
ggccaggtcctggagcagaaccatcataaggtctcagccatcaccctt
gtcagtgctacatccactaccgcaaatatgactgtgggcccagaggga
aagtggatcatatcttctctcaccccaaaacatttgcaagctgggatg
caggcttacattgacattaaaaactgcccaaagaaaaccaggaatctt
aagaaaataactcgtgagcagaggcggcacatgaagaggtgggaatac
ttcattgctgcagaggaagtcatttgggactatgcacctgtaatacca
gcgaatatggacaaaaaatacaggtctcagcatttggataatttctca
aaccaaattggaaaacattataagaaagttatgtacacacagtacgaa
gatgagtccttcaccaaacatacagtgaatcccaatatgaaagaagat
gggattttgggtcctattatcagagcccaggtcagagacacactcaaa
atcgtgttcaaaaatatggccagccgcccctatagcatttaccctcat
ggagtgaccttctcgccttatgaagatgaagtcaactcttctttcacc
tcaggcaggaacaacaccatgatcagagcagttcaaccaggggaaacc
tatacttataagtggaacatcttagagtttgatgaacccacagaaaat
gatgcccagtgcttaacaagaccatactacagtgacgtggacatcatg
agagacatcgcctctgggctaataggactacttctaatctgtaagagc
agatccctggacaggcgaggaatacagagggcagcagacatcgaacag
caggctgtgtttgctgtgtttgatgagaacaaaagctggtaccttgag
gacaacatcaacaagttttgtgaaaatcctgatgaggtgaaacgtgat
gaccccaagttttatgaatcaaacatcatgagcactatcaatggctat
gtgcctgagagcataactactcttggattctgctttgatgacactgtc
cagtggcacttctgtagtgtggggacccagaatgaaattttgaccatc
cacttcactgggcactcattcatctatggaaagaggcatgaggacacc
ttgaccctcttccccatgcgtggagaatctgtgacggtcacaatggat
aatgttggaacttggatgttaacttccatgaattctagtccaagaagc
aaaaagctgaggctgaaattcagggatgttaaatgtatcccagatgat
gatgaagactcatatgagatttttgaacctccagaatctacagtcatg
gctacacggaaaatgcatgatcgtttagaacctgaagatgaagagagt
gatgctgactatgattaccagaacagactggctgcagcattaggaatc
aggtcattccgaaactcatcattgaatcaggaagaagaagagttcaat
cttactgccctagctctggagaatggcactgaattcgtttcttcaaac
acagatataattgttggttcaaattattcttccccaagtaatattagt
aagttcactgtcaataaccttgcagaacctcagaaagccccttctcac
caacaagccaccacagctggttccccactgagacacctcattggcaag
aactcagttctcaattcttccacagcagagcattccagcccatattct
gaagaccctatagaggatacagattacattgagatcattccaaaggaa
gaggtccagagcagtgaagatgactatgctgaaattgattatgtgccc
tatgatgacccctacaaaactgatgttaggacaaacatcaactcctcc
agagatcctgacaacattgcagcatggtacctccgcagcaacaatgga
aacagaagaaattattacattgctgctgaagaaatatcctgggattat
tcagaatttgtacaaagggaaacagatattgaagactctgatgatatt
ccagaagataccacatataagaaagtagtttttcgaaagtacctcgac
agcacttttaccaaacgtgatcctcgaggggagtatgaagagcatctc
ggaattcttggtcctattatcagagctgaagtggatgatgttatccaa
gttcgttttaaaaatttagcatccagaccgtattctctacatgcccat
ggactttcctatgaaaaatcatcagagggaaagacttatgaagatgac
tctcctgaatggtttaaggaagataatgctgttcagccaaatagcagt
tatacctacgtatggcatgccactgagcgatcagggccagaaagtcct
ggctctgcctgtcgggcttgggcctactactcagctgtgaacccagaa
aaagatattcactcaggcttgataggtcccctcctaatctgccaaaaa
ggaatactacataaggacagcaacatgcctatggacatgagagaattt
gtcttactatttatgacctttgatgaaaagaagagctggtactatgaa
aagaagtcccgaagttcttggagactcacatcctcagaaatgaaaaaa
tcccatgagtttcacgccattaatgggatgatctacagcttgcctggc
ctgaaaatgtatgagcaagagtgggtgaggttacacctgctgaacata
ggcggctcccaagacattcacgtggttcactttcacggccagaccttg
ctggaaaatggcaataaacagcaccagttaggggtctggccccttctg
cctggttcatttaaaactcttgaaatgaaggcatcaaaacctggctgg
tggctcctaaacacagaggttggagaaaaccagagagcagggatgcaa
acgccatttcttatcatggacagagactgtaggatgccaatgggacta
agcactggtatcatatctgattcacagatcaaggcttcagagtttctg
ggttactgggagcccagattagcaagattaaacaatggtggatcttat
aatgcttggagtgtagaaaaacttgcagcagaatttgcctctaaacct
tggatccaggtggacatgcaaaaggaagtcataatcacagggatccag
acccaaggtgccaaacactacctgaagtcctgctataccacagagttc
tatgtagcttacagttccaaccagatcaactggcagatcttcaaaggg
aacagcacaaggaatgtgatgtattttaatggcaattcagatgcctct
acaataaaagagaatcagtttgacccacctattgtggctagatatatt
aggatctctccaactcgagcctataacagacctacccttcgattggaa
ctgcaaggttgtgaggtaaatggatgttccacacccctgggtatggaa
aatggaaagatagaaaacaagcaaatcacagcttcttcgtttaagaaa
tcttggtggggagattactgggaacccttccgtgcccgtctgaatgcc
cagggacgtgtgaatgcctggcaagccaaggcaaacaacaataagcag
tggctagaaattgatctactcaagatcaagaagataacggcaattata
acacagggctgcaagtctctgtcctctgaaatgtatgtaaagagctat
accatccactacagtgagcagggagtggaatggaaaccatacaggctg
aaatcctccatggtggacaagatttttgaaggaaatactaataccaaa
ggacatgtgaagaactttttcaaccccccaatcatttccaggtttatc
cgtgtcattcctaaaacatggaatcaaagtattgcacttcgcctggaa
ctctttggctgtgatatttactag.

In some embodiments, the invention provides a recombinant nucleic acid molecule comprising the nucleotide sequence:

(SEQ ID NO: 53)
atgttcccaggctgcccacgcctctgggtcctggtggtcttgggcacc
agctgggtaggctgggggagccaagggacagaagcggcacagctaagg
cagttctacgtggctgctcagggcatcagttggagctaccgacctgag
cccacaaactcaagtttgaatctttctgtaacttcctttaagaaaatt
gtctacagagagtatgaaccatattttaagaaagaaaaaccacaatct
accatttcaggacttcttgggcctactttatatgctgaagtcggagac
atcataaaagttcactttaaaaataaggcagataagcccttgagcatc
catcctcaaggaattaggtacagtaaattatcagaaggtgcttcttac
cttgaccacacattccctgcggagaagatggacgacgctgtggctcca
ggccgagaatacacctatgaatggagtatcagtgaggacagtggaccc
acccatgatgaccctccatgcctcacacacatctattactcccatgaa
aatctgatcgaggatttcaactcggggctgattgggcccctgcttatc
tgtaaaaaagggaccctaactgagggtgggacacagaagacgtttgac
aagcaaatcgtgctactatttgctgtgtttgatgaaagcaagagctgg
agccagtcatcatccctaatgtacacagtcaatggatatgtgaatggg
acaatgccagatataacagtttgtgcccatgaccacatcagctggcat
ctgctgggaatgagctcggggccagaattattctccattcatttcaac
ggccaggtcctggagcagaaccatcataaggtctcagccatcaccctt
gtcagtgctacatccactaccgcaaatatgactgtgggcccagaggga
aagtggatcatatcttctctcaccccaaaacatttgcaagctgggatg
caggcttacattgacattaaaaactgcccaaagaaaaccaggaatctt
aagaaaataactcgtgagcagaggcggcacatgaagaggtgggaatac
ttcattgctgcagaggaagtcatttgggactatgcacctgtaatacca
gcgaatatggacaaaaaatacaggtctcagcatttggataatttctca
aaccaaattggaaaacattataagaaagttatgtacacacagtacgaa
gatgagtccttcaccaaacatacagtgaatcccaatatgaaagaagat
gggattttgggtcctattatcagagcccaggtcagagacacactcaaa
atcgtgttcaaaaatatggccagccgcccctatagcatttaccctcat
ggagtgaccttctcgccttatgaagatgaagtcaactcttctttcacc
tcaggcaggaacaacaccatgatcagagcagttcaaccaggggaaacc
tatacttataagtggaacatcttagagtttgatgaacccacagaaaat
gatgcccagtgcttaacaagaccatactacagtgacgtggacatcatg
agagacatcgcctctgggctaataggactacttctaatctgtaagagc
agatccctggacaggcgaggaatacagagggcagcagacatcgaacag
caggctgtgtttgctgtgtttgatgagaacaaaagctggtaccttgag
gacaacatcaacaagttttgtgaaaatcctgatgaggtgaaacgtgat
gaccccaagttttatgaatcaaacatcatgagcactatcaatggctat
gtgcctgagagcataactactcttggattctgctttgatgacactgtc
cagtggcacttctgtagtgtggggacccagaatgaaattttgaccatc
cacttcactgggcactcattcatctatggaaagaggcatgaggacacc
ttgaccctcttccccatgcgtggagaatctgtgacggtcacaatggat
aatgttggaacttggatgttaacttccatgaattctagtccaagaagc
aaaaagctgaggctgaaattcagggatgttaaatgtatcccagatgat
gatgaagactcatatgagatttttgaacctccagaatctacagtcatg
gctacacggaaaatgcatgatcgtttagaacctgaagatgaagagagt
gatgctgactatgattaccagaacagactggctgcagcattaggaatc
aggtcattccgaaaccctgacaacattgcagcatggtacctccgcagc
aacaatggaaacagaagaaattattacattgctgctgaagaaatatcc
tgggattattcagaatttgtacaaagggaaacagatattgaagactct
gatgatattccagaagataccacatataagaaagtagtttttcgaaag
tacctcgacagcacttttaccaaacgtgatcctcgaggggagtatgaa
gagcatctcggaattcttggtcctattatcagagctgaagtggatgat
gttatccaagttcgttttaaaaatttagcatccagaccgtattctcta
catgcccatggactttcctatgaaaaatcatcagagggaaagacttat
gaagatgactctcctgaatggtttaaggaagataatgctgttcagcca
aatagcagttatacctacgtatggcatgccactgagcgatcagggcca
gaaagtcctggctctgcctgtcgggcttgggcctactactcagctgtg
aacccagaaaaagatattcactcaggcttgataggtcccctcctaatc
tgccaaaaaggaatactacataaggacagcaacatgcctatggacatg
agagaatttgtcttactatttatgacctttgatgaaaagaagagctgg
tactatgaaaagaagtcccgaagttcttggagactcacatcctcagaa
atgaaaaaatcccatgagtttcacgccattaatgggatgatctacagc
ttgcctggcctgaaaatgtatgagcaagagtgggtgaggttacacctg
ctgaacataggcggctcccaagacattcacgtggttcactttcacggc
cagaccttgctggaaaatggcaataaacagcaccagttaggggtctgg
ccccttctgcctggttcatttaaaactcttgaaatgaaggcatcaaaa
cctggctggtggctcctaaacacagaggttggagaaaaccagagagca
gggatgcaaacgccatttcttatcatggacagagactgtaggatgcca
atgggactaagcactggtatcatatctgattcacagatcaaggcttca
gagtttctgggttactgggagcccagattagcaagattaaacaatggt
ggatcttataatgcttggagtgtagaaaaacttgcagcagaatttgcc
tctaaaccttggatccaggtggacatgcaaaaggaagtcataatcaca
gggatccagacccaaggtgccaaacactacctgaagtcctgctatacc
acagagttctatgtagcttacagttccaaccagatcaactggcagatc
ttcaaagggaacagcacaaggaatgtgatgtattttaatggcaattca
gatgcctctacaataaaagagaatcagtttgacccacctattgtggct
agatatattaggatctctccaactcgagcctataacagacctaccctt
cgattggaactgcaaggttgtgaggtaaatggatgttccacacccctg
ggtatggaaaatggaaagatagaaaacaagcaaatcacagcttcttcg
tttaagaaatcttggtggggagattactgggaacccttccgtgcccgt
ctgaatgcccagggacgtgtgaatgcctggcaagccaaggcaaacaac
aataagcagtggctagaaattgatctactcaagatcaagaagataacg
gcaattataacacagggctgcaagtctctgtcctctgaaatgtatgta
aagagctataccatccactacagtgagcagggagtggaatggaaacca
tacaggctgaaatcctccatggtggacaagatttttgaaggaaatact
aataccaaaggacatgtgaagaactttttcaaccccccaatcatttcc
aggtttatccgtgtcattcctaaaacatggaatcaaagtattgcactt
cgcctggaactctttggctgtgatatttactag.

In some embodiments, the invention provides a recombinant nucleic acid molecule comprising the nucleotide sequence:

(SEQ ID NO: 5)
atgtttcctggatgtccaagactgtgggtcctggtcgtgctgggaact
tcatgggtgggatggggctctcagggaaccgaggccgcacagctgcgc
cagttctatgtggccgcccagggcatctcttggagctaccggccagag
cccaccaatagctccctgaacctgtccgtgacatctttcaagaagatc
gtgtacagagagtatgagccatactttaagaaggagaagccacagagc
accatctccggcctgctgggaccaacactgtacgcagaagtgggcgac
atcatcaaggtgcacttcaagaacaaggccgataagcctctgagcatc
cacccacagggcatccgctactctaagctgagcgagggcgcctcctat
ctggaccacacctttccagccgagaagatggacgatgcagtggcacca
ggaagggagtacacatatgagtggtccatctctgaggacagcggacca
acccacgacgatccaccttgcctgacacacatctactattctcacgag
aatctgatcgaggatttcaacagcggcctgatcggccccctgctgatc
tgtaagaagggcaccctgacagagggcggcacccagaagacatttgac
aagcagatcgtgctgctgttcgccgtgtttgatgagagcaagtcctgg
agccagtctagctccctgatgtacaccgtgaatggctatgtgaacggc
accatgccagacatcacagtgtgcgcccacgatcacatctcttggcac
ctgctgggaatgtctagcggaccagagctgttcagcatccactttaat
ggccaggtgctggagcagaaccaccacaaggtgtccgccatcaccctg
gtgtccgccacatctaccacagccaatatgaccgtgggccccgagggc
aagtggatcatctcctctctgacacctaagcacctgcaggccggcatg
caggcctacatcgacatcaagaattgtcctaagaagacccgcaacctg
aagaagatcacacgggagcagcggagacacatgaagagatgggagtat
ttcatcgccgccgaggaagtgatctgggattacgcccctgtgatccca
gccaacatggacaagaagtataggtcccagcacctggataatttctct
aaccagatcggcaagcactacaagaaagtgatgtatacccagtacgag
gacgagagctttaccaagcacacagtgaatcctaacatgaaggaggac
ggcatcctgggcccaatcatcagggcccaggtgcgcgataccctgaag
atcgtgttcaagaatatggcctccaggccctattctatctaccctcac
ggcgtgacattctctccttacgaggatgaggtgaacagctcctttacc
agcggcagaaacaatacaatgatcagggccgtgcagccaggcgagaca
tacacatataagtggaatatcctggagtttgacgagccaaccgagaac
gatgcccagtgcctgacaagaccctactattccgatgtggacatcatg
agggacatcgcctctggcctgatcggcctgctgctgatctgtaagagc
cgctccctggacaggaggggaatccagagggcagcagatatcgagcag
caggccgtgttcgccgtgtttgacgagaataagtcctggtacctggag
gataatatcaacaagttctgcgagaaccccgatgaggtgaagagagac
gatcctaagttttatgagagcaatatcatgtccaccatcaacggctac
gtgccagagagcatcaccacactgggcttctgctttgacgataccgtg
cagtggcacttctgttctgtgggcacacagaacgagatcctgaccatc
cacttcacaggccacagctttatctatggcaagcgccacgaggacacc
ctgacactgtttcccatgcggggcgagagcgtgaccgtgacaatggat
aatgtgggcacctggatgctgacaagcatgaactctagccccaggtcc
aagaagctgcggctgaagttcagagacgtgaagtgtatccctgacgat
gacgaggattcctacgagatctttgagccacccgagtctaccgtgatg
gccacacgcaagatgcacgaccggctggagcccgaggatgaggagtcc
gatgccgactacgattatcagaacagactggccgccgccctgggaatc
aggagaaagaggcgcaagaggagcaacaatggcaatcggagaaactac
tatatcgccgccgaggagatctcttgggactatagcgagttcgtgcag
cgcgagacagacatcgaggattccgatgacatccccgaggataccaca
tacaagaaggtggtgttccggaagtatctggactctacctttacaaag
cgggatcctagaggcgagtacgaggagcacctgggaatcctgggacca
atcatcagagccgaggtggatgacgtgatccaggtgagattcaagaac
ctggcctccaggccttactctctgcacgcccacggcctgtcctatgag
aagtcctctgagggcaagacctacgaggatgactctcctgagtggttt
aaggaggacaatgccgtgcagccaaacagctcctacacctacgtgtgg
cacgcaacagagagatccggaccagagagccctggatccgcctgcagg
gcctgggcctactatagcgccgtgaatcccgagaaggacatccactcc
ggcctgatcggccctctgctgatctgtcagaagggcatcctgcacaag
gacagcaacatgcctatggatatgagagagttcgtgctgctgttcatg
acctttgatgagaagaagtcttggtactatgagaagaagagcaggtct
agctggcgcctgacatcctctgagatgaagaagtcccacgagtttcac
gccatcaatggcatgatctactctctgccaggcctgaagatgtatgag
caggagtgggtgaggctgcacctgctgaacatcggcggcagccaggac
atccacgtggtgcacttccacggccagaccctgctggagaatggcaac
aagcagcaccagctgggcgtgtggccactgctgccaggcagctttaag
accctggagatgaaggcctccaagcccggctggtggctgctgaatacc
gaagtgggagagaaccagagggcaggaatgcagacaccattcctgatc
atggacagggattgcaggatgccaatgggcctgagcaccggaatcatc
tctgacagccagatcaaggcctccgagtttctgggctattgggagccc
cggctggccagactgaacaatggcggcagctacaatgcatggtccgtg
gagaagctggcagcagagttcgccagcaagccttggatccaggtggat
atgcagaaggaagtgatcatcaccggcatccagacacagggcgccaag
cactacctgaagtcctgttataccacagagttttatgtggcctacagc
tccaatcagatcaactggcagatcttcaagggcaatagcacccggaac
gtgatgtactttaatggcaactctgacgccagcacaatcaaggagaac
cagttcgatcctccaatcgtggccaggtatatccgcatcagccctacc
cgggcctacaatagaccaacactgaggctggagctgcagggctgcgag
gtgaacggctgttccacccctctgggcatggagaatggcaagatcgag
aacaagcagatcacagcctctagcttcaagaagtcttggtggggcgac
tactgggagcccttccgggcccggctgaacgcacagggaagggtgaac
gcctggcaggccaaggccaacaataacaagcagtggctggagatcgat
ctgctgaagatcaagaagatcaccgccatcatcacacagggctgcaag
tccctgtcctctgagatgtatgtgaagtcttacaccatccactatagc
gagcagggcgtggagtggaagccctaccggctgaagagctccatggtg
gacaagatcttcgagggcaataccaacacaaagggccacgtgaagaat
ttctttaacccccctatcatcagccggtttatcagagtgatccctaag
acttggaatcagagtattgccctgcgactggaactgtttggctgtgac
atctattga..

In some embodiments, the amino acid sequence of the invention has been optimized to be expressed at a higher concentration relative to amino acid sequences that have not been optimized. In some embodiments, the FVa sequence of the invention has been optimized to be expressed at a higher concentration relative to amino acid sequences that have not been optimized.

In some embodiments, the invention provides a recombinant nucleic acid construct, comprising the nucleic acid molecule of this invention.

In some embodiments, the invention provides a recombinant nucleic acid molecule, comprising an adeno-associated virus (AAV) 5′ inverted terminal repeat (ITR) and the nucleic acid molecule of this invention operably linked to a promoter and an AAV 3′ ITR.

In some embodiments, the invention provides an AAV particle comprising the nucleic acid molecule, the recombinant nucleic acid construct, or the recombinant nucleic acid molecule of this invention.

In some embodiments, the invention provides a composition comprising the nucleic acid molecule and/or the AAV particle of this invention in a pharmaceutically acceptable carrier. In some embodiments, the composition of this invention further comprises an AAV particle comprising a nucleic acid encoding for FVIIa or a variant or derivative thereof.

In some embodiments, the invention provides a method of administering a nucleic acid molecule to a cell, the method comprising contacting the cell with the nucleic acid molecule and/or AAV particle of this invention, or the composition of this invention.

In some embodiments, the invention provides a method of delivering a nucleic acid molecule to a subject, the method comprising administering to the subject the nucleic acid molecule and/or AAV particle and/or the composition of this invention.

In some embodiments, the invention provides a method of treating bleeding and/or a bleeding disorder in a subject in need thereof, comprising administering to the subject the nucleic acid molecular and/or AAV particle and/or the composition of this invention. In some embodiments, the subject is a human. In some embodiments, the bleeding disorder is hemophilia A, hemophilia B, FV deficiency, FXII deficiency, FXI deficiency, or FVII deficiency. In another embodiment, the bleeding is associated with hemophilia with acquired inhibitors. In another embodiment, the bleeding is associated with thrombocytopenia. In another embodiment, the bleeding is associated with von Willebrand's disease. In another embodiment, the bleeding is associated with severe tissue damage. In another embodiment, the bleeding is associated with severe trauma. In another embodiment, the bleeding is associated with surgery. In another embodiment, the bleeding is associated with laparoscopic surgery. In another embodiment, the bleeding is associated with hemorrhagic gastritis. In another embodiment, the bleeding is profuse uterine bleeding. In another embodiment, the bleeding is occurring in organs with a limited possibility for mechanical hemostasis. In another embodiment, the bleeding is occurring in the brain, inner ear region or eyes. In another embodiment, the bleeding is associated with the process of taking biopsies. In another embodiment, the bleeding is associated with anticoagulant therapy. In another embodiment, the bleeding is associated with childbirth.

In some embodiments, the subject has or is suspected of having or is at risk for developing an inhibitor (wherein the inhibitor is an antibody or other immune system component generated from infusion of factor VIII (FVIII) or factor IX (FIX) making the infused FVIII or FIX ineffective). In some embodiments, the AAV particle or composition of this invention is administered systemically in an amount of about 1×10¹¹ particles to about 1×10¹⁵ particles.

In some embodiments, the invention provides a method of treating excessive and/or uncontrollable bleeding in a subject in need thereof, comprising administering to the subject the nucleic acid molecule, protein, and/or AAV particle and/or the composition of this invention. In some embodiments, the subject has a normally functioning blood clotting cascade, i.e., no clotting factor deficiencies or inhibitors against any of the clotting factors), wherein the bleeding is caused by defective platelet function, thrombocytopenia, von Willebrand's disease, or any other irregularity of the coagulation cascade. In some embodiments, the subject has a normally functioning blood clotting cascade, i.e., no clotting factor deficiencies or inhibitors against any of the clotting factors), wherein the bleeding is caused by tissue damage due to surgery, childbirth, or other trauma.

Also provided are methods of treating a bleeding disorder in a subject having the bleeding disorder by administering the FVa protein of this invention to the subject.

The method of treating the bleeding disorder may include a method of administering to the subject a nucleic acid molecule comprising a nucleotide sequence encoding a FVa protein of this invention.

In some embodiments, the invention provides a method of delivering the nucleic acid molecule, protein, and/or AAV particle of this invention to a subject in need thereof, the method comprising administering the nucleic acid molecule, protein, and/or AAV particle of this invention directly to the subject.

In some embodiments, the invention provides a method for establishing a cell line to produce FVa. Such cell lines include but are not be limited to Chinese hamster ovary (CHO) cells, baby hamster kidney (BHK) cells, SK-HEP cells, HepG2 cells, primary human amniocytes, HKB11 cells and PER.C6 cells. Establishing such a cell line can be done by employing methods known in the art. Exemplary methods include but are not limited to, e.g., U.S. Pat. Nos. 4,784,950 and 7,572,619 and U.S. Patent Application No. 2007/0111312.

### Definitions

Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

To illustrate further, if, for example, the specification indicates that a particular amino acid can be selected from A, G, I, L and/or V, this language also indicates that the amino acid can be selected from any subset of these amino acid(s) for example A, G, I or L; A, G, I or V; A or G; only L; etc. as if each such sub combination is expressly set forth herein. Moreover, such language also indicates that one or more of the specified amino acids can be disclaimed (e.g., by negative proviso). For example, in particular embodiments the amino acid is not A, G or I; is not A; is not G or V; etc. as if each such possible disclaimer is expressly set forth herein.

The designation of all amino acid positions in the AAV capsid proteins in the AAV vectors and recombinant AAV nucleic acid molecules of the invention is with respect to VP1 capsid subunit numbering (native AAV2 VP1 capsid protein: GenBank Accession No. AAC03780 or YP680426). It will be understood by those skilled in the art that modifications as described herein if inserted into the AAV cap gene may result in modifications in the VP1, VP2 and/or VP3 capsid subunits. Alternatively, the capsid subunits can be expressed independently to achieve modification in only one or two of the capsid subunits (VP1, VP2, VP3, VP1+VP2, VP1+VP3, or VP2+VP3).

As used herein, “a,” “an” or “the” can mean one or more than one. For example, “a” cell can mean a single cell or a multiplicity of cells.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of dose (e.g., an amount of a non-viral vector) and the like, is meant to encompass variations of ±20%, ±10%, ±5%, ±1%, ±0.5%, or even ±0.1% of the specified amount.

As used herein, the transitional phrase “consisting essentially of” means that the scope of a claim is to be interpreted to encompass the specified materials or steps recited in the claim, “and those that do not materially affect the basic and novel characteristic(s)” of the claimed invention. See, In re Herz, 537 F.2d 549, 551-52, 190 USPQ 461, 463 (CCPA 1976) (emphasis in the original); see also MPEP § 2111.03. Thus, the term “consisting essentially of” when used in a claim of this invention is not intended to be interpreted to be equivalent to “comprising.”

As used herein, the terms “reduce,” “reduces,” “reduction,” “diminish,” “inhibit” and similar terms mean a decrease of at least about 5%, 10%, 15%, 20%, 25%, 35%, 50%, 75%, 80%, 85%, 90%, 95%, 97% or more.

As used herein, the terms “enhance,” “enhances,” “enhancement” and similar terms indicate an increase of at least about 25%, 50%, 75%, 100%, 150%, 200%, 300%, 400%, 500% or more.

The term “parvovirus” as used herein encompasses the family Parvoviridae, including autonomously replicating parvoviruses and dependoviruses. The autonomous parvoviruses include members of the genera Parvovirus, Erythrovirus, Densovirus, Iteravirus, and Contravirus. Exemplary autonomous parvoviruses include, but are not limited to, minute virus of mouse, bovine parvovirus, canine parvovirus, chicken parvovirus, feline panleukopenia virus, feline parvovirus, goose parvovirus, H1 parvovirus, muscovy duck parvovirus, B19 virus, and any other autonomous parvovirus now known or later discovered. Other autonomous parvoviruses are known to those skilled in the art. See, e.g., BERNARD N. FIELDS et al., VIROLOGY, Volume 2, Chapter 69 (4th ed., Lippincott-Raven Publishers).

As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, avian AAV, bovine AAV, canine AAV, equine AAV, ovine AAV, and any other AAV now known or later discovered. See, e.g., BERNARD N. FIELDS et al., VIROLOGY, volume 2, chapter 69 (4th ed., Lippincott-Raven Publishers). A number of additional AAV serotypes and clades have been identified (see, e.g., Gao et al., (2004) J. Virology 78:6381-6388; Moris et al., (2004) Virology 33-:375-383; and Table 3).

The genomic sequences of various serotypes of AAV and the autonomous parvoviruses, as well as the sequences of the native terminal repeats (TRs), Rep proteins, and capsid subunits are known in the art. Such sequences may be found in the literature or in public databases such as GenBank. See, e.g., GenBank Accession Numbers NC_002077, NC_001401, NC_001729, NC_001863, NC_001829, NC_001862, NC_000883, NC_001701, NC_001510, NC_006152, NC_006261, AF063497, U89790, AF043303, AF028705, AF028704, J02275, J01901, J02275, X01457, AF288061, AH009962, AY028226, AY028223, NC_001358, NC_001540, AF513851, AF513852, AY530579; the disclosures of which are incorporated by reference herein for teaching parvovirus and AAV nucleic acid and amino acid sequences. See also, e.g., Srivistava et al. (1983) J. Virology 45:555; Chiorini et al. (1998) J. Virology 71:6823; Chiorini et al. (1999) J. Virology 73:1309; Bantel-Schaal et al. (1999) J. Virology 73:939; Xiao et al. (1999) J. Virology 73:3994; Muramatsu et al. (1996) Virology 221:208; Shade et al. (1986) J. Virol. 58:921; Gao et al. (2002) Proc. Nat. Acad. Sci. USA 99:11854; Moris et al. (2004) Virology 33:375-383; international patent publications WO 00/28061, WO 99/61601, WO 98/11244; and U.S. Pat. No. 6,156,303; the disclosures of which are incorporated by reference herein for teaching parvovirus and AAV nucleic acid and amino acid sequences. See also Table 1.

The capsid structures of autonomous parvoviruses and AAV are described in more detail in BERNARD N. FIELDS et al. VIROLOGY, volume 2, chapters 69 & 70 (4th ed., Lippincott-Raven Publishers). See also, description of the crystal structure of AAV2 (Xie et al. (2002) Proc. Nat. Acad. Sci. 99:10405-10); AAV4 (Padron et al. (2005) J. Virol. 79: 5047-58); AAV5 (Walters et al. (2004) J. Virol. 78:3361-71); and CPV (Xie et al. (1996) J. Mol. Biol. 6:497-520 and Tsao et al. (1991) Science 251:1456-64).

The term “tropism” as used herein refers to preferential entry of the virus into certain cells or tissues, optionally followed by expression (e.g., transcription and, optionally, translation) of a sequence(s) carried by the viral genome in the cell, e.g., for a recombinant virus, expression of a heterologous nucleic acid(s) of interest.

As used herein, the term “polypeptide” encompasses both peptides and proteins, unless indicated otherwise.

A “polynucleotide” is a sequence of nucleotide bases, and may be RNA, DNA or DNA-RNA hybrid sequences (including both naturally occurring and non-naturally occurring nucleotides), but in representative embodiments are either single or double stranded DNA sequences.

As used herein, an “isolated” polynucleotide (e.g., an “isolated DNA” or an “isolated RNA”) means a polynucleotide at least partially separated from at least some of the other components of the naturally occurring organism or virus, for example, the cell or viral structural components or other polypeptides or nucleic acids commonly found associated with the polynucleotide. In representative embodiments an “isolated” nucleotide is enriched by at least about 10-fold, 100-fold, 1000-fold, 10,000-fold or more as compared with the starting material.

Likewise, an “isolated” polypeptide means a polypeptide that is at least partially separated from at least some of the other components of the naturally occurring organism or virus, for example, the cell or viral structural components or other polypeptides or nucleic acids commonly found associated with the polypeptide. In representative embodiments an “isolated” polypeptide is enriched by at least about 10-fold, 100-fold, 1000-fold, 10,000-fold or more as compared with the starting material.

An “isolated cell” refers to a cell that is separated from other components with which it is normally associated in its natural state. For example, an isolated cell can be a cell in culture medium and/or a cell in a pharmaceutically acceptable carrier of this invention. Thus, an isolated cell can be delivered to and/or introduced into a subject. In some embodiments, an isolated cell can be a cell that is removed from a subject and manipulated as described herein ex vivo and then returned to the subject.

As used herein, by “isolate” or “purify” (or grammatical equivalents) a virus vector or virus particle or population of virus particles, it is meant that the virus vector or virus particle or population of virus particles is at least partially separated from at least some of the other components in the starting material. In representative embodiments an “isolated” or “purified” virus vector or virus particle or population of virus particles is enriched by at least about 10-fold, 100-fold, 1000-fold, 10,000-fold or more as compared with the starting material.

A “therapeutic polypeptide” is a polypeptide that can alleviate, reduce, prevent, delay and/or stabilize symptoms that result from an absence or defect in a protein in a cell or subject and/or is a polypeptide that otherwise confers a benefit to a subject, e.g., anti-cancer effects or improvement in transplant survivability or induction of an immune response.

By the terms “treat,” “treating” or “treatment of” (and grammatical variations thereof) it is meant that the severity of the subject's condition is reduced, at least partially improved or stabilized and/or that some alleviation, mitigation, decrease or stabilization in at least one clinical symptom is achieved and/or there is a delay in the progression of the disease or disorder.

The terms “prevent,” “preventing” and “prevention” (and grammatical variations thereof) refer to prevention and/or delay of the onset of a disease, disorder and/or a clinical symptom(s) in a subject and/or a reduction in the severity of the onset of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset are substantially less than what would occur in the absence of the present invention.

A “treatment effective” or “effective” amount as used herein is an amount that is sufficient to provide some improvement or benefit to the subject. Alternatively stated, a “treatment effective” or “effective” amount is an amount that will provide some alleviation, mitigation, decrease or stabilization in at least one clinical symptom in the subject. Those skilled in the art will appreciate that the therapeutic effects need not be complete or curative, as long as some benefit is provided to the subject.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some preventative benefit is provided to the subject.

The term “bleeding episode” is meant to include uncontrolled and excessive bleeding. Bleeding episodes may be a major problem both in connection with surgery and other forms of tissue damage. Uncontrolled and excessive bleeding may occur in subjects having a normal coagulation system and subjects having coagulation or bleeding disorders.

As used herein the term “bleeding disorder” reflects any defect, congenital, acquired or induced, of cellular, physiological, or molecular origin that is manifested in bleedings. Examples are clotting factor deficiencies (e.g., hemophilia A and B or deficiency of coagulation Factors XI or VII), clotting factor inhibitors, defective platelet function, thrombocytopenia, von Willebrand's disease, or bleeding induced by surgery or trauma.

As used therein the term “excessive bleedings” refers to bleeding that occurs in subjects with a normally functioning blood clotting cascade (no clotting factor deficiencies or inhibitors against any of the coagulation factors) and may be caused by a defective platelet function, thrombocytopenia or von Willebrand's disease. In such cases, the bleedings may be likened to those bleedings caused by hemophilia because the haemostatic system, as in hemophilia, lacks or has abnormal essential clotting “compounds” (such as platelets or von Willebrand factor protein), causing major bleedings. In subjects who experience extensive tissue damage in association with surgery or trauma, the normal haemostatic mechanism may be overwhelmed by the demand of immediate hemostasis and they may develop bleeding in spite of a normal haemostatic mechanism. Achieving satisfactory hemostasis also is a problem when bleedings occur in organs such as the brain, inner ear region and eyes, with limited possibility for surgical hemostasis. The same problem may arise in the process of taking biopsies from various organs (liver, lung, tumor tissue, gastrointestinal tract) as well as in laparoscopic surgery. Common for all these situations is the difficulty to provide hemostasis by surgical techniques (sutures, clips, etc.), which also is the case when bleeding is diffuse (hemorrhagic gastritis and profuse uterine bleeding). Acute and profuse bleedings may also occur in subjects on anticoagulant therapy in whom a defective hemostasis has been induced by the therapy given. Such subjects may need surgical interventions in case the anticoagulant effect has to be counteracted rapidly. Radical retropubic prostatectomy is a commonly performed procedure for subjects with localized prostate cancer. The operation is frequently complicated by significant and sometimes massive blood loss. The considerable blood loss during prostatectomy is mainly related to the complicated anatomical situation, with various densely vascularized sites that are not easily accessible for surgical hemostasis, and which may result in diffuse bleeding from a large area. Also, intracerebral hemorrhage is the least treatable form of stroke and is associated with high mortality and hematoma growth in the first few hours following intracerebral hemorrhage. Another situation that may cause problems in the case of unsatisfactory hemostasis is when subjects with a normal haemostatic mechanism are given anticoagulant therapy to prevent thromboembolic disease. Such therapy may include heparin, other forms of proteoglycans, warfarin or other forms of vitamin K-antagonists as well as aspirin and other platelet aggregation inhibitors.

The terms “nucleotide sequence of interest (NOI),” “heterologous nucleotide sequence” and “heterologous nucleic acid molecule” are used interchangeably herein and refer to a nucleic acid sequence that is not naturally occurring (e.g., engineered). Generally, the NOI, heterologous nucleic acid molecule or heterologous nucleotide sequence comprises an open reading frame that encodes a polypeptide and/or nontranslated RNA of interest (e.g., for delivery to a cell and/or subject).

As used herein, the terms “virus vector,” “vector” or “gene delivery vector” refer to a virus (e.g., AAV) particle that functions as a nucleic acid delivery vehicle, and which comprises a viral genome (e.g., viral DNA [vDNA]) and/or replicon nucleic acid molecule packaged within a virus particle. Alternatively, in some contexts, the term “vector” may be used to refer to the vector genome/vDNA alone.

The term “vector,” as used herein, means any nucleic acid entity capable of amplification in a host cell. Thus, the vector may be an autonomously replicating vector, i.e., a vector, which exists as an extrachromosomal entity, the replication of which is independent of chromosomal replication, e.g., a plasmid. Alternatively, the vector may be one which, when introduced into a host cell, is integrated into the host cell genome and replicated together with the chromosome(s) into which it has been integrated. The choice of vector will often depend on the host cell into which it is to be introduced. Vectors include, but are not limited to plasmid vectors, phage vectors, viruses or cosmid vectors. Vectors usually contain a replication origin and at least one selectable gene, i.e., a gene which encodes a product which is readily detectable or the presence of which is essential for cell growth

A “rAAV vector genome” or “rAAV genome” is an AAV genome (i.e., vDNA) that comprises at least one terminal repeat (e.g., two terminal repeats) and one or more heterologous nucleotide sequences. rAAV vectors generally require only the 145 base terminal repeat(s) (TR(s)) in cis to generate virus. All other viral sequences are dispensable and may be supplied in trans (Muzyczka, (1992) Curr. Topics Microbiol. Immunol. 158:97). Typically, the rAAV vector genome will only retain the minimal TR sequence(s) so as to maximize the size of the transgene that can be efficiently packaged by the vector. The structural and non-structural protein coding sequences may be provided in trans (e.g., from a vector, such as a plasmid, or by stably integrating the sequences into a packaging cell). The rAAV vector genome optionally comprises two AAV TRs, which generally will be at the 5′ and 3′ ends of the heterologous nucleotide sequence(s), but need not be contiguous thereto. The TRs can be the same or different from each other.

A “rAAV particle” comprises a rAAV vector genome packaged within an AAV capsid.

The term “terminal repeat” or “TR” or “inverted terminal repeat (ITR)” includes any viral terminal repeat or synthetic sequence that forms a hairpin structure and functions as an inverted terminal repeat (i.e., mediates the desired functions such as replication, virus packaging, integration and/or provirus rescue, and the like). The TR can be an AAV TR or a non-AAV TR. For example, a non-AAV TR sequence such as those of other parvoviruses (e.g., canine parvovirus (CPV), mouse parvovirus (MVM), human parvovirus B-19) or any other suitable virus sequence (e.g., the SV40 hairpin that serves as the origin of SV40 replication) can be used as a TR, which can further be modified by truncation, substitution, deletion, insertion and/or addition. Further, the TR can be partially or completely synthetic, such as the “double-D sequence” as described in U.S. Pat. No. 5,478,745 to Samulski et al., which is hereby incorporated by reference in its entirety.

An “AAV terminal repeat” or “AAV TR” may be from any AAV, including but not limited to serotypes 1, 2, 3, 4, 5, 6, 7, 8, 9, 10, 11 or 12 or any other AAV now known or later discovered (see, e.g., Table 3). An AAV terminal repeat need not have the native terminal repeat sequence (e.g., a native AAV TR sequence may be altered by insertion, deletion, truncation and/or missense mutations), as long as the terminal repeat mediates the desired functions, e.g., replication, virus packaging, integration, and/or provirus rescue, and the like.

AAV proteins VP1, VP2 and VP3 are capsid proteins that interact together to form an AAV capsid of an icosahedral symmetry. VP1.5 is an AAV capsid protein described in US Publication No. 2014/0037585, which is hereby incorporated by reference in its entirety

The virus vectors of the invention can further be “targeted” virus vectors (e.g., having a directed tropism) and/or a “hybrid” parvovirus (i.e., in which the viral TRs and viral capsid are from different parvoviruses) as described in international patent publication WO 00/28004 and Chao et al., (2000) Molecular Therapy 2:619, which is hereby incorporated by reference in its entirety.

The virus vectors of the invention can further be duplexed parvovirus particles as described in international patent publication WO 01/92551 (the disclosure of which is incorporated herein by reference in its entirety). Thus, in some embodiments, double stranded (duplex) genomes can be packaged into the virus capsids of the invention.

Further, the viral capsid or genomic elements can contain other modifications, including insertions, deletions and/or substitutions.

A “chimeric’ capsid protein as used herein means an AAV capsid protein that has been modified by substitutions in one or more (e.g., 2, 3, 4, 5, 6, 7, 8, 9, 10, etc.) amino acid residues in the amino acid sequence of the capsid protein relative to wild type, as well as insertions and/or deletions of one or more (e.g., 2, 3, 4, 5, 6, 7, 8, 9, 10, etc.) amino acid residues in the amino acid sequence relative to wild type. In some embodiments, complete or partial domains, functional regions, epitopes, etc., from one AAV serotype can replace the corresponding wild type domain, functional region, epitope, etc. of a different AAV serotype, in any combination, to produce a chimeric capsid protein of this invention. Production of a chimeric capsid protein can be carried out according to protocols well known in the art and a large number of chimeric capsid proteins are described in the literature as well as herein that can be included in the capsid of this invention.

As used herein, the term “amino acid” or “amino acid residue” encompasses any naturally occurring amino acid, modified forms thereof, and synthetic amino acids.

Naturally occurring, levorotatory (L-) amino acids are shown in Table 2.

Alternatively, the amino acid can be a modified amino acid residue (nonlimiting examples are shown in Table 4) and/or can be an amino acid that is modified by post-translation modification (e.g., acetylation, amidation, formylation, hydroxylation, methylation, phosphorylation or sulfatation).

Further, the non-naturally occurring amino acid can be an “unnatural” amino acid as described by Wang et al., Annu Rev Biophys Biomol Struct. 35:225-49 (2006)). These unnatural amino acids can advantageously be used to chemically link molecules of interest to the AAV capsid protein.

In some embodiments, the AAV vector of this invention can be a synthetic viral vector designed to display a range of desirable phenotypes that are suitable for different in vitro and in vivo applications. Thus, in one embodiment, the present invention provides an AAV particle comprising an adeno-associated virus (AAV) capsid, wherein the capsid comprises capsid protein VP1, wherein said capsid protein VP1 is from one or more than one first AAV serotype and capsid protein VP3, wherein said capsid protein VP3 is from one or more than one second AAV serotype and wherein at least one of said first AAV serotype is different from at least one of said second AAV serotype, in any combination.

In some embodiments, the AAV particle can comprise a capsid that comprises capsid protein VP2, wherein said capsid protein VP2 is from one or more than one third AAV serotype, wherein at least one of said one or more than one third AAV serotype is different from said first AAV serotype and/or said second AAV serotype, in any combination. In some embodiments, the AAV capsid described herein can comprise capsid protein VP1.5. VP1.5 is described in US Patent Publication No. 20140037585 and the amino acid sequence of VP1.5 is provided herein.

In some embodiments, the AAV particle of this invention can comprise a capsid that comprises capsid protein VP1.5, wherein said capsid protein VP1.5 is from one or more than one fourth AAV serotype, wherein at least one of said one or more than one fourth AAV serotype is different from said first AAV serotype and/or said second AAV serotype, in any combination. In some embodiments, the AAV capsid protein described herein can comprise capsid protein VP2.

The present invention also provides an AAV vector of this invention, comprising an AAV capsid wherein the capsid comprises capsid protein VP1, wherein said capsid protein VP1 is from one or more than one first AAV serotype and capsid protein VP2, wherein said capsid protein VP2 is from one or more than one second AAV serotype and wherein at least one of said first AAV serotype is different from at least one of said second AAV serotype, in any combination.

In some embodiments, the AAV vector of this invention can comprise a capsid that comprises capsid protein VP3, wherein said capsid protein VP3 is from one or more than one third AAV serotype, wherein at least one of said one or more than one third AAV serotype is different from said first AAV serotype and/or said second AAV serotype, in any combination. In some embodiments, the AAV capsid described herein can comprise capsid protein VP 1.5.

The present invention further provides an AAV vector that comprises an adeno-associated virus (AAV) capsid, wherein the capsid comprises capsid protein VP1, wherein said capsid protein VP1 is from one or more than one first AAV serotype and capsid protein VP1.5, wherein said capsid protein VP1.5 is from one or more than one second AAV serotype and wherein at least one of said first AAV serotype is different from at least one of said second AAV serotype, in any combination.

In some embodiments, the AAV vector of this invention can comprise a capsid that comprises capsid protein VP3, wherein said capsid protein VP3 is from one or more than one third AAV serotype, wherein at least one of said one or more than one third AAV serotype is different from said first AAV serotype and/or said second AAV serotype, in any combination. In some embodiments, the AAV capsid protein described herein can comprise capsid protein VP2.

In some embodiments of the capsid of the AAV vector described herein, said one or more than one first AAV serotype, said one or more than one second AAV serotype, said one or more than one third AAV serotype and said one or more than one fourth AAV serotype are selected from the group consisting of the AAV serotypes listed in Table 1, in any combination.

In some embodiments of the AAV vector of this invention, the AAV capsid described herein lacks capsid protein VP2.

In some embodiments of the AAV vector of this invention, the capsid can comprise a chimeric capsid VP1 protein, a chimeric capsid VP2 protein, a chimeric capsid VP3 protein and/or a chimeric capsid VP1.5 protein.

The present invention further provides a composition, which can be a pharmaceutical formulation comprising the virus vector or AAV particle of this invention and a pharmaceutically acceptable carrier.

Heterologous molecules (e.g., nucleic acid, proteins, peptides, etc.) are defined as those that are not naturally found in an AAV infection, e.g., those not encoded by a wild-type AAV genome. Further, therapeutically useful molecules can be associated with a transgene for transfer of the molecules into host target cells. Such associated molecules can include DNA and/or RNA.

The modified capsid proteins and capsids can further comprise any other modification, now known or later identified. Those skilled in the art will appreciate that for some AAV capsid proteins the corresponding modification will be an insertion and/or a substitution, depending on whether the corresponding amino acid positions are partially or completely present in the virus or, alternatively, are completely absent. Likewise, when modifying AAV other than AAV2, the specific amino acid position(s) may be different than the position in AAV2 (see, e.g., Table 3). As discussed elsewhere herein, the corresponding amino acid position(s) will be readily apparent to those skilled in the art using well-known techniques. Nonlimiting examples of corresponding positions in a number of other AAV serotypes are shown in Table 3 (Position 2).

In representative embodiments, the virus vector of this invention is a recombinant virus vector comprising a heterologous nucleic acid encoding a polypeptide of this invention, such as a FVa protein. Recombinant virus vectors are described in more detail below.

It will be understood by those skilled in the art that, in certain embodiments, the capsid proteins, virus capsids, virus vectors and virus particles of the invention exclude those capsid proteins, capsids, virus vectors and virus particles as they would be present or found in their native state.

### Methods of Producing Virus Vectors.

Viral vectors have been used in a wide variety of gene delivery applications in cells, as well as living animal subjects. Viral vectors that can be used include, but are not limited to, retrovirus, lentivirus (e.g., lentivirus 5′ long terminal repeats (LTR), adeno-associated virus (AAV), poxvirus, alphavirus, baculovirus, vaccinia virus, herpes virus, Epstein-Barr virus, and adenovirus vectors (e.g., adenovirus 5′ ITR). Non-viral vectors include plasmids, liposomes, electrically charged lipids (cytofectins), nucleic acid-protein complexes, and biopolymers. In addition to a nucleic acid of interest, a vector may also comprise one or more regulatory regions, and/or selectable markers useful in selecting, measuring, and monitoring nucleic acid transfer results (delivery to specific tissues, duration of expression, etc.).

Vectors may be introduced into the desired cells by methods known in the art, e.g., transfection, electroporation, microinjection, transduction, cell fusion, DEAE dextran, calcium phosphate precipitation, lipofection (lysosome fusion), use of a gene gun, or a nucleic acid vector transporter (see, e.g., Wu et al., J. Biol. Chem. 267:963 (1992); Wu et al., J. Biol. Chem. 263:14621 (1988); and Hartmut et al., Canadian Patent Application No. 2,012,311, filed Mar. 15, 1990). These methods can be employed singly or in any combination and/or order.

In various embodiments, other molecules can be used for facilitating delivery of a nucleic acid in vivo, such as a cationic oligopeptide (e.g., WO95/21931), peptides derived from nucleic acid binding proteins (e.g., WO96/25508), and/or a cationic polymer (e.g., WO95/21931).

It is also possible to introduce a vector in vivo as naked nucleic acid (see U.S. Pat. Nos. 5,693,622, 5,589,466 and 5,580,859; incorporated by reference herein). Receptor-mediated nucleic acid delivery approaches can also be used (Curiel et al., Hum. Gene Ther. 3:147 (1992); Wu et al., J Biol. Chem. 262:4429 (1987)).

In some embodiments, the present invention provides methods of producing virus particles and vectors of this invention. In particular, the present invention provides a method of making an AAV particle, comprising: a) transfecting a host cell with one or more plasmids that provide, in combination all functions and genes needed to assemble AAV particles; b) introducing one or more nucleic acid constructs into a packaging cell line or producer cell line to provide, in combination, all functions and genes needed to assemble AAV particles; c) introducing into a host cell one or more recombinant baculovirus vectors that provide in combination all functions and genes needed to assemble AAV particles; and/or d) introducing into a host cell one or more recombinant herpesvirus vectors that provide in combination all functions and genes needed to assemble AAV particles. Nonlimiting examples of various methods of making the virus vectors of this invention are described in Clement and Greiger (“Manufacturing of recombinant adeno-associated viral vectors for clinical trials” Mol. Ther. Methods Clin Dev. 3:16002 (2016)) and in Greiger et al. (“Production of recombinant adeno-associated virus vectors using suspension HEK293 cells and continuous harvest of vector from the culture media for GMP FIX and FLT1 clinical vector” Mol Ther 24(2):287-297 (2016)), the entire contents of which are incorporated by reference herein.

In one representative embodiment, the present invention provides a method of producing an AAV particle, the method comprising providing to a cell: (a) a nucleic acid template comprising at least one TR sequence (e.g., AAV TR sequence), and (b) AAV sequences sufficient for replication of the nucleic acid template and encapsidation into AAV capsids (e.g., AAV rep sequences and AAV cap sequences encoding the AAV capsids of the invention). Optionally, the nucleic acid template further comprises at least one heterologous nucleic acid sequence. In particular embodiments, the nucleic acid template comprises two AAV ITR sequences, which are located 5′ and 3′ to the heterologous nucleic acid sequence (if present), although they need not be directly contiguous thereto.

The nucleic acid template and AAV rep and cap sequences are provided under conditions such that virus vector comprising the nucleic acid template packaged within the AAV capsid is produced in the cell. The method can further comprise the step of collecting the virus vector from the cell. The virus vector can be collected from the medium and/or by lysing the cells.

The cell can be a cell that is permissive for AAV viral replication. Any suitable cell known in the art may be employed. In particular embodiments, the cell is a mammalian cell. As another option, the cell can be a trans-complementing packaging cell line that provides functions deleted from a replication-defective helper virus, e.g., 293 cells or other Ela trans-complementing cells.

The AAV replication and capsid sequences may be provided by any method known in the art. Current protocols typically express the AAV rep/cap genes on a single plasmid. The AAV replication and packaging sequences need not be provided together, although it may be convenient to do so. The AAV rep and/or cap sequences may be provided by any viral or non-viral vector. For example, the rep/cap sequences may be provided by a hybrid adenovirus or herpesvirus vector (e.g., inserted into the E1a or E3 regions of a deleted adenovirus vector). Epstein Barr virus (EBV) vectors may also be employed to express the AAV cap and rep genes. One advantage of this method is that EBV vectors are episomal, yet will maintain a high copy number throughout successive cell divisions (i.e., are stably integrated into the cell as extra-chromosomal elements, designated as an “EBV based nuclear episome,” see Margolski, (1992) Curr. Top. Microbiol. Immun. 158:67). As a further alternative, the rep/cap sequences may be stably incorporated into a cell.

Typically the AAV rep/cap sequences will not be flanked by the TRs, to prevent rescue and/or packaging of these sequences.

The nucleic acid template can be provided to the cell using any method known in the art. For example, as mentioned above the template can be supplied by a non-viral (e.g., plasmid) or viral vector. In particular embodiments, the nucleic acid template is supplied by a herpesvirus or adenovirus vector (e.g., inserted into the Ela or E3 regions of a deleted adenovirus). As another illustration, Palombo et al. (1998) J. Virology 72:5025, describes a baculovirus vector carrying a reporter gene flanked by the AAV TRs. EBV vectors may also be employed to deliver the template, as described above with respect to the rep/cap genes.

In another representative embodiment, the nucleic acid template is provided by a replicating rAAV virus. In still other embodiments, an AAV provirus comprising the nucleic acid template is stably integrated into the chromosome of the cell.

To enhance virus titers, helper virus functions (e.g., adenovirus or herpesvirus) that promote a productive AAV infection can be provided to the cell. Helper virus sequences necessary for AAV replication are known in the art. Typically, these sequences will be provided by a helper adenovirus or herpesvirus vector. Alternatively, the adenovirus or herpesvirus sequences can be provided by another non-viral or viral vector, e.g., as a non-infectious adenovirus miniplasmid that carries all of the helper genes that promote efficient AAV production as described by Ferrari et al. (1997) Nature Med. 3:1295, and U.S. Pat. Nos. 6,040,183 and 6,093,570.

Further, the helper virus functions may be provided by a packaging cell with the helper sequences embedded in the chromosome or maintained as a stable extrachromosomal element. In some embodiments, the helper virus sequences cannot be packaged into AAV virions, e.g., are not flanked by TRs.

Those skilled in the art will appreciate that it may be advantageous to provide the AAV replication and capsid sequences and the helper virus sequences (e.g., adenovirus sequences) on a single helper construct. This helper construct may be a non-viral or viral construct. As one nonlimiting illustration, the helper construct can be a hybrid adenovirus or hybrid herpesvirus comprising the AAV rep/cap genes.

In one embodiment, the AAV rep/cap sequences and the adenovirus helper sequences are supplied by a single adenovirus helper vector. This vector can further comprise the nucleic acid template. The AAV rep/cap sequences and/or the rAAV template can be inserted into a deleted region (e.g., the Ela or E3 regions) of the adenovirus.

In a further embodiment, the AAV rep/cap sequences and the adenovirus helper sequences are supplied by a single adenovirus helper vector. According to this embodiment, the rAAV template can be provided as a plasmid template.

In another illustrative embodiment, the AAV rep/cap sequences and adenovirus helper sequences are provided by a single adenovirus helper vector, and the rAAV template is integrated into the cell as a provirus. Alternatively, the rAAV template is provided by an EBV vector that is maintained within the cell as an extrachromosomal element (e.g., as an EBV based nuclear episome).

In a further exemplary embodiment, the AAV rep/cap sequences and adenovirus helper sequences are provided by a single adenovirus helper. The rAAV template can be provided as a separate replicating viral vector. For example, the rAAV template can be provided by a rAAV particle or a second recombinant adenovirus particle.

According to the foregoing methods, the hybrid adenovirus vector typically comprises the adenovirus 5′ and 3′ cis sequences sufficient for adenovirus replication and packaging (i.e., the adenovirus terminal repeats and PAC sequence). The AAV rep/cap sequences and if present the rAAV template are embedded in the adenovirus backbone and are flanked by the 5′ and 3′ cis sequences, so that these sequences may be packaged into adenovirus capsids. As described above, the adenovirus helper sequences and the AAV rep/cap sequences are generally not flanked by TRs so that these sequences are not packaged into the AAV virions.

Herpesvirus may also be used as a helper virus in AAV packaging methods. Hybrid herpesviruses encoding the AAV Rep protein(s) may advantageously facilitate scalable AAV vector production schemes. A hybrid herpes simplex virus type I (HSV-1) vector expressing the AAV-2 rep and cap genes has been described (Conway et al. (1999) Gene Therapy 6:986 and WO 00/17377.

As a further alternative, the virus vectors of the invention can be produced in insect cells using baculovirus vectors to deliver the rep/cap genes and rAAV template as described, for example, by Urabe et al. (2002) Human Gene Therapy 13:1935-43.

Viral vector stocks free of contaminating helper virus may be obtained by any method known in the art. For example, AAV and helper virus may be readily differentiated based on size. AAV may also be separated away from helper virus based on affinity for a heparin substrate (Zolotukhin et al. (1999) Gene Therapy 6:973). Deleted replication-defective helper viruses can be used so that any contaminating helper virus is not replication competent. As a further alternative, an adenovirus helper lacking late gene expression may be employed, as only adenovirus early gene expression is required to mediate packaging of AAV virus. Adenovirus mutants defective for late gene expression are known in the art (e.g., ts100K and ts149 adenovirus mutants).

### Recombinant Virus Vectors.

The virus vectors of the present invention are useful for the delivery of nucleic acid molecules to cells in vitro, ex vivo, and in vivo. In particular, the virus vectors can be advantageously employed to deliver or transfer nucleic acid molecules to animal cells, including mammalian cells.

Non-limiting examples of heterologous nucleic acid sequence(s) of interest of this invention include clotting factors (e.g., Factor V, Factor VII, Factor VIII, Factor IX, Factor X, Factor IX, Factor X, etc.), which may be delivered in the virus vectors of the present invention. Nucleic acid molecules of interest include nucleic acid molecules encoding polypeptides, including therapeutic (e.g., for medical or veterinary uses) and/or immunogenic (e.g., for vaccines) polypeptides.

In some embodiments, viral vectors of this invention can also be used to deliver monoclonal antibodies and antibody fragments, for example, an antibody or antibody fragment directed against one or more constituents and/or components present in the coagulation/clotting cascade.

The virus vector may also comprise a heterologous nucleic acid molecule that shares homology with and recombines with a locus on a host cell chromosome. This approach can be utilized, for example, to correct a genetic defect in the host cell.

The present invention also provides virus vectors that express an immunogenic polypeptide, peptide and/or epitope, e.g., for vaccination. The nucleic acid molecule may encode any immunogen of interest known in the art that is related to a bleeding disorder.

The use of parvoviruses as vaccine vectors is known in the art (see, e.g., Miyamura et al., (1994) Proc. Nat. Acad. Sci USA 91:8507; U.S. Pat. No. 5,916,563 to Young et al., U.S. Pat. No. 5,905,040 to Mazzara et al., U.S. Pat. Nos. 5,882,652, 5,863,541 to Samulski et al.). The antigen may be presented in the parvovirus capsid. Alternatively, the immunogen or antigen may be expressed from a heterologous nucleic acid molecule introduced into a recombinant vector genome. Any immunogen or antigen of interest as described herein and/or as is known in the art can be provided by the virus vector of the present invention. An immunogenic polypeptide can be any polypeptide, peptide, and/or epitope suitable for eliciting an immune response and/or protecting the subject from a bleeding disorder.

As a further alternative, the heterologous nucleic acid molecule can encode any polypeptide, peptide and/or epitope that is desirably produced in a cell in vitro, ex vivo, or in vivo. For example, the virus vectors may be introduced into cultured cells and the expressed gene product isolated therefrom.

It will be understood by those skilled in the art that the heterologous nucleic acid molecule(s) of interest can be operably associated with appropriate control sequences. For example, the heterologous nucleic acid molecule can be operably associated with expression control elements, such as transcription/translation control signals, origins of replication, polyadenylation signals, internal ribosome entry sites (IRES), signal peptides, promoters, and/or enhancers, and the like.

Further, regulated expression of the heterologous nucleic acid molecule(s) of interest can be achieved at the post-transcriptional level, e.g., by regulating selective splicing of different introns by the presence or absence of an oligonucleotide, small molecule and/or other compound that selectively blocks splicing activity at specific sites (e.g., as described in WO 2006/119137).

Those skilled in the art will appreciate that a variety of promoter/enhancer elements can be used depending on the level and tissue-specific expression desired. The promoter/enhancer can be constitutive or inducible, depending on the pattern of expression desired. The promoter/enhancer can be native or foreign and can be a natural or a synthetic sequence. By foreign, it is intended that the transcriptional initiation region is not found in the wild-type host into which the transcriptional initiation region is introduced.

In particular embodiments, the promoter/enhancer elements can be native to the target cell or subject to be treated. In representative embodiments, the promoters/enhancer element can be native to the heterologous nucleic acid sequence. The promoter/enhancer element is generally chosen so that it functions in the target cell(s) of interest. Further, in particular embodiments the promoter/enhancer element is a mammalian promoter/enhancer element. The promoter/enhancer element may be constitutive or inducible.

Inducible expression control elements are typically advantageous in those applications in which it is desirable to provide regulation over expression of the heterologous nucleic acid sequence(s). Inducible promoters/enhancer elements for gene delivery can be tissue-specific or -preferred promoter/enhancer elements. Other inducible promoter/enhancer elements include hormone-inducible and metal-inducible elements. Exemplary inducible promoters/enhancer elements include, but are not limited to, a Tet on/off element, a RU486-inducible promoter, an ecdysone-inducible promoter, a rapamycin-inducible promoter, and a metallothionein promoter.

Examples of promoters include, but are not limited to sequences selected from TTR (transthyretin); TTR/mvm (TTR promoter with Minute Virus of Mice (MVM) intron); HLP (Human liver specific promoter, A 251-bp fragment containing a 34-bp core enhancer from the human apolipoprotein hepatic control region and a modified 217-bp α-1-antitrypsin (AIAT) promoter); Ch19-AIAT (122 bp from AAV integrated site from chromosome 19 and 185 bp of AIAT promoter); pHU1-1 (a minimal human 243 bp cellular small nuclear RNA promoter); the human elongation factor 1alpha promoter; herpes simplex thymidine kinase (Tk) promoter (pDLZ2); Tk promoter linked to Enhancer I of hepatitis B virus; a synthetic, basic albumin promoter; a synthetically derived short liver-specific promoter/enhancer of 368 bp from the insulin-like growth factor-binding protein followed by a 175-bp chimeric intron (IGBP/enh/intron); beta-actin minimum promoter; a cytomegalovirus promoter (CMV); a human β-actin promoter with a CMV enhancer (CB); liver-specific human alpha1 anti-trypsin promoter (HAAT) and the liver-specific hepatic control region (HCR) enhancer/human alpha1 anti-trypsin promoter complex (HCRHAAT); human insulin-like growth factor binding protein (IGFBP) promoter; HCR-hAAT (the human apolipoprotein E/C-I gene locus control region (HCR) and the human α1 antitrypsin promoter (hAAT) with a chicken β actin/rabbit β globin composite intron); U1a (small nuclear RNA promoter); Histone H2 promote; U1b2 small nuclear RNA promoter; Histone H3 promoter; α-Antitrypsin promoter; Human factor IX promoter with liver transcription factor-responsive oligomers; CM1 promoter (HCR/ApoE enhancer/α-antitrypsin promoter); LSP (liver specific promoter: TH-binding globulin promoter/α1-microglobulin/bikunin enhancer); or any ubiquitous promoters that drive protein expression in the liver and muscles as well as in any cell lines.

In embodiments wherein the heterologous nucleic acid sequence(s) is transcribed and then translated in the target cells, specific initiation signals are generally included for efficient translation of inserted protein coding sequences. These exogenous translational control sequences, which may include the ATG initiation codon and adjacent sequences, can be of a variety of origins, both natural and synthetic.

Examples of signal peptides include, but are not limited to, signal peptides comprising an amino acid sequence selected from hFV: MFPGCPRLWVLVVLGTSWVGWGSQGTEA (SEQ ID NO:1); hFVII: MVSQALRLLCLLLGLQGCLA (SEQ ID NO:6); hFIX: MQRVNMIMAESPGLITICLLGYLLSAEC (SEQ ID NO:7); MQIELSTCFFLCLLRFCFS (SEQ ID NO:8); Human fibrinogen-alpha chain: MFSMRIVCLVLSVVGTAWT (SEQ ID NO:9); Human fibrinogen-beta chain: MKRMVSWSFHKLKTMKHLLLLLLCVFLVKS (SEQ ID NO:10); Human fibrinogen-gamma chain: MSWSLHPRNLILYFYALLFLSSTCVA (SEQ ID NO:11); hFXII: MRALLLLGFLLVSLESTLS (SEQ ID NO:12); Protein C: MWQLTSLLLFVATWGISG (SEQ ID NO:13); Protein S: MRVLGGRCGALLACLLLVLPVSEA (SEQ ID NO:14); Thrombin: MAHVRGLQLPGCLALAALCSLVHS (SEQ ID NO:15); Anti-thrombin: MYSNVIGTVTSGKRKVYLLSLLLIGFWDCVTC (SEQ ID NO:16); Serum albumin: MKWVTFISLLFLFSSAYS (SEQ ID NO:17); Transferrin: MRLAVGALLVCAVLGLCLA (SEQ ID NO:18); Alpha-1 antitrypsin: MPSSVSWGILLLAGLCCLVPVSLA (SEQ ID NO:19); Fibronectin: MLRGPGPGLLLLAVQCLGTAVPSTGASKSKR (SEQ ID NO:20); Alpha-1-microglobulin: MRSLGALLLLLSACLAVSA (SEQ ID NO:21); Alpha 1-antichymotrypsin: MERMLPLLALGLLAAGFCPAVLC (SEQ ID NO:22); Apo A: MKAAVLTLAVLFLTGSQA (SEQ ID NO:23); Apo B: MDPPRPALLALLALPALLLLLLAGARA (SEQ ID NO:24); Apo E: MKVLWAALLVTFLAGCQA (SEQ ID NO:25); Alpha-fetoprotein: MKWVESIFLIFLLNFTES (SEQ ID NO:26); C-reactive protein: MEKLLCFLVLTSLSHAFG (SEQ ID NO:27); Plasminogen: MEHKEVVLLLLLFLKSGQG (SEQ ID NO:28); Ceruloplasmin: MKILILGIFLFLCSTPAWA (SEQ ID NO:29); Complement C1q subunit A: MEGPRGWLVLCVLAISLASMVT (SEQ ID NO:30); Complement C2: MGPLMVLFCLLFLYPGLADS (SEQ ID NO:31); Complement C3: MGPTSGPSLLLLLLTHLPLALG (SEQ ID NO:32); Complement C4A: MRLLWGLIWASSFFTLSLQ (SEQ ID NO:33); Complement C5: MGLLGILCFLIFLGKTWG (SEQ ID NO:34); Complement C6: MARRSVLYFILLNALINKGQA (SEQ ID NO:35); Complement C7: MKVISLFILVGFIGEFQSFSSA (SEQ ID NO:36); Complement C8A: MFAVVFFILSLMTCQPGVTA (SEQ ID NO:37); Complement C9: MSACRSFAVAICILEISILTA (SEQ ID NO:38); α2-antiplasmin: MALLWGLLVLSWSCLQGPCSVFSPVSA (SEQ ID NO:39); Transcortin: MPLLLYTCLLWLPTSGLWTVQA (SEQ ID NO:40); Haptoglobin: MSALGAVIALLLWGQLFA (SEQ ID NO:41); Hemopexin: MARVLGAPVALGLWSLCWSLAIA (SEQ ID NO:42); IGF binding protein 1: MSEVPVARVWLVLLLLTVQVGVTAG (IGFBP2-7) (SEQ ID NO:43); Transthyretin: MASHRLLLLCLAGLVFVSEA (SEQ ID NO:44); Insulin-like growth factor 1 (IGF-1): MGKISSLPTQLFKCCFCDFLK (SEQ ID NO:45); Thrombopoietin: MELTELLLVVMLLLTARLTLS (SEQ ID NO:46); β2 microglobulin: MSRSVALAVLALLSLSGLEA (SEQ ID NO:47); alpha-2-Macroglobulin: MGKNKLLHPSLVLLLLVLLPTDA (SEQ ID NO:48); and any signal peptides from any other serum protein.

The virus vectors according to the present invention provide a means for delivering heterologous nucleic acid molecules into a broad range of cells, including dividing and non-dividing cells. The virus vectors can be employed to deliver a nucleic acid molecule of interest to a cell in vitro, e.g., to produce a polypeptide in vitro or for ex vivo or in vivo gene therapy. The virus vectors are additionally useful in a method of delivering a nucleic acid to a subject in need thereof, e.g., to express an immunogenic or therapeutic polypeptide or a functional RNA. In this manner, the polypeptide or functional RNA can be produced in vivo in the subject. The subject can be in need of the polypeptide because the subject has a deficiency of the polypeptide. Further, the method can be practiced because the production of the polypeptide or functional RNA in the subject may impart some beneficial effect.

The virus vectors can also be used to produce a polypeptide of interest or functional RNA in cultured cells or in a subject (e.g., using the subject as a bioreactor to produce the polypeptide or to observe the effects of the functional RNA on the subject, for example, in connection with screening methods).

In general, the virus vectors of the present invention can be employed to deliver a heterologous nucleic acid molecule encoding a polypeptide or functional RNA to treat and/or prevent any bleeding disorder or disease state for which it is beneficial to deliver a therapeutic polypeptide or functional RNA. Illustrative disease states include, but are not limited to: hemophilia A (Factor VIII), hemophilia B (Factor IX), FV deficiency, FXII deficiency, FXI deficiency, and FVII deficiency.

In some embodiments, the virus vectors of the present invention can be employed to deliver a heterologous nucleic acid molecule encoding a polypeptide or functional RNA to treat and/or prevent a bleeding disorder or disease state for which it is beneficial to deliver a therapeutic polypeptide or functional RNA. In some embodiments, the heterologous nucleic acid molecule encodes activated clotting factor VII (FVIIa). In some embodiments, the heterologous nucleic acid molecule encodes activated clotting factor V (FVa). In some embodiments, a combination of virus vectors comprising different heterologous nucleic acid molecules encoding for different polypeptides is delivered to treat and/or present a bleeding disorder or disease. For example, in some embodiments, a combination of virus vectors comprising heterologous nucleic acid molecules encoding FVIIa and FVa are delivered as a single construct or multiple constructs to treat a bleeding disorder or disease.

In some embodiments, only a portion of the full-length cDNA of a clotting factor is delivered when viral vectors are employed as a delivery tool. In some embodiments wherenever the viral vector is an AAV vector, due to the size limitation of the AAV virion package (i.e., less than 4.7 kb) certain domains may have to be deleted. For example, deletion of the B-domain in the human FV cDNA is facilitates delivery of FVa by an AAV vector. Thus, in some embodiments, the nucleic acid molecule comprises a synthetic protein molecule wherein a heavy chain (HC) domain of FVa (e.g., SEQ ID NO: 2) is linked via a linker sequence to a light chain (LC) domain of VFa (e.g., SEQ ID NO: 3). The linker sequence can vary. For example, in some embodiments, the linker sequence can comprise a furin recognition motif (e.g., amino acid sequence RKRRKR) (SEQ ID NO: 49)). In some embodiments, the linker sequence can comprise a 2A self-cleavage peptide from foot-and-mouth disease virus, or equine rhinitis A virus, or porcine teschovirus, or hosea asigna virus.

In some embodiments, the linker sequence can comprise (GGGS)ₙ and/or (GS)ₙ subunits in any combination and n can be 1 or any number greater than 1 (e.g., 2, 3, 4, 5, 6, 7, 8, 9, 10, 11, 12, 13, 14, 15, 16, 17, 18, 19, 20, 25, 30, 35, 40, 45, 50, etc). In some embodiments, the linker sequence can comprise any length of snake B domain; any length of human FV B domain N-terminus within 100 aa; any length of human FV B domain C-terminus within about 100 aa; any length of human FVIII B domain N-terminus within about 100 aa; any length of human FVIII B domain C-terminus within about 100 aa; and any combinations thereof.

Gene transfer has substantial potential use for understanding and providing therapy for disease states. In general, inherited diseases, such as hemophilia A and B, in which defective genes are known and have been cloned typically fall into two classes: deficiency states, usually of enzymes, which are generally inherited in a recessive manner, and unbalanced states, which may involve regulatory or structural proteins, and which are typically inherited in a dominant manner. For deficiency state diseases, gene transfer can be used to bring a normal gene into affected tissues for replacement therapy, as well as to create animal models for the disease using antisense mutations. For unbalanced disease states, gene transfer can be used to create a disease state in a model system, which can then be used in efforts to counteract the disease state. Thus, virus vectors according to the present invention permit the treatment and/or prevention of genetic diseases, such as Hemophilia A and B.

The virus vectors of the present invention can also be used for various non-therapeutic purposes, including but not limited to use in protocols to assess gene targeting, clearance, transcription, translation, etc., as would be apparent to one skilled in the art. The virus vectors can also be used for the purpose of evaluating safety (spread, toxicity, immunogenicity, etc.). Such data, for example, are considered by the United States Food and Drug Administration as part of the regulatory approval process prior to evaluation of clinical efficacy.

As a further aspect, the virus vectors of the present invention may be used to produce an immune response in a subject. According to this embodiment, a virus vector comprising a heterologous nucleic acid sequence encoding an immunogenic polypeptide can be administered to a subject, and an active immune response is mounted by the subject against the immunogenic polypeptide Immunogenic polypeptides are as described hereinabove. In some embodiments, a protective immune response is elicited.

An “active immune response” or “active immunity” is characterized by “participation of host tissues and cells after an encounter with the immunogen. It involves differentiation and proliferation of immunocompetent cells in lymphoreticular tissues, which lead to synthesis of antibody or the development of cell-mediated reactivity, or both.” Herbert B. Herscowitz, Immunophysiology: Cell Function and Cellular Interactions in Antibody Formation, in IMMUNOLOGY: BASIC PROCESSES 117 (Joseph A. Bellanti ed., 1985). Alternatively stated, an active immune response is mounted by the host after exposure to an immunogen by infection or by vaccination. Active immunity can be contrasted with passive immunity, which is acquired through the “transfer of preformed substances (antibody, transfer factor, thymic graft, interleukin-2) from an actively immunized host to a non-immune host.” Id.

A “protective” immune response or “protective” immunity as used herein indicates that the immune response confers some benefit to the subject in that it prevents or reduces the incidence of disease. Alternatively, a protective immune response or protective immunity may be useful in the treatment and/or prevention of bleeding disorders that are acquired (e.g., autoimmune disease) rather than genetic, e.g., acute hemophilia. The protective effects may be complete or partial, as long as the benefits of the treatment outweigh any disadvantages thereof. In some embodiments, the virus vector or cell comprising the heterologous nucleic acid molecule can be administered in an immunogenically effective amount.

### Pharmaceutical Formulations and Administration.

The clotting factor Va protein according to the present invention may be used to control bleeding disorders which have several causes such as clotting factor deficiencies (e.g., hemophilia A and B or deficiency of coagulation factors XI or VII) or clotting factor inhibitors, or they may be used to control excessive bleeding occurring in subjects with a normally functioning blood clotting cascade (no clotting factor deficiencies or inhibitors against any of the coagulation factors). The bleedings may be caused by a defective platelet function, thrombocytopenia or von Willebrand's disease. They may also be seen in subjects in whom an increased fibrinolytic activity has been induced by various stimuli.

In subjects who experience extensive tissue damage in association with surgery, childbirth, or trauma, the haemostatic mechanism may be overwhelmed by the demand of immediate hemostasis and they may develop bleedings in spite of a normal haemostatic mechanism. Achieving satisfactory hemostasis is also a problem when bleedings occur in organs such as the brain, inner ear region and eyes and may also be a problem in cases of diffuse bleedings (hemorrhagic gastritis and profuse uterine bleeding) when it is difficult to identify the source. The same problem may arise in the process of taking biopsies from various organs (liver, lung, tumor tissue, gastrointestinal tract) as well as in laparoscopic surgery. These situations share the difficulty of providing hemostasis by surgical techniques (sutures, clips, etc.). Acute and profuse bleedings may also occur in subjects on anticoagulant therapy in whom a defective hemostasis has been induced by the therapy given. Such subjects may need surgical interventions in case the anticoagulant effect has to be counteracted rapidly. Another situation that may cause problems in the case of unsatisfactory hemostasis is when subjects with a normal haemostatic mechanism are given anticoagulant therapy to prevent thromboembolic disease. Such therapy may include heparin, other forms of proteoglycans, warfarin or other forms of vitamin K-antagonists as well as aspirin and other platelet aggregation inhibitors.

The present invention provides a method of administering a nucleic acid molecule to a cell, the method comprising contacting the cell with the virus vector, the AAV particle, the composition and/or the pharmaceutical formulation of this invention.

The present invention further provides a method of delivering a nucleic acid to a subject, the method comprising administering to the subject the virus vector, the AAV particle, the composition and/or the pharmaceutical formulation of this invention.

Delivery of the vector into a subject may be either direct, in which case the patient is directly exposed to the vector or a delivery complex, or indirect, in which case, cells are first transformed with the vector in vitro, and then transplanted into the patient. These two approaches are known, respectively, as in vivo and ex vivo gene therapy.

In one embodiment, the vector is directly administered in vivo, where it enters the cells of the subject and mediates expression of the gene. This can be accomplished by any of numerous methods known in the art and discussed above, e.g., by constructing it as part of an appropriate expression vector and administering it so that it becomes intracellular, e.g., by infection using a defective or attenuated retroviral or other viral vector (see, U.S. Pat. No. 4,980,286), or by direct injection of naked DNA, or by use of microparticle bombardment (e.g., a gene gun; Biolistic, Dupont); or coating with lipids or cell-surface receptors or transfecting agents, encapsulation in biopolymers (e.g., poly-β-1-64-N-acetylglucosamine polysaccharide; see U.S. Pat. No. 5,635,493), encapsulation in liposomes, microparticles, or microcapsules; by administering it in linkage to a peptide or other ligand known to enter the nucleus; or by administering it in linkage to a ligand subject to receptor-mediated endocytosis (Wu and Wu, J. Biol. Chem. (1987) 62:4429-4432), etc. In another embodiment, a nucleic acid-ligand complex can be formed in which the ligand comprises a fusogenic viral peptide to disrupt endosomes, allowing the nucleic acid to avoid lysosomal degradation, or cationic 12-mer peptides, e.g., derived from antennapedia, that can be used to transfer therapeutic DNA into cells (Mi et al., Mol. Therapy 2000, 2:339-47). In yet another embodiment, the nucleic acid can be targeted in vivo for cell specific uptake and expression, by targeting a specific receptor (see, e.g., PCT Publication Nos. WO 92/06180, WO 92/22635, WO 92/20316 and WO 93/14188). Additionally, a technique referred to as magnetofection may be used to deliver vectors to mammals. This technique associates the vectors with superparamagnetic nanoparticles for delivery under the influence of magnetic fields. This application reduces the delivery time and enhances vector efficacy (Scherer et al. Gene Therapy (2002) 9:102-9).

In one embodiment, the nucleic acid can be administered using a lipid carrier. Lipid carriers can be associated with naked nucleic acids (e.g., plasmid DNA) to facilitate passage through cellular membranes. Cationic, anionic, or neutral lipids can be used for this purpose. However, cationic lipids are suitable because they have been shown to associate better with DNA which, generally, has a negative charge. Cationic lipids have also been shown to mediate intracellular delivery of plasmid DNA (Feigner and Ringold, Nature 1989; 337:387). Intravenous injection of cationic lipid-plasmid complexes into mice has been shown to result in expression of the DNA in lung (Brigham et al. Am. J. Med. Sci. (1989) 298:278). See also, Osaka et al. J. Pharm. Sci. (1996) 85(6):612-618; San et al. Human Gene Therapy (1993) 4:781-788; Senior et al. Biochemica et Biophysica Acta (1991) 1070:173-179); Kabanov and Kabanov. Bioconjugate Chem. (1995) 6:7-20; Liu et al. Pharmaceut. Res. (1996) 13; Remy et al. Bioconjugate Chem. (1994) 5:647-654; Behr. Bioconjugate Chem (1994) 5:382-389; Wyman et al. Biochem. (1997) 36:3008-3017; U.S. Pat. Nos. 5,939,401; 6,331,524.

Representative cationic lipids include those disclosed, for example, in U.S. Pat. Nos. 5,283,185; and 5,767,099, the entire disclosures of which are incorporated herein by reference. In one embodiment, the cationic lipid is N₄-spermine cholesteryl carbamate (GL-67) disclosed in U.S. Pat. No. 5,767,099. Additional suitable lipids include N₄-spermidine cholestryl carbamate (GL-53) and 1-(N₄-spermine)-2,3-dilaurylglycerol carbamate (GL-89).

In some embodiments, the present invention further provides a method of directly delivering one or more clotting factor proteins to a subject, the method comprising administering to the subject the one or more clotting factor proteins. In some embodiments, the clotting factor being delivered is FVa alone or in combination with FVIIa.

The subject of this invention can be any animal and in some embodiments, the subject is a mammal and in some embodiments, the subject is a human. In some embodiments, the subject has or is at risk for a disorder that can be treated by gene therapy protocols. Nonlimiting examples of such disorders include hemophilia A and hemophilia B, as well as other hemophiliac and bleeding disorders.

In representative embodiments, the subject is “in need of” the methods of the invention. For example, in some embodiments, the subject is in need of a clotting factor. In some embodiments, the subject has to a bleeding disorder and/or disease and optionally has developed inhibitors for certain clotting factors (e.g., FVIII inhibitors)

In particular embodiments, the present invention provides a pharmaceutical composition comprising a virus vector and/or capsid and/or AAV particle and/or protein of the invention in a pharmaceutically acceptable carrier and, optionally, other medicinal agents, pharmaceutical agents, stabilizing agents, buffers, carriers, adjuvants, diluents, etc. For injection, the carrier will typically be a liquid. For other methods of administration, the carrier may be either solid or liquid. For inhalation administration, the carrier will be respirable, and optionally can be in solid or liquid particulate form. For administration to a subject or for other pharmaceutical uses, the carrier will be sterile and/or physiologically compatible.

By “pharmaceutically acceptable” it is meant a material that is not toxic or otherwise undesirable, i.e., the material may be administered to a subject without causing any undesirable biological effects.

One aspect of the present invention is a method of introducing a nucleic acid molecule into a cell in vitro. The virus vector may be introduced into the cells at the appropriate multiplicity of infection according to standard transduction methods suitable for the particular target cells. Titers of virus vector to administer can vary, depending upon the target cell type and number, and the particular virus vector, and can be determined by those of skill in the art without undue experimentation. In representative embodiments, at least about 10³ infectious units, optionally at least about 10⁵ infectious units are introduced to the cell.

The cell(s) into which the virus vector is introduced can be of any type, including but not limited to neural cells (including cells of the peripheral and central nervous systems, in particular, brain cells such as neurons and oligodendricytes), lung cells, cells of the eye (including retinal cells, retinal pigment epithelium, and corneal cells), epithelial cells (e.g., gut and respiratory epithelial cells), muscle cells (e.g., skeletal muscle cells, cardiac muscle cells, smooth muscle cells and/or diaphragm muscle cells), dendritic cells, pancreatic cells (including islet cells), hepatic cells, myocardial cells, bone cells (e.g., bone marrow stem cells), hematopoietic stem cells, spleen cells, keratinocytes, fibroblasts, endothelial cells, prostate cells, germ cells, and the like. In representative embodiments, the cell can be any progenitor cell. As a further possibility, the cell can be a stem cell (e.g., neural stem cell, liver stem cell). As still a further alternative, the cell can be a cancer or tumor cell. Moreover, the cell can be from any species of origin, as indicated above.

The virus vector can be introduced into cells in vitro for the purpose of administering the modified cell to a subject. In particular embodiments, the cells have been removed from a subject, the virus vector is introduced therein, and the cells are then administered back into the subject. Methods of removing cells from subject for manipulation ex vivo, followed by introduction back into the subject are known in the art (see, e.g., U.S. Pat. No. 5,399,346). Alternatively, the recombinant virus vector can be introduced into cells from a donor subject, into cultured cells, or into cells from any other suitable source, and the cells are administered to a subject in need thereof (i.e., a “recipient” subject).

Suitable cells for ex vivo nucleic acid delivery are as described above. Dosages of the cells to administer to a subject will vary upon the age, condition and species of the subject, the type of cell, the nucleic acid being expressed by the cell, the mode of administration, and the like. Typically, at least about 10² to about 10⁸ cells or at least about 10³ to about 10⁶ cells will be administered per dose in a pharmaceutically acceptable carrier. In particular embodiments, the cells transduced with the virus vector are administered to the subject in a treatment effective or prevention effective amount in combination with a pharmaceutical carrier.

In some embodiments, the virus vector is introduced into a cell and the cell can be administered to a subject to elicit an immunogenic response against the delivered polypeptide (e.g., expressed as a transgene or in the capsid). Typically, a quantity of cells expressing an immunogenically effective amount of the polypeptide in combination with a pharmaceutically acceptable carrier is administered. An “immunogenically effective amount” is an amount of the expressed polypeptide that is sufficient to evoke an active immune response against the polypeptide in the subject to which the pharmaceutical formulation is administered. In particular embodiments, the dosage is sufficient to produce a protective immune response (as defined above). The degree of protection conferred need not be complete or permanent, as long as the benefits of administering the immunogenic polypeptide outweigh any disadvantages thereof.

A further aspect of the invention is a method of administering the virus vector and/or virus capsid to subjects. Administration of the virus vectors and/or capsids according to the present invention to a human subject or an animal in need thereof can be by any means known in the art. Optionally, the virus vector and/or capsid are delivered in a treatment effective or prevention effective dose in a pharmaceutically acceptable carrier.

The virus vectors and/or capsids of the invention can further be administered to elicit an immunogenic response (e.g., as a vaccine). Typically, immunogenic compositions of the present invention comprise an immunogenically effective amount of virus vector and/or capsid in combination with a pharmaceutically acceptable carrier. Optionally, the dosage is sufficient to produce a protective immune response (as defined above). The degree of protection conferred need not be complete or permanent, as long as the benefits of administering the immunogenic polypeptide outweigh any disadvantages thereof. Subjects and immunogens are as described above.

Dosages of the virus vector and/or capsid to be administered to a subject depend upon the mode of administration, the disease or condition to be treated and/or prevented, the individual subject's condition, the particular virus vector or capsid, and the nucleic acid to be delivered, and the like, and can be determined in a routine manner. Exemplary doses for achieving therapeutic effects are titers of at least about 10⁵, 10⁶, 10⁷, 10⁸, 10⁹, 10¹⁰, 10¹¹, 10¹², 10³, 10¹⁴, 10¹⁵ transducing units, optionally about 10¹¹ to about 10¹⁵ transducing units.

In particular embodiments, more than one administration (e.g., two, three, four, five, six, seven, eight, nine, 10, etc., or more administrations) may be employed to achieve the desired level of gene expression over a period of various intervals, e.g., hourly, daily, weekly, monthly, yearly, etc.

Exemplary modes of administration include oral, rectal, transmucosal, intranasal, inhalation (e.g., via an aerosol), buccal (e.g., sublingual), vaginal, intrathecal, intraocular, transdermal, in utero (or in ovo), parenteral (e.g., intravenous, subcutaneous, intradermal, intramuscular (i.e., including administration to skeletal, diaphragm and/or cardiac muscle), intradermal, intrapleural, intracerebral, and intraarticular, topical (e.g., to both skin and mucosal surfaces, including airway surfaces, and transdermal administration), intralymphatic, and the like, as well as direct tissue or organ injection (e.g., to liver, skeletal muscle, cardiac muscle, diaphragm muscle or brain). In some embodiments, the pharmaceutical composition and/or protein is directly administered into the joint (e.g., intraarticular). The most suitable route in any given case will depend on the nature and severity of the condition being treated and/or prevented and on the nature of the particular vector that is being used.

The virus vector and/or capsid can be delivered by intravenous administration, intra-arterial administration, intraperitoneal administration, limb perfusion, (optionally, isolated limb perfusion of a leg and/or arm; see, e.g., Arruda et al. (2005) Blood 105:3458-3464), and/or direct intramuscular injection. In particular embodiments, the virus vector and/or capsid is administered to a limb (arm and/or leg) of a subject (e.g., a subject with muscular dystrophy such as DMD) by limb perfusion, optionally isolated limb perfusion (e.g., by intravenous or intra-articular administration). In embodiments of the invention, the virus vectors and/or capsids of the invention can advantageously be administered without employing “hydrodynamic” techniques. Tissue delivery (e.g., to muscle) of prior art vectors is often enhanced by hydrodynamic techniques (e.g., intravenous/intravenous administration in a large volume), which increase pressure in the vasculature and facilitate the ability of the vector to cross the endothelial cell barrier. In particular embodiments, the viral vectors and/or capsids of the invention can be administered in the absence of hydrodynamic techniques such as high volume infusions and/or elevated intravascular pressure (e.g., greater than normal systolic pressure, for example, less than or equal to a 5%, 10%, 15%, 20%, 25% increase in intravascular pressure over normal systolic pressure). Such methods may reduce or avoid the side effects associated with hydrodynamic techniques such as edema, nerve damage and/or compartment syndrome.

Injectables can be prepared in conventional forms, either as liquid solutions or suspensions, solid forms suitable for solution or suspension in liquid prior to injection, or as emulsions. Alternatively, one may administer the virus vector and/or virus capsids of the invention in a local rather than systemic manner, for example, in a depot or sustained-release formulation. Further, the virus vector and/or virus capsid can be delivered adhered to a surgically implantable matrix (e.g., as described in U.S. Patent Publication No. US-2004-0013645-A1).

The present subject matter will be now be described more fully hereinafter with reference to the accompanying EXAMPLES, in which representative embodiments of the presently disclosed subject matter are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

### EXAMPLES

The following EXAMPLES provide illustrative embodiments. Certain aspects of the following EXAMPLES are disclosed in terms of techniques and procedures found or contemplated by the present inventors to work well in the practice of the embodiments. In light of the present disclosure and the general level of skill in the art, those of skill will appreciate that the following EXAMPLES are intended to be exemplary only and that numerous changes, modifications, and alterations can be employed without departing from the scope of the presently claimed subject matter.

### Example 1: Optimization of AAV/FVa Cassettes for Phenotypic Correction in Hemophilic Mice with Inhibitors

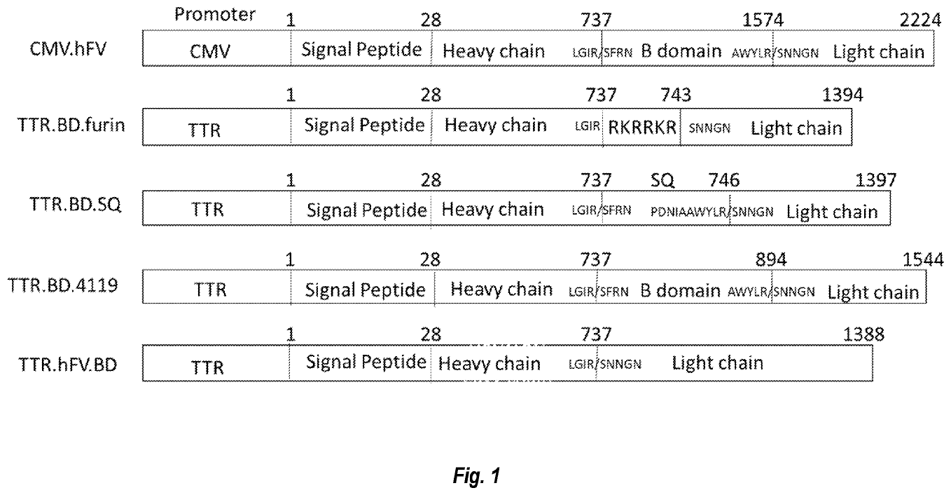

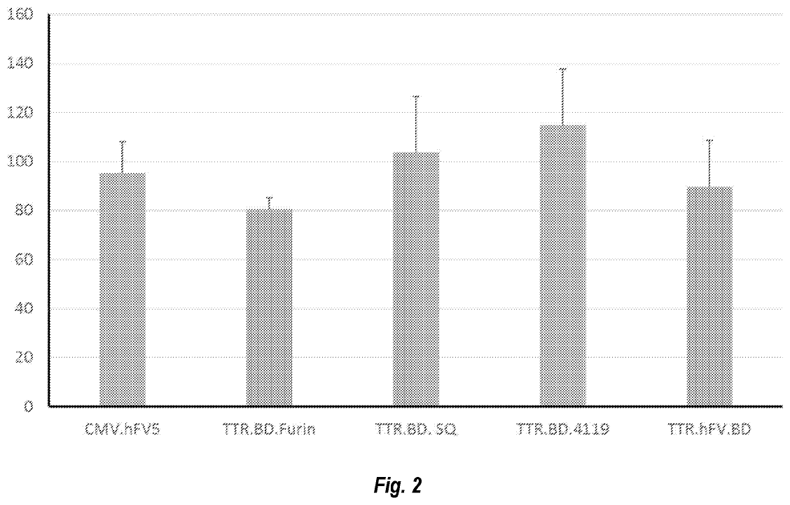

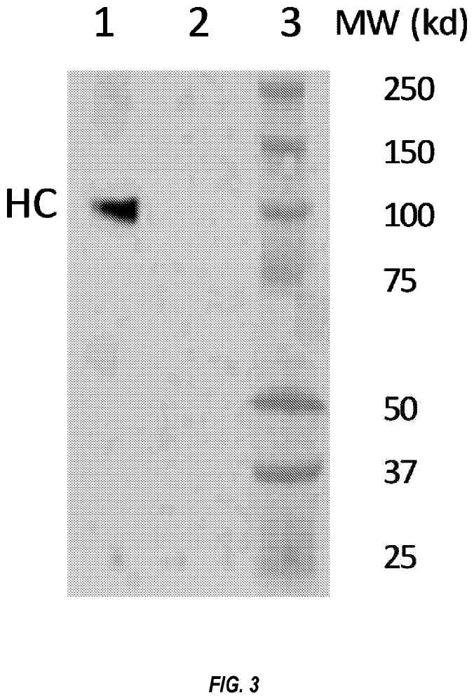

Hemostasis improvement with AAV vector delivery of hFVa. Protein therapy with FVa mutants has been tested for hemophilia with inhibitors. Successful results are achieved in animal models. To explore whether FVa can be delivered by AAV vectors to improve the hemostasis in an animal model with hemophilia, we made several human FV (hFV) cassettes flanked by AAV ITRs with different linker between FV heavy chain (HC) and the light chain (LC) driven by the liver specific promoter TTR (FIG. 1), after hydrodynamic injection of these plasmids into hemophilia A mice, the aPTT analysis was performed at day 2. As shown in FIG. 2, the best result was achieved from the cassette TTR.BD.furin in which the B domain was completely depleted and a furin cleavage motif (RKRRKR) was used to link the HC with LC. Next we transfected the plasmid FVa.BD.furin driven by the CBA promoter into 293 cells, the heavy chain (110 kd) was detected with the antibody GMA-044, which specifically recognizes the hFV HC (FIG. 3). This result indicates that FVa protein can be properly processed and formed by the furin cleavage intracellularly.

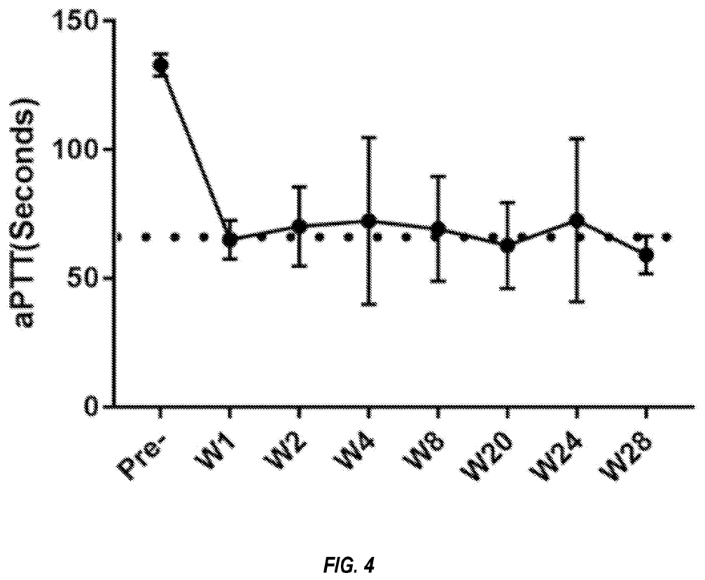

Next, we made AAV8/TTR-hFVa vectors. 1×10¹² particles of AAV8/TTR-hFVa were administered in hemophilia B mice via the tail vein. The complete phenotypic correction was achieved when compared to wt mice over 28 weeks with a normal activated partial thromboplastin time (aPPT) (FIG. 4). This study suggests that utilization of AAV vectors to deliver FVa is safe and maintains the hemostasis.

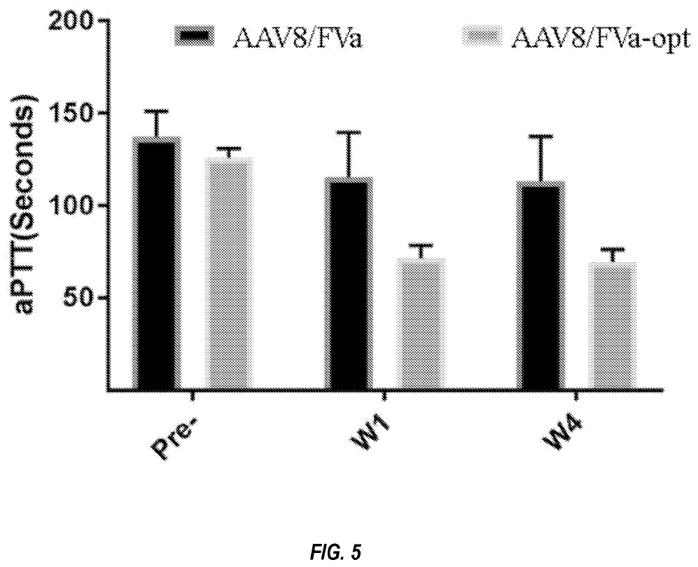

Optimization of FVa codon sequence increases FVa expression. It is known that codon optimization can significantly increase protein expression. For the hFVa cDNA sequence, several sequence elements might inhibit hFVa expression in mammals, including a high frequency of rare codons, a low GC content that could result in decreased mRNA, a cryptic splice donor site, and a RNA instability motif. Optimization of the FVa codon sequence would augment hFVa expression. Utilizing the GenScript codon optimization software, OptimumGene™, a human FVa sequence optimization was designed to increase the GC content from 44 to 55%, and adapted the codon usage for Homo sapiens. We made an AAV8 vector encoding either FVa-opt or FVa driven by the truncated TTR promoter and injected them into hemophilia mice. At week 1 and 4 post AAV administration, blood was collected and the FVa activity was measured using an aPTT analysis. As shown in FIG. 5, a high function of FVa was achieved in mice receiving the AAV8/FVa-opt vectors. This result indicates that the optimization of the FVa codon sequence increases FVa expression and function. Based on these results, we will use the FVa as the template to optimize the AAV cassette in order to further increase FVa expression and its activity by the application of different promoters and linkers between the FVa HC and LC.

In summary, we have generated data from which we can conclude that: (1) the delivery of AAV8 vector encoding human FVa induces a phenotypic correction in hemophilic mice; and (2) optimization of the FVa codon sequence increases FVa expression.

### Example 2: Optimization of AAV/FVa Cassettes for Phenotypic Correction in Hemophilic Mice with Inhibitors

AAV vectors have been successfully used in patients with hemophilia A and hemophilia B. However, this approach is only applied to patients without inhibitors against FVIII or FIX. Although efforts have been focused on the development of FVIIa as a bypass product for treatment of hemophilia with inhibitors, only a suboptimal therapeutic effect has been achieved when a super-physiological dose is used. FVIIa is able to activate FX to generate FXa and then induce thrombin formation. FVa functions as a co-factor of FXa and increases thrombin generation by 10,000 fold, therefore, supplementing the FVa potentially induces more thrombin formation in hemophilia patients with inhibitors. Due to the short half-life of wt FVa, preclinical studies have demonstrated that the treatment with mutant FVa proteins, which are resistant to cleavage by activated protein C (APC), are effective in preventing bleeding in hemophilic animal models. FVa protein therapy is transient and requires repeated infusions. Gene therapy is able to provide long-term transgene expression. However, the DNA constructs encoding FVa mutants may not be suitable for gene therapy delivery since unwanted side effects may be caused from long-term expression of the dys-regulation of mutant FVa.

Gene delivery of wt FVa with AAV vectors has several advantages over FVa mutant protein replacement: (1) AAV vectors have been successfully applied in patients with hemophila A and B and proven safe. (2) Only one infusion is required since long-term transgene expression has been observed in pre-clinical animal models and human clinical trials. (3) There is no contamination from the processes for protein production and purification. (4) There is no need of an extra step to cleave FV using thrombin to generate FVa. (5) The wt FVa will be directly formed after its expression. (6) Its function should be regulated by normal physiological mechanisms. Factor V is synthesized in the liver as a single chain protein. Its N-terminal HC and C-terminal LCs are linked with a large, heavily glycosylated B-domain (domain organization A1-A2-B-A3-C1-C2). Factor V does not have procoagulant activity. It is activated by thrombin via limited proteolysis to release the B domain and the interaction of the HC and the LC generates the procoagulant heterodimer FVa.

Similar to the constructs of FVIII and FVIIa for AAV delivery, we have made the construct (FVa-furin) by using the deletion of the FV B-domain and linked the HC and the LC via a furin cleavage motif. After the delivery of an AAV8 vector encoding FVa-furin into hemophilic mice, complete phenotypic correction was achieved. Although successful in patients with hemophilia in recent clinical trials, there is one concern about capsid specific CTL response. When a high dose of AAV vector is used, the capsid specific CTL response is detected and suggested to eliminate AAV transduced hepatocytes. It has been demonstrated that the capsid antigen presentation in AAV transduced cells is dose-dependent. In spite of encouraging results from the AAV8/FVa-furin vector driven by a weak promoter in a mouse model, it is still necessary to optimize the FVa cassette for a higher expression and then decrease AAV vector dose to avoid strong capsid antigen presentation from the AAV transduced hepatocytes.

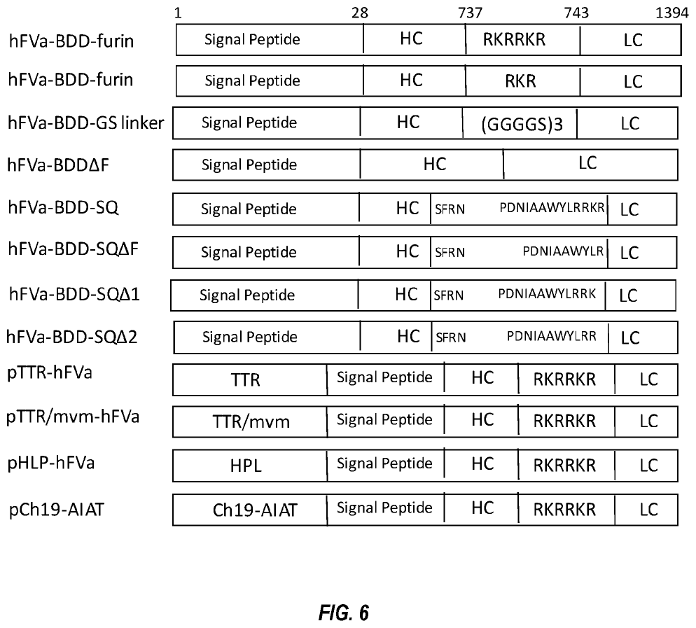

There are several approaches to optimize transgene cassettes for higher expression, including utilization of stronger promoters, and optimization of AAV coding sequences and different linker sequences between the HC and the LC as demonstrated in FVIII. We have made a cassette with FVa coding sequence optimization, and a higher transgene expression was achieved. It has been demonstrated that the linker sequence between the FVIII HC and LC impacts FVIII transgene expression and function. Therefore, the effect of different linker sequences between the HC and the LC on FVa secretion and activity will be examined (FIG. 6). We have demonstrated that small fragments from an AAV2 integration site on human chromosome 19 showed liver specific promoter/enhance function.

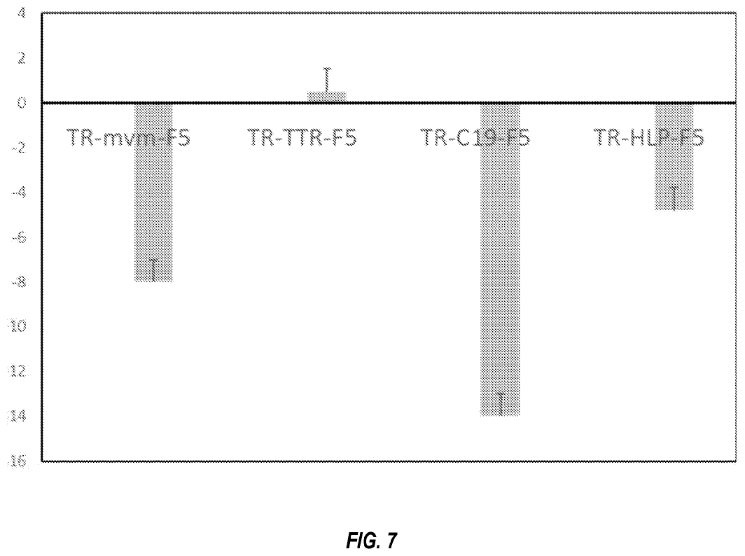

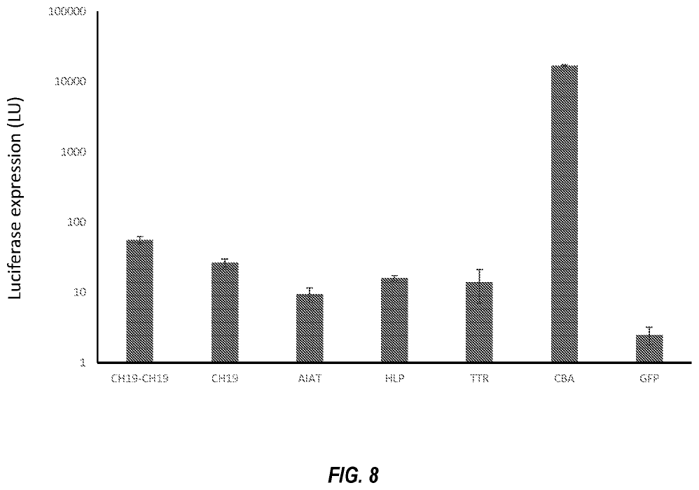

Different liver specific promoters will be designed and their activity on FVa expression will be compared (FIG. 6). When different promoters were used to drive hFVa expression, it was discovered that administration of AAV8/hFVa with the Ch19-AIAT promoter induced much more efficient hemostasis improvement than with other promoters including TTR and HLP, which have been used in clinical trials (FIG. 7). Further study demonstrated that a promoter comprising two copies of Ch19 fragment further increased the promoter function in a liver cell line Huh7 cells (FIG. 8).

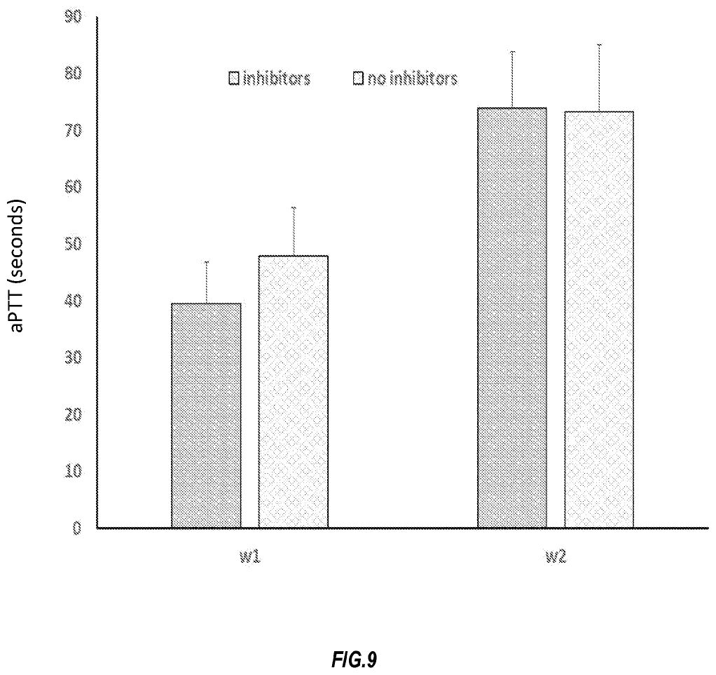

The best hFVa cassette will be packaged in an AAV8 capsid and AAV8/hFVa will be injected into hemophilia mice with inhibitors to study the phenotypic correction. Since hemophilia A (HA) is more common than hemophilia B (HB), and incidence of inhibitor development is higher in HA, we will use HA animal models (mouse and dog) for these proposed studies. As a proof of principle, we have injected AAV8/TTR-hFVa into HA mice, and similar hemostasis improvement was observed between mice with inhibitors and control mice without inhibitors (FIG. 9).

Optimization of the linker sequences between the FVa HC and LC. Recent studies have demonstrated that modifications of furin cleavage motifs can result in increased FVIII expression. Furin processing has been shown to be deleterious to FVIII-SQ secretion and procoagulant activity, and deletion of the furin cleavage site increased FVIII secretion. The cassette FVIII-SQ contains 14 amino acids of the B-domain and the furin recognition site to link the HC and LC of FVIII. Comparable linkers containing the furin recognition motif have been used in the development of hemophilia A therapies. The effect of different linkers between the FVa HC and LC on the FVa expression and function (FIG. 6) will be analyzed.

To study FVa secretion, we will clone different FVa constructs driven by the CBA promoter. After transfection of these FVa cassettes into 293 cells, the supernatant will be analyzed for FVa expression using ELISA and FVa function will be tested with an aPTT assay. For in vivo studies, FVa expression will be driven by the truncated TTR promoter and the FVa cassette is packaged into AAV8 virions. After the systemic administration of AAV8/FVa in HA mice, the plasma will be harvested for FVa expression and will be tested using function assays, including prothrombinase assays, prothrombin time (PT), aPTT, and thrombin generation assays. At the end of the experiments, tail transection will be performed to measure blood loss. When mice are euthanized at end time point, whole blood will be collected for the ROTEM analysis and for detection of inhibitors for hFVa by Bethesda assay.

Clone of FVa cassettes. Routine PCR approaches will be used to amplify target fragments.

Transfection in 293 cells. Different CBA-FVa constructs are transfected into 293 cells, at 48 or 72 hrs, the supernatant is collected and concentrated. FVa expression and function will be analyzed by ELISA and aPTT analysis, respectively.

Production of AAV vectors. All recombinant AAV8 viruses are generated using the standard triple transfection method using the XX6-80 adenoviral helper plasmid with an AAV8 packaging plasmid and an ITR/FVa plasmid.

Systemic administration of AAV8/hFVa in HA mice. AAV8/hFVa vectors will be systemically administered into hemophilia mice at a dose of 5×10¹¹ particles (2.5×10¹³/kg). At indicated time points after AAV injection, blood is harvested for FVa expression and function analysis.

FVa ELISA. The high binding plate is coated with sheep poly-clonal anti-hFV antibody (ab30905, 4 ug/ml). After blocking and incubation with FVa transfected 293 cell supernatant or mouse plasma at different dilutions or standard FV, mouse anti human FV monoclonal antibody (B38, 4 ug/ml) is added, followed by addition of HRP conjugated anti-mouse Ig antibody (1:10000). The color is developed by addition of TNB substrate and stopped by 10% sulfuric acid. The OD value will be read by an ELISA plate reader.

Prothrombinase assays. Prothrombinase assays are performed as described. FVa from 293 cell supernatant or blood is mixed with phospholipid vesicles, and FXa is added, followed by prothrombin, and the reaction is quenched by the addition of HEPES buffered saline. After addition of Pefachrome TH, thrombin formation is assessed by measuring the change in absorbance at 405 nm using a Microplate reader.

aPTT assay. 293 cell supernatant or mouse plasma is mixed with aPTT reagent and incubated at 37° C. Then FVa is added, followed by CaCl2. The clotting times are recorded using an ST4 coagulometer.

PT assay. Supernatant from 293 cells or plasma is mixed with FVa and incubated at 37° C. for 1 min, followed by the addition of Innovin. The clotting times are recorded using an ST4 coagulometer.

hFV Bethesda Unit titre determination. The titer of hFV inhibitors is measured by Bethesda assay. Mouse plasma at different dilutions is incubated with pooled normal human plasma at 37° C. for 2 hours and clotting time is recorded by APTT. Each Bethesda unit corresponds to neutralization of 50% of the factor V clotting activity in standard normal plasma.

Thrombin generation assays. Thrombin generation assays are performed as described. Briefly, 293 supernatant or plasma from AAV8/FVa treated mice, FV or saline is added to human FV-deficient plasma (50% v/v) supplemented with corn trypsin inhibitor, CaCl2, phospholipid vesicles, soluble tissue factor and thrombin substrate Z-Gly-Gly-Arg-AMC. Then the mixture is transferred to a FluoroNunc microtiter plate at 37° C. to monitor fluorescence. Fluorescence time course data are converted into the concentration of thrombin.

Tail bleeding assays. Tail bleeding assays are performed as described. Mice are anesthetized and the distal portion of the tail is cut, and then the tail is immersed in saline for 20 min. Blood loss is determined by measuring the hemoglobin from red blood cells.

Rotational thromboelastometry. Clotting is assessed by rotational thromboelastometry (ROTEM) as described. Briefly, whole blood is collected from the inferior vena cava at sacrifice, mixed at a ratio of 9:1 with 3.2% sodium citrate, and then the mixture is coagulated with 20 μL of 0.2 M CaCl2 in a pre-warmed rotational thromboelastometer cup.

Exploration of a stronger promoter for FVa expression. We will clone a hybrid promoter containing a chr19 small fragment and the AAT AIAT promoter, and then examine its liver specific FVa expression in HA mice when compared to that of other liver specific promoters: TTR, TTR-MVM, and HLP. After administration of AAV8/FVa driven by different promoters, analysis of the transgene FVa expression and its function will be performed as described herein. At the end of the experiments, the mice will be euthanized, and liver tissue DNA and RNA will be extracted for AAV genome copy number and transcription analyses, respectively.

Animal study in HA mice. 5×10¹¹ particles of AAV8/FVa driven by different promoters will be administered into HA mice via systemic injection. At indicated time points after AAV injection, blood is harvested for FVa expression and functional analysis. At the end of the study, mouse liver will be harvested for DNA and RNA. AAV genome copy number and FVa transcription will be analyzed using Q-PCR.

Q-PCR. Q-PCR is performed on genomic DNAs or cDNA isolated from mice liver using DyNAmo HS SYBR Green qPCR Kit. The copy number of hFVa DNA is quantified against a standard generated with linearized plasmid FVa serially diluted in pooled genomic DNAs from naive C57 mice. Real-time PCR is performed using a LightCycler 480 instrument (Roche). All samples are normalized for mouse β-actin.

RNA extraction and cDNA synthesis. RNA from liver tissues is isolated using TRIzol Reagent (Invitrogen). Synthesis of first strand cDNA from RNA templates is performed using RevertAid First Strand cDNA Synthesis Kit (Thermo Fisher Scientific).

Animal study in HB mice. 1×10¹¹ particles of AAV8/hFVa-opt driven by different promoters were administered into hemophilia B mice via tail vein. At pre and week 8 post AAV injections, blood was harvested for coagulation assay. The percentage of clot time change for APTT at week 8 post AAV administrations was calculated while compared to APTT time pre-AAV injection (FIG. 5)

Phenotypic correction of hemophilia in HA mice with inhibitors. Hemophilia A mice will be immunized with the recombinant coagulation factor FVIII for inhibitor generation. AAV8/hFVa optimized as described herein will be administered. The hemostasis will be evaluated as described.

Animal experiment. Inhibitors are induced by administration of rFVIII (100 IU/kg) intravenously via retro-orbital vein plexus in HA mice weekly for a total of 5 doses. Citrated blood will be collected by retro-orbital plexus. FVIII inhibitor titer will be measured based on Bethesda assay. One week later after last boost of rFVIII, 5×10¹¹ particles of AAV8/FVa will be administered via tail vein injection. At indicated time points, after AAV injection, blood is harvested for FVa expression and function analysis. At the end of the study, hemostasis will also be evaluated as described herein.

FVIII inhibitor detection. Inhibitors for hFVIII are measured using the Bethesda assay. Mouse plasma is serially diluted and mixed 1:1 with pooled normal human plasma, and incubated for 2 hours at 37° C. The remaining FVIII activity is quantified by aPTT assay.

Our preliminary results have demonstrated that hFVa can be generated by the deletion of the B-domain and by using a furin cleavage site to link the FV HC and LC. After the delivery of AAV8/hFVa driven by a weak TTR promoter into hemophilia mice, complete phenotypic correction was achieved. Our previous studies have demonstrated that the TTR promoter with a mvm intron dramatically increases FIX expression when compared to that of other tested promoters and similar to the HLP promoter. The addition of small ch19 fragment to the upstream of the miniCMV promoter induced liver specific transduction enhancement which is higher than the TTR promoter with a mvm intron. The delivery of hFVa cassette with the optimized linker between FV HC and LC driven by a Ch19-AIAT promoter via AAV8 vectors should induce a high FVa expression and phenotypic correction in hemophilia mice with inhibitors with similar efficiency to that in hemophilia mice without inhibitors.

### Example 3: Investigation of the Synergistic Effect from Combinational AAV Gene Delivery of FVa and FVIIa

The coagulation cascade of hemostasis has two initial pathways which lead to fibrin formation: the contact activation pathway, and the tissue factor pathway. For the tissue factor pathway, after blood vessel damage, FVII interacts with tissue factor (TF) from tissue-factor-expressing cells to form an activated complex (TF-FVIIa). Then TF-FVIIa activates FX to FXa following the common pathway. In the final common pathway, FXa and its co-factor FVa form the prothrombinase complex, which activates prothrombin to thrombin. In hemophilia patients, due to deficiency of FVIII and FIX, the contact activation pathway doesn't function, so the factors (bypass product) involved in the tissue pathway and final common pathway can be used as an alternative approach, especially in patients with inhibitors. Although great success has been achieved with FVIIa in clinical trials in patients with inhibitors, the extra-high dose of FVIIa is needed and only sup-optimal effect has been obtained. Even with high-dose of AAV vector for delivery of FVIIa in a double-stranded (ds) template and driven by the TTR promoter with mvm intron, no complete correction of coagulation was observed in animal models. These results strongly suggest that enhanced FVIIa expression in blood is not sufficient to convert FX to FXa, which may also explain why hemophilia patients still have the bleeding phenotype even though the alternative tissue factor pathway of the coagulation cascade involving FVIIa is intact.

We have shown that FVa delivered by single-stranded (ss) AAV vector, which is 10-20 fold lower transduction than dsAAV, was able to completely correct the phenotype of hemophilia in hemophilic mice even when a weak liver specific promoter TTR was used. This result suggests that FVa delivered by an AAV vector may result in much better hemostasis than AAV/FVIIa. It is important to elucidate the therapeutic efficiency of FVIIa and FVa delivered by AAV vectors for future effective selection. Since FVIIa and FVa use different mechanisms for coagulation, and it has been reported that the combination of FVa and FVIIa protein replacement had a synergistic effect. We hypothesize that the combination of FVa and FVIIa delivered with AAV vectors will induce a stronger hemostatic response in hemophilia with inhibitors, and therefore the total dose of AAV vectors will be reduced to achieve a therapeutic effect. This would decrease the capsid antigen presentation on AAV transduced hepatocytes and lower the labor force to make these vectors. Hence, herein, we will first compare the hemostasis effect of FVa and FVIIa with different doses of AAV vectors and investigate the complications from the super-dose of AAV8/FVa after long-term transduction. Next, we will design a different combination of AAV/FVa and AAV/FVIIa to explore the best combination for maximum hemostasis in hemophilia mice with inhibitors.

Comparison of the hemostasis effect of FVa to FVIIa via AAV8 mediated delivery. The same promoter described herein will be used to drive FVa or FVIIa expression. Since hFVIIa does not efficiently function in mice, we will compare the effect of mouse FVa (mFVa) with mouse FVIIa (mFVIIa) on hemostasis. Due to the size difference of mFVa (1356 bp) and mFVIIa cDNA (4164 bp), ds mFVa and ssFVa cassettes will be used for AAV vector production. Although a dsAAV vector induces much higher (10-20 fold) transduction than ssAAV vectors, the main focus of this study is to compare their hemostasis at the same setting (the promoter, and polyA), so the same dose of AAV8 vector for mFVa or mFVIIa will be applied. After the administration of ssAAV8/mFVa or scAAV8/mFVIIa at different doses into hemophilia mice, the phenotypic correction will be monitored as described above. Also, a long-term follow up will be carried out to evaluate mouse survival rate and thrombosis risk, especially in mice with the high-dose of AAV8/FVa and AAV8/FVIIa. In addition to the necropsy evaluations for evidence of thrombosis from all tissues and organs, the potential for high FVa or FVIIa expression to lead to inappropriate activation of coagulation will be assessed by measuring the plasma thrombin-antithrombin (TAT) complexes, d-dimer, and prothrombin fragment 1+2. To avoid the immune response to hFVa, mouse FVa (mFVa) will be used.

Construction of murine FVa. Mouse FV is composed of a signal peptide (aa1-19), the heavy chain (20-736), B domain (aa 737-1533) and the light chain (aa1534-2183). Based on the information described herein, the optimized promoter and linker will be used to make mFVa construct.

Animal experiments. HA mice will receive ssAAV8/mFVa or scAAV8/mFVIIa at the following doses: 1×10¹¹/kg, 3×10¹¹/kg, 1×10¹²/kg, 3×10¹²/kg, 1×10¹³/kg, 3×10¹³/kg, 1×10¹⁴/kg, 3×10¹⁴/kg and 1×10¹⁵/kg. At indicated time points, plasma will harvested for hemostasis analysis. At one year after administration of AAV vectors, mice will be euthanized for evaluation of hemostasis and thrombosis.

ELISA for mFVIIa expression. For the quantification of mFVIIa expression in mouse plasma, ELISA is used as described.

Histopathological examination at necropsy of hemophilic mice. At the time of sacrifice of hemophilic mice after administration of AAV8/mFVa or AAV8/mFVIIa vectors, mice are sacrificed by CO₂ asphyxiation and examined for gross signs of hemorrhage. All tissues are immersion-fixed in 10% neutral buffered formalin, trimmed, processed, sectioned, and stained with hematoxylin and eosin (H&E) by routine methods, and a panel of organs and tissues is evaluated microscopically for histopathological changes. Heart, lung, liver, spleen, kidney, and brain are evaluated for the presence of fibrosis and/or microvascular thrombus formation by immunohistochemistry for fibrinogen, and additional evaluation with Masson's trichrome and phosphotungstic acid hematoxylin for collagen.

Thrombin/antithrombin III assay. Thrombin-antithrombin complexes (TAT) form covalently following thrombin generation and have a plasma half-life of 10 to 15 minutes. The presence of TAT indicates ongoing thrombin formation and the consumption of antithrombin. Upon activation of coagulation, antithrombin complexes with thrombin as well as other serine proteases. This binding of antithrombin with thrombin results in complete inhibition of thrombin's activity. Elevated levels of TAT may be associated with disseminated intravascular coagulation and other predisposing causes of thrombosis. The TAT assay can detect the intravascular generation of thrombin and provides valuable information in the diagnosis of thrombotic events. TAT complexes are measured from platelet-poor citrated plasma collected as a terminal puncture of the inferior vena cava at the end of the study, using an Enzygnost TAT micro ELISA system (Siemens Healthcare Diagnostics, Tarrytown, N.Y.).

D-dimer detection. D-dimer is a protein formed by the cross-linking of two D fragments of the fibrin protein. D-dimer is one of several fibrin degradation products (FDPs) formed by the degradation of a blood clot by fibrinolysis. Its measurement is used to diagnose thrombosis. D-dimer is detected by ELSIA.

Measurement of prothrombin fragment 1+2. Prothrombin fragment 1+2 has also been used to diagnose thrombosis in clinics. ELISA kit will be used for detection of prothrombin fragment 1+2.

Investigation of the effect of the combination of AAV vector encoding FVa and FVIIa on hemostasis in HA mice with inhibitors. To study the effect of the combination of FVa with FVIIa delivered by AAV vectors, based on the results from studies described herein, the sub-optimal dose of AAV vector for either FVa or FVIIa will be chosen. The experiments will be designed as follows: a fixed sub-optimal dose of AAV8/FVa is mixed with different doses of AAV8/FVIIa, which are lower than the dose to achieve maximum function; a fixed sub-optimal dose of AAV8/FVIIa is mixed with different doses of AAV8/FVa; the same dose of individual AAV8/FVa or FVIIa as the total dose from the mixture. After the systemic administration of the mixtures or individual vector, hemostasis will be evaluated as described above, including transgene expression, APTT, PT, thrombin generation assay, ROTEM analysis, tail bleeding assay, TAT assay, D-dimer, Prothrombin fragment 1+2, and histopathological examination.

Animal experiment. Hemophilia A mice are treated with rhFVIII to induce inhibitors and then receive AAV vector with the mixtures of AAV8/mFVa and AAV8/mFVIIa at different ratios via tail vein injection. As control, the same dose of AAV8/FVa or AAV8/mFVIIa as the mixture will be used for comparison. At indicated time points, blood will be collected for transgene expression and functional analysis of hemostasis and thrombosis. At the end of experiments, mice will be evaluated by tail bleeding. Blood and different tissues will be collected for ROTEM analysis and histopathological examination.

In previous studies, AAV9 induced a similar liver transduction to AAV8 in mice. When the high-dose of the AAV9 vector was used to deliver mFVIIa driven by the TTR promoter with a mvm intron in a double-stranded template in hemophilia mice, the therapeutic effect was achieved, but the correction was not close to that in wild type mice. A similar dose of the AAV8 vector was applied to deliver hFVa driven by the truncated TTR promoter without the mvm intron in a single-stranded cassette, when compared to that of wild mice, a complete phenotypic correction was observed in hemophilic mice. It is well known that dsAAV vector induces much higher transduction than a ssAAV vector and the TTR promoter with a mvm intron results in a stronger transgene expression than that of the truncated TTR promoter.

The combination of AAV8/FVa and AAV8/FVIIa should significantly improve hemostasis in hemophilia mice and induce better phenotypic correction when compared to either AAV8/mFVa or AAV8/mFVIIa alone, when the same dose of the AAV8 vectors is used. Different combinations of AAV8/mFVa and AAV8/mFVIIa may result in different efficiencies for hemostasis. The combination should induce much better hemostasis than others. This combination should achieve an improved correction of disease phenotype in hemophilia mice with inhibitors.

### Example 4: Study of the Phenotypic Correction in Hemophilic Dogs with Inhibitors Using AAV8 Vectors Encoding FVa Alone or in Combination with FVIIa

The advancement in molecular medicine relies on the availability of well-characterized animal models. Studies in these animals represent the important steps of translational research to develop better and safer treatments. Regarding hemophilia, murine models have been used for studies of large groups of animals; however, canine models are important for testing scale-up and for long-term follow-up as well as characterizing the immune response to hemophilic factors and gene delivery vectors. The hemophilia A canine model from the colony at the University of North Carolina at Chapel Hill is characterized by the presence of an intron 22 inversion, resulting in the complete absence of FVIII activity in plasma and produces a severe human-like hemophilia.

Previous work has demonstrated that administration of an AAV vector encoding canine FVIIa resulted in the following therapeutic effects: (1) long-term expression of cFVIIa, (2) shortened prothrombin time, (3) partial correction of the whole blood clotting time and thromboelastography parameters, (4) a complete absence of spontaneous bleeding episodes, and (5) no evidence of hepatotoxicity and thrombotic complications. Based on primary results from hemophilic mouse experiments, FVa delivered by an AAV vector may induce more improved hemostasis than FVIIa. We presume that the improved hemostasis from AAV vector mediated canine FVa delivery will be achieved in hemophilia A dogs, and that the combination of AAV/FVa and AAV/FVIIa will show a synergistic effect. Therefore, we will study hemostasis improvement after the administration of either AAV8/cFVa alone or in combination with AAV8/CFVIIa in hemophilia A dogs with inhibitors.

Study the effect of cFVa delivered by AAV vectors on phenotypic correction in hemophilia A dogs. Based on the information from Examples 2 and 3, to avoid the immune response and FV species specific activity, we will first make a canine FVa (cFVa) construct which is packaged into AAV8 virions. To test the function of cFVa, since preliminary results showed the human FVa function in mice, we will first inject AAV8/cFVa into hemophilia mice and examine cFVa function for phenotypic correction. It has been demonstrated that similar transduction efficiency in primates can be achieved by using 10 more fold vector dose than that used in hemophilia mouse models with AAV/FIX gene delivery. To study the effect of AAV8/cFVa on hemostasis in hemophilic dogs, we will scale up the administration dose of AAV8/cFVa by 10 more fold higher than that for the mouse model.

In addition, to compare whether the inhibitors to FVIII impact the effect of cFVa, we will design two groups: hemophilia A dogs with or without FVIII inhibitors. After the administration of AAV8/cFVa via peripheral vein injection, the cFVa expression and functional assay will be performed including the whole blood clotting time (WBCT), aPTT, TAT, TEG, TAT, d-dimer and prothrombin Fragment 1+2.

Construction of canine FVa. Canine FV has two variants and the variant X1 is composed of the signal peptide (aa1-31), the heavy chain (aa 32-741), B domain (aa742-1557) and the light chain (aa1558-2208), the variant X2 contains the heavy chain (aa32-737), B domain (aa 738-1571) and the light chain (aa1572-2222). For these studies, we will make a cFVa construct driven from FV variant X1.

Mouse experiment. 5×10¹¹ particles of AAV8/cFVa vectors will be administered into hemophilia mice, and at different time points, blood will be collected for cFVa expression and function analysis as described above.

FVIII inhibitor induction in hemophilia A dogs. Dogs are challenged with 0.5 mg of pooled plasma-derived, purified cFVIII concentrate (Enzyme Research Laboratory, South Bend, Ind.) by intravenous injection. Humoral responses to cFVIII are monitored using Bethesda assay.

Gene Delivery in hemophilia A dogs. The hemophilia A dogs, screened negative for AAV8 Nabs, will be treated with rAAV8/cFVa via cephalic vein at 9 weeks of age (4.5 kg). Blood will be collected and coagulation assays will be performed at indicated time points. At one year after virus administration, the animal will be euthanized with intravenous pentobarbital overdose and tissues will be collected for histologic evaluation. Two groups will be designed: dogs without cFVIII inhibitors and dogs with cFVIII inhibitors.

Investigation of the effect of the combination of cFVa and cFVIIa on hemostasis in hemophilia dogs with inhibitors. Based on the results in hemophilia mice, a mixture of AAV8/cFVa and AAV8/cFVIIa at the same ratio as in mice will be administrated into the hemophilia A dogs with FVIII inhibitors. The phenotypic correction will be monitored at the indicated time points. Three groups will be designed: AAV8/cFVa, AAV8/cFVIIa, and AAV8/cFVa in combination with AAV8/cFVIIa. All dogs will receive the same dose of the AAV8 vectors.

Dog experiment. Hemophilia A dogs without neutralized antibodies to AAV8 will be challenged with cFVIII for inhibitor generation and will then receive the same dose of AAV8/cFVa or AAV8/cFVIIa or the combination of AAV8/cFVa with AAV8/cFVIIa via peripheral vein injection. At different time points after AAV administration, the phenotypic correction will be analyzed.

The administration of AAV8/cFVa should induce canine FVa expression and improve hemostasis in hemophilia dogs regardless of cFVIII inhibitor existence. It is anticipated that improved phenotypic correction can be achieved if the combination of AAV8/cFVa with AAV8/cFVIIa is administered compared to AAV8/cFVa or AAV8/cFVIIa alone.

While there are shown and described particular embodiments of the invention, it is to be understood that the invention is not limited thereto but may be otherwise variously embodied and practiced within the scope of the following claims. Since numerous modifications and alternative embodiments of the present invention will be readily apparent to those skilled in the art, this description is to be construed as illustrative only and is for the purpose of teaching those skilled in the art the best mode for carrying out the present invention. Accordingly, all suitable modifications and equivalents may be considered to fall within the scope of the following claims.

TABLE 1

| AAV Serotypes/Isolates | GenBank Accession Number |
|---|---|
| Clonal Isolates | |
| Avian AAV ATCC VR-865 | AY186198, AY629583, NC_004828 |
| Avian AAV strain DA-1 | NC_006263, AY629583 |
| Bovine AAV | NC_005889, AY388617 |
| AAV4 | NC_001829 |
| AAV5 | AY18065, AF085716 |
| Rh34 | AY243001 |
| Rh33 | AY243002 |
| Rh32 | AY243003 |
| AAV10 | AY631965 |
| AAV11 | AY631966 |
| AAV12 | DQ813647 |
| AAV13 | EU285562 |
| Clade A | |
| AAV1 | NC_002077, AF063497 |
| AAV6 | NC_001862 |
| Hu.48 | AY530611 |
| Hu 43 | AY530606 |
| Hu 44 | AY530607 |
| Hu 46 | AY530609 |
| Clade B | |
| Hu19 | AY530584 |
| Hu20 | AY530586 |
| Hu23 | AY530589 |
| Hu22 | AY530588 |
| Hu24 | AY530590 |
| Hu21 | AY530587 |
| Hu27 | AY530592 |
| Hu28 | AY530593 |
| Hu29 | AY530594 |
| Hu63 | AY530624 |
| Hu64 | AY530625 |
| Hu13 | AY530578 |
| Hu56 | AY530618 |
| Hu57 | AY530619 |
| Hu49 | AY530612 |
| Hu58 | AY530620 |
| Hu34 | AY530598 |
| Hu35 | AY530599 |
| AAV2 | NC_001401 |
| Hu45 | AY530608 |
| Hu47 | AY530610 |
| Hu51 | AY530613 |
| Hu52 | AY530614 |
| Hu T41 | AY695378 |
| Hu S17 | AY695376 |
| Hu T88 | AY695375 |
| Hu T71 | AY695374 |
| Hu T70 | AY695373 |
| Hu T40 | AY695372 |
| Hu T32 | AY695371 |
| Hu T17 | AY695370 |
| Hu LG15 | AY695377 |
| Clade C | |
| AAV 3 | NC_001729 |
| AAV 3B | NC_001863 |
| Hu9 | AY530629 |
| Hu10 | AY530576 |
| Hu11 | AY530577 |
| Hu53 | AY530615 |
| Hu55 | AY530617 |
| Hu54 | AY530616 |
| Hu7 | AY530628 |
| Hu18 | AY530583 |
| Hu15 | AY530580 |
| Hu16 | AY530581 |
| Hu25 | AY530591 |
| Hu60 | AY530622 |
| Ch5 | AY243021 |
| Hu3 | AY530595 |
| Hu1 | AY530575 |
| Hu4 | AY530602 |
| Hu2 | AY530585 |
| Hu61 | AY530623 |
| Clade D | |
| Rh62 | AY530573 |
| Rh48 | AY530561 |
| Rh54 | AY530567 |
| Rh55 | AY530568 |
| Cy2 | AY243020 |
| AAV7 | AF513851 |
| Rh35 | AY243000 |
| Rh37 | AY242998 |
| Rh36 | AY242999 |
| Cy6 | AY243016 |
| Cy4 | AY243018 |
| Cy3 | AY243019 |
| Cy5 | AY243017 |
| Rh13 | AY243013 |
| Clade E | |
| Rh38 | AY530558 |
| Hu66 | AY530626 |
| Hu42 | AY530605 |
| Hu67 | AY530627 |
| Hu40 | AY530603 |
| Hu41 | AY530604 |
| Hu37 | AY530600 |
| Rh40 | AY530559 |
| Rh2 | AY243007 |
| Bb1 | AY243023 |
| Bb2 | AY243022 |
| Rh10 | AY243015 |
| Hu17 | AY530582 |
| Hu6 | AY530621 |
| Rh25 | AY530557 |
| Pi2 | AY530554 |
| Pi1 | AY530553 |
| Pi3 | AY530555 |
| Rh57 | AY530569 |
| Rh50 | AY530563 |
| Rh49 | AY530562 |
| Hu39 | AY530601 |
| Rh58 | AY530570 |
| Rh61 | AY530572 |
| Rh52 | AY530565 |
| Rh53 | AY530566 |
| Rh51 | AY530564 |
| Rh64 | AY530574 |
| Rh43 | AY530560 |
| AAV8 | AF513852 |
| Rh8 | AY242997 |
| Rh1 | AY530556 |
| Clade F | |
| AAV9 (Hu14) | AY530579 |
| Hu31 | AY530596 |
| Hu32 | AY530597 |

TABLE 2
Amino acid residues and abbreviations

| | Abbreviation | |
|---|---|---|
| Amino Acid Residue | Three-Letter Code | One-Letter Code |
| Alanine | Ala | A |
| Arginine | Arg | R |
| Asparagine | Asn | N |
| Aspartic acid (Aspartate) | Asp | D |
| Cysteine | Cys | C |
| Glutamine | Gln | Q |
| Glutamic acid (Glutamate) | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

TABLE 3

| Serotype | Position 1 | Position 2 |
|---|---|---|
| AAV1 | A263X | T265X |
| AAV2 | Q263X | −265X |
| AAV3a | Q263X | −265X |
| AAV3b | Q263X | −265X |
| AAV4 | S257X | −259X |
| AAV5 | G253X | V255X |
| AAV6 | A263X | T265X |
| AAV7 | E264X | A266X |
| AAV8 | G264X | S266X |
| AAV9 | S263X | S265X |

Where, (X) → mutation to any amino acid
(−) → insertion of any amino acid
Note:
Position 2 inserts are indicated by the site of insertion

TABLE 4

| | |
|---|---|
| Modified Amino Acid Residue | Abbreviation |

Amino Acid Residue Derivatives

| | |
|---|---|
| 2-Aminoadipic acid | Aad |
| 3-Aminoadipic acid | bAad |
| beta-Alanine, beta-Aminoproprionic acid | bAla |
| 2-Aminobutyric acid | Abu |
| 4-Aminobutyric acid, Piperidinic acid | 4Abu |
| 6-Aminocaproic acid | Acp |
| 2-Aminoheptanoic acid | Ahe |
| 2-Aminoisobutyric acid | Aib |
| 3-Aminoisobutyric acid | bAib |
| 2-Aminopimelic acid | Apm |
| t-butylalanine | t-BuA |
| Citrulline | Cit |
| Cyclohexylalanine | Cha |
| 2,4-Diaminobutyric acid | Dbu |
| Desmosine | Des |
| 2,2′-Diaminopimelic acid | Dpm |
| 2,3-Diaminoproprionic acid | Dpr |
| N-Ethylglycine | EtGly |
| N-Ethylasparagine | EtAsn |
| Homoarginine | hArg |
| Homocysteine | hCys |
| Homoserine | hSer |
| Hydroxylysine | Hyl |
| Allo-Hydroxylysine | aHyl |
| 3-Hydroxyproline | 3Hyp |
| 4-Hydroxyproline | 4Hyp |
| Isodesmosine | Ide |
| allo-Isoleucine | aIle |
| Methionine sulfoxide | MSO |
| N-Methylglycine, sarcosine | MeGly |
| N-Methylisoleucine | MeIle |
| 6-N-Methyllysine | MeLys |
| N-Methylvaline | MeVal |
| 2-Naphthylalanine | 2-Nal |
| Norvaline | Nva |
| Norleucine | Nle |
| Ornithine | Orn |
| 4-Chlorophenylalanine | Phe(4-Cl) |
| 2-Fluorophenylalanine | Phe(2-F) |
| 3-Fluorophenylalanine | Phe(3-F) |
| 4-Fluorophenylalanine | Phe(4-F) |
| Phenylglycine | Phg |
| Beta-2-thienylalanine | Thi |

1. A synthetic protein molecule, comprising:
a) a signal peptide;
b) a factor Va (FVa) heavy chain comprising the amino acid sequence of SEQ ID NO:2;
c) a linker sequence; and
d) a FVa light chain comprising the amino acid sequence of SEQ ID NO:3, with the proviso that the synthetic protein molecule does not include a FVa B domain.

2. The synthetic protein molecule of claim 1, wherein the signal peptide comprises an amino acid sequence selected from the group consisting of:
hFV: MFPGCPRLWVLVVLGTSWVGWGSQGTEA (SEQ ID NO:1);
hFVII: MVSQALRLLCLLLGLQGCLA (SEQ ID NO:6);
hFIX: MQRVNMIMAESPGLITICLLGYLLSAEC (SEQ ID NO:7);
hFVIII: MQIELSTCFFLCLLRFCFS (SEQ ID NO:8);
Human fibrinogen-alpha chain: MFSMRIVCLVLSVVGTAWT (SEQ ID NO:9);
Human fibrinogen-beta chain: MKRMVSWSFHKLKTMKHLLLLLLCVFLVKS (SEQ ID NO:10);
Human fibrinogen-gamma chain: MSWSLHPRNLILYFYALLFLSSTCVA (SEQ ID NO:11);
hFXII: MRALLLLGFLLVSLESTLS (SEQ ID NO:12);
Protein C: MWQLTSLLLFVATWGISG (SEQ ID NO:13);
Protein S: MRVLGGRCGALLACLLLVLPVSEA (SEQ ID NO:14);
Thrombin: MAHVRGLQLPGCLALAALCSLVHS (SEQ ID NO:15);
Anti-thrombin: MYSNVIGTVTSGKRKVYLLSLLLIGFWDCVTC (SEQ ID NO:16);
Serum albumin: MKWVTFISLLFLFSSAYS (SEQ ID NO:17);
Transferrin: MRLAVGALLVCAVLGLCLA (SEQ ID NO:18);
Alpha-1 antitrypsin: MPSSVSWGILLLAGLCCLVPVSLA (SEQ ID NO:19);
Fibronectin: MLRGPGPGLLLLAVQCLGTAVPSTGASKSKR (SEQ ID NO:20);
Alpha-1-microglobulin: MRSLGALLLLLSACLAVSA (SEQ ID NO:21);
Alpha 1-antichymotrypsin: MERMLPLLALGLLAAGFCPAVLC (SEQ ID NO:22);
Apo A: MKAAVLTLAVLFLTGSQA (SEQ ID NO:23);
Apo B: MDPPRPALLALLALPALLLLLLAGARA (SEQ ID NO:24);
Apo E: MKVLWAALLVTFLAGCQA (SEQ ID NO:25);
Alpha-fetoprotein: MKWVESIFLIFLLNFTES (SEQ ID NO:26);
C-reactive protein: MEKLLCFLVLTSLSHAFG (SEQ ID NO:27);
Plasminogen: MEHKEVVLLLLLFLKSGQG (SEQ ID NO:28);
Ceruloplasmin: MKILILGIFLFLCSTPAWA (SEQ ID NO:29);
Complement C1q subunit A: MEGPRGWLVLCVLAISLASMVT (SEQ ID NO:30);
Complement C2: MGPLMVLFCLLFLYPGLADS (SEQ ID NO:31);
Complement C3: MGPTSGPSLLLLLLTHLPLALG (SEQ ID NO:32);
Complement C4A: MRLLWGLIWASSFFTLSLQ (SEQ ID NO:33);
Complement C5: MGLLGILCFLIFLGKTWG (SEQ ID NO:34);
Complement C6: MARRSVLYFILLNALINKGQA (SEQ ID NO:35);
Complement C7: MKVISLFILVGFIGEFQSFSSA (SEQ ID NO:36);
Complement CBA: MFAVVFFILSLMTCQPGVTA (SEQ ID NO:37);
Complement C9: MSACRSFAVAICILEISILTA (SEQ ID NO:38);
α2-antiplasmin: MALLWGLLVLSWSCLQGPCSVFSPVSA (SEQ ID NO:39);
Transcortin: MPLLLYTCLLWLPTSGLWTVQA (SEQ ID NO:40);
Haptoglobin: MSALGAVIALLLWGQLFA (SEQ ID NO:41);
Hemopexin: MARVLGAPVALGLWSLCWSLAIA (SEQ ID NO:42);
IGF binding protein 1: MSEVPVARVWLVLLLLTVQVGVTAG (IGFBP2-7) (SEQ ID NO:43);
Transthyretin: MASHRLLLLCLAGLVFVSEA (SEQ ID NO:44);
Insulin-like growth factor 1 (IGF-1): MGKISSLPTQLFKCCFCDFLK (SEQ ID NO:45);
Thrombopoietin: MELTELLLVVMLLLTARLTLS (SEQ ID NO:46);
β2 microglobulin: MSRSVALAVLALLSLSGLEA (SEQ ID NO:47);
alpha-2-Macroglobulin: MGKNKLLHPSLVLLLLVLLPTDA (SEQ ID NO:48);
and any combination thereof.

3. The synthetic protein molecule of claim 1, wherein the linker sequence comprises an amino acid sequence selected from a furin cleavage motif (RKRRKR) (SEQ ID NO:49); a 2A peptide, a protein linker comprising the formula (GGGGS)ₙ, or (GS)ₙ; a snake B domain; a human FV B domain N-terminus within 100 amino acids; a human FV B domain C-terminus within 100 amino acids; a human FVIII B domain N-terminus within 100 amino acids; a human FVIII B domain C-terminus within 100 amino acids; and any combination thereof.

4. A nucleic acid molecule comprising a nucleotide sequence that encodes the synthetic protein molecule of claim 1.

5. The nucleic acid molecule of claim 4, comprising a nucleotide sequence that has been optimized to increase expression of the nucleotide sequence relative to a nucleotide sequence that has not been optimized.

6. The nucleic acid molecule of claim 4, further comprising a promoter sequence.

7. A recombinant nucleic acid construct, comprising the nucleic acid molecule of claim 4.

8. A recombinant nucleic acid molecule, comprising an adeno-associated virus (AAV) 5′ inverted terminal repeat (ITR), the nucleic acid molecule claim 4 operably linked to a promoter, and an AAV 3′ ITR.

9. An AAV particle comprising the nucleic acid molecule of claim 4.

10. A recombinant nucleic acid molecule comprising a lentivirus 5′ long terminal repeat (LTR), the nucleic acid molecule of claim 4 operably linked to a promoter, and a lentivirus 3′ LTR.

11. A lentivirus particle, comprising the nucleic acid molecule of claim 4.

12. A recombinant nucleic acid molecule comprising an adenovirus (Ad) 5′ ITR, the nucleic acid molecule of claim 4 operably linked to a promoter, and an AAV 3′ ITR.

13. An Ad particle, comprising the nucleic acid molecule of claim 4.

14. The nucleic acid molecule of claim 6, wherein the promoter sequence is the nucleotide sequence:
tctggcgatttccactgggcgcctcggagagcggacttcccagtgtgcatcggggcacagcgactcctggaagtggccaagggccactt ctgctaatggactccatttcccagcgctccccagatctgggcgactcagatcccagccagtggacttagcccctgtttgctcctccgataact ggggtgaccttggttaatattcaccagcagcctcccccgttgcccctctggatccactgcttaaatacggacgaggacagggccctgtctcct cagcttcaggcaccaccactgacctgggacagtgaatc (SEQ ID NO:56), or the nucleotide sequence: tctggcgatttccactgggcgcctcggagagcggacttcccagtgtgcatcggggcacagcgactcctggaagtggccaagggccactt ctgctaatggactccatttcccagcgctcccc (SEQ ID NO:54), operably linked to the nucleotide sequence: ggcgactcagatcccagccagtggacttagcccctgtttgctcctccgataactggggtgaccttggttaatattcaccagcagcctcccccg ttgcccctctggatccactgcttaaatacggacgaggacagggccctgtctcctcagcttcaggcaccaccactgacctgggacagtgaatc (SEQ ID NO:55).

15. A plasmid comprising the nucleic acid molecule of claim 4.

16. (canceled)

17. A recombinant nucleic acid molecule comprising the nucleotide sequence of SEQ ID NO: 50.

18-21. (canceled)

22. A recombinant nucleic acid construct comprising the recombinant nucleic acid molecule of claim 17.

23. A recombinant nucleic acid molecule comprising an adeno-associated virus (AAV) 5′ inverted terminal repeat (ITR), the recombinant nucleic acid molecule of claim 17 operably linked to a promoter, and an AAV 3′ ITR.

24. The recombinant nucleic acid molecule of claim 23, wherein the promoter sequence is the nucleotide sequence:
tctggcgatttccactgggcgcctcggagagcggacttcccagtgtgcatcggggcacagcgactcctggaagtggccaagggccactt ctgctaatggactccatttcccagcgctccccagatctgggcgactcagatcccagccagtggacttagcccctgtttgctcctccgataact ggggtgaccttggttaatattcaccagcagcctcccccgttgcccctctggatccactgcttaaatacggacgaggacagggccctgtctcct cagcttcaggcaccaccactgacctgggacagtgaatc (SEQ ID NO:56), or the nucleotide sequence: tctggcgatttccactgggcgcctcggagagcggacttcccagtgtgcatcggggcacagcgactcctggaagtggccaagggccactt ctgctaatggactccatttcccagcgctcccc (SEQ ID NO:54), operably linked to the nucleotide sequence: ggcgactcagatcccagccagtggacttagcccctgtttgctcctccgataactggggtgaccttggttaatattcaccagcagcctcccccg ttgcccctctggatccactgcttaaatacggacgaggacagggccctgtctcctcagcttcaggcaccaccactgacctgggacagtgaatc (SEQ ID NO:55).

25. An AAV particle comprising the recombinant nucleic acid molecule of claim 17.

26. A composition comprising the AAV particle of claim 9 in a pharmaceutically acceptable carrier.

27. The composition of claim 26, wherein the AAV particle comprises a nucleotide sequence encoding FVIIa or a derivative thereof.

28. A method of administering a nucleic acid molecule to a cell, comprising contacting the cell with the AAV particle of claim 9.

29. A method of delivering a nucleic acid molecule to a subject, comprising administering to the subject the AAV particle of claim 9.

30. A method of treating a bleeding disorder in a subject in need thereof, comprising administering to the subject the AAV particle of claim 9.

31. The method of claim 29, wherein the subject is a human.

32. The method of claim 30, wherein the bleeding disorder is hemophilia A, hemophilia B, FV deficiency, FXII deficiency, FXI deficiency, or FVII deficiency.

33. The method of claim 30, wherein the subject has, or is suspected of having, an inhibitor.

34. The method of claim 33, wherein the inhibitor is an antibody that binds factor VIII (FVIII) or factor IX (FIX).

35. (canceled)

36. A synthetic promoter comprising the nucleotide sequence:
tctggcgatttccactgggcgcctcggagctgcggacttcccagtgtgcatcggggcacagcgactcctggaagtggccaagggccactt ctgctaatggactccatttcccagcgctcccc (SEQ ID NO:54), operably linked to the nucleotide sequence: ggcgactcagatcccagccagtggacttagcccctgtttgctcctccgataactggggtgaccttggttaatattcaccagcagcctcccccg ttgcccctctggatccactgcttaaatacggacgaggacagggccctgtctcctcagcttcaggcaccaccactgacctgggacagtgaatc (SEQ ID NO:55).

37. The synthetic promoter sequence of claim 36, comprising the nucleotide sequence:
(SEQ ID NO: 56)
tctggcgatttccactgggcgcctcggagctgcggacttcccagtgtg
catcggggcacagcgactcctggaagtggccaagggccacttctgcta
atggactccatttcccagcgctccccagatctgggcgactcagatccc
agccagtggacttagcccctgtttgctcctccgataactggggtgacc
ttggttaatattcaccagcagcctcccccgttgcccctctggatccac
tgcttaaatacggacgaggacagggccctgtctcctcagcttcaggca
ccaccactgacctgggacagtgaatc.


SEQUENCE LISTING (56 sequences)

SEQ ID NO: 1 - protein, 28 aa, Artificial Sequence
  signal peptide sequence
MFPGCPRLWVLVVLGTSWVGWGSQGTEA

SEQ ID NO: 2 - protein, 709 aa, Artificial Sequence
  humanized VH chain sequence
AQLRQFYVAAQGISWSYRPEPTNSSLNLSVTSFKKIVYREYEPYFKKEKPQSTISGLLGPTLYAEVGDIIKVHFKNKADKPLSIHPQGIRYSKLSEGASYLDHTFPAEKMDDAVAPGREYTYEWSISEDSGPTHDDPPCLTHIYYSHENLIEDFNSGLIGPLLICKKGTLTEGGTQKTFDKQIVLLFAVFDESKSWSQSSSLMYTVNGYVNGTMPDITVCAHDHISWHLLGMSSGPELFSIHFNGQVLEQNHHKVSAITLVSATSTTANMTVGPEGKWIISSLTPKHLQAGMQAYIDIKNCPKKTRNLKKITREQRRHMKRWEYFIAAEEVIWDYAPVIPANMDKKYRSQHLDNFSNQIGKHYKKVMYTQYEDESFTKHTVNPNMKEDGILGPIIRAQVRDTLKIVFKNMASRPYSIYPHGVTFSPYEDEVNSSFTSGRNNTMIRAVQPGETYTYKWNILEFDEPTENDAQCLTRPYYSDVDIMRDIASGLIGLLLICKSRSLDRRGIQRAADIEQQAVFAVFDENKSWYLEDNINKFCENPDEVKRDDPKFYESNIMSTINGYVPESITTLGFCFDDTVQWHFCSVGTQNEILTIHFTGHSFIYGKRHEDTLTLFPMRGESVTVTMDNVGTWMLTSMNSSPRSKKLRLKFRDVKCIPDDDEDSYEIFEPPESTVMATRKMHDRLEPEDEESDADYDYQNRLAAALGIR

SEQ ID NO: 3 - protein, 651 aa, Artificial Sequence
  humanized VL chain sequence
SNNGNRRNYYIAAEEISWDYSEFVQRETDIEDSDDIPEDTTYKKVVFRKYLDSTFTKRDPRGEYEEHLGILGPIIRAEVDDVIQVRFKNLASRPYSLHAHGLSYEKSSEGKTYEDDSPEWFKEDNAVQPNSSYTYVWHATERSGPESPGSACRAWAYYSAVNPEKDIHSGLIGPLLICQKGILHKDSNMPMDMREFVLLFMTFDEKKSWYYEKKSRSSWRLTSSEMKKSHEFHAINGMIYSLPGLKMYEQEWVRLHLLNIGGSQDIHVVHFHGQTLLENGNKQHQLGVWPLLPGSFKTLEMKASKPGWWLLNTEVGENQRAGMQTPFLIMDRDCRMPMGLSTGIISDSQIKASEFLGYWEPRLARLNNGGSYNAWSVEKLAAEFASKPWIQVDMQKEVIITGIQTQGAKHYLKSCYTTEFYVAYSSNQINWQIFKGNSTRNVMYFNGNSDASTIKENQFDPPIVARYIRISPTRAYNRPTLRLELQGCEVNGCSTPLGMENGKIENKQITASSFKKSWWGDYWEPFRARLNAQGRVNAWQAKANNNKQWLEIDLLKIKKITAIITQGCKSLSSEMYVKSYTIHYSEQGVEWKPYRLKSSMVDKIFEGNTNTKGHVKNFFNPPIISRFIRVIPKTWNQSIALRLELFGCDIY

SEQ ID NO: 4 - protein, 836 aa, Homo sapiens
SFRNSSLNQEEEEFNLTALALENGTEFVSSNTDIIVGSNYSSPSNISKFTVNNLAEPQKAPSHQQATTAGSPLRHLIGKNSVLNSSTAEHSSPYSEDPIEDPLQPDVTGIRLLSLGAGEFKSQEHAKHKGPKVERDQAAKHRFSWMKLLAHKVGRHLSQDTGSPSGMRPWEDLPSQDTGSPSRMRPWKDPPSDLLLLKQSNSSKILVGRWHLASEKGSYEIIQDTDEDTAVNNWLISPQNASRAWGESTPLANKPGKQSGHPKFPRVRHKSLQVRQDGGKSRLKKSQFLIKTRKKKKEKHTHHAPLSPRTFHPLRSEAYNTFSERRLKHSLVLHKSNETSLPTDLNQTLPSMDFGWIASLPDHNQNSSNDTGQASCPPGLYQTVPPEEHYQTFPIQDPDQMHSTSDPSHRSSSPELSEMLEYDRSHKSFPTDISQMSPSSEHEVWQTVISPDLSQVTLSPELSQTNLSPDLSHTTLSPELIQRNLSPALGQMPISPDLSHTTLSPDLSHTTLSLDLSQTNLSPELSQTNLSPALGQMPLSPDLSHTTLSLDFSQTNLSPELSHMTLSPELSQTNLSPALGQMPISPDLSHTTLSLDFSQTNLSPELSQTNLSPALGQMPLSPDPSHTTLSLDLSQTNLSPELSQTNLSPDLSEMPLFADLSQIPLTPDLDQMTLSPDLGETDLSPNFGQMSLSPDLSQVTLSPDISDTTLLPDLSQISPPPDLDQIFYPSESSQSLLLQEFNESFPYPDLGQMPSPSSPTLNDTFLSKEFNPLVIVGLSKDGTDYIEIIPKEEVQSSEDDYAEIDYVPYDDPYKTDVRTNINSSRDPDNIAAWYLR

SEQ ID NO: 5 - DNA, 4185 bp, Artificial Sequence
  recombinant nucleic acid sequence
atgtttcctggatgtccaagactgtgggtcctggtcgtgctgggaacttcatgggtgggatggggctctcagggaaccgaggccgcacagctgcgccagttctatgtggccgcccagggcatctcttggagctaccggccagagcccaccaatagctccctgaacctgtccgtgacatctttcaagaagatcgtgtacagagagtatgagccatactttaagaaggagaagccacagagcaccatctccggcctgctgggaccaacactgtacgcagaagtgggcgacatcatcaaggtgcacttcaagaacaaggccgataagcctctgagcatccacccacagggcatccgctactctaagctgagcgagggcgcctcctatctggaccacacctttccagccgagaagatggacgatgcagtggcaccaggaagggagtacacatatgagtggtccatctctgaggacagcggaccaacccacgacgatccaccttgcctgacacacatctactattctcacgagaatctgatcgaggatttcaacagcggcctgatcggccccctgctgatctgtaagaagggcaccctgacagagggcggcacccagaagacatttgacaagcagatcgtgctgctgttcgccgtgtttgatgagagcaagtcctggagccagtctagctccctgatgtacaccgtgaatggctatgtgaacggcaccatgccagacatcacagtgtgcgcccacgatcacatctcttggcacctgctgggaatgtctagcggaccagagctgttcagcatccactttaatggccaggtgctggagcagaaccaccacaaggtgtccgccatcaccctggtgtccgccacatctaccacagccaatatgaccgtgggccccgagggcaagtggatcatctcctctctgacacctaagcacctgcaggccggcatgcaggcctacatcgacatcaagaattgtcctaagaagacccgcaacctgaagaagatcacacgggagcagcggagacacatgaagagatgggagtatttcatcgccgccgaggaagtgatctgggattacgcccctgtgatcccagccaacatggacaagaagtataggtcccagcacctggataatttctctaaccagatcggcaagcactacaagaaagtgatgtatacccagtacgaggacgagagctttaccaagcacacagtgaatcctaacatgaaggaggacggcatcctgggcccaatcatcagggcccaggtgcgcgataccctgaagatcgtgttcaagaatatggcctccaggccctattctatctaccctcacggcgtgacattctctccttacgaggatgaggtgaacagctcctttaccagcggcagaaacaatacaatgatcagggccgtgcagccaggcgagacatacacatataagtggaatatcctggagtttgacgagccaaccgagaacgatgcccagtgcctgacaagaccctactattccgatgtggacatcatgagggacatcgcctctggcctgatcggcctgctgctgatctgtaagagccgctccctggacaggaggggaatccagagggcagcagatatcgagcagcaggccgtgttcgccgtgtttgacgagaataagtcctggtacctggaggataatatcaacaagttctgcgagaaccccgatgaggtgaagagagacgatcctaagttttatgagagcaatatcatgtccaccatcaacggctacgtgccagagagcatcaccacactgggcttctgctttgacgataccgtgcagtggcacttctgttctgtgggcacacagaacgagatcctgaccatccacttcacaggccacagctttatctatggcaagcgccacgaggacaccctgacactgtttcccatgcggggcgagagcgtgaccgtgacaatggataatgtgggcacctggatgctgacaagcatgaactctagccccaggtccaagaagctgcggctgaagttcagagacgtgaagtgtatccctgacgatgacgaggattcctacgagatctttgagccacccgagtctaccgtgatggccacacgcaagatgcacgaccggctggagcccgaggatgaggagtccgatgccgactacgattatcagaacagactggccgccgccctgggaatcaggagaaagaggcgcaagaggagcaacaatggcaatcggagaaactactatatcgccgccgaggagatctcttgggactatagcgagttcgtgcagcgcgagacagacatcgaggattccgatgacatccccgaggataccacatacaagaaggtggtgttccggaagtatctggactctacctttacaaagcgggatcctagaggcgagtacgaggagcacctgggaatcctgggaccaatcatcagagccgaggtggatgacgtgatccaggtgagattcaagaacctggcctccaggccttactctctgcacgcccacggcctgtcctatgagaagtcctctgagggcaagacctacgaggatgactctcctgagtggtttaaggaggacaatgccgtgcagccaaacagctcctacacctacgtgtggcacgcaacagagagatccggaccagagagccctggatccgcctgcagggcctgggcctactatagcgccgtgaatcccgagaaggacatccactccggcctgatcggccctctgctgatctgtcagaagggcatcctgcacaaggacagcaacatgcctatggatatgagagagttcgtgctgctgttcatgacctttgatgagaagaagtcttggtactatgagaagaagagcaggtctagctggcgcctgacatcctctgagatgaagaagtcccacgagtttcacgccatcaatggcatgatctactctctgccaggcctgaagatgtatgagcaggagtgggtgaggctgcacctgctgaacatcggcggcagccaggacatccacgtggtgcacttccacggccagaccctgctggagaatggcaacaagcagcaccagctgggcgtgtggccactgctgccaggcagctttaagaccctggagatgaaggcctccaagcccggctggtggctgctgaataccgaagtgggagagaaccagagggcaggaatgcagacaccattcctgatcatggacagggattgcaggatgccaatgggcctgagcaccggaatcatctctgacagccagatcaaggcctccgagtttctgggctattgggagccccggctggccagactgaacaatggcggcagctacaatgcatggtccgtggagaagctggcagcagagttcgccagcaagccttggatccaggtggatatgcagaaggaagtgatcatcaccggcatccagacacagggcgccaagcactacctgaagtcctgttataccacagagttttatgtggcctacagctccaatcagatcaactggcagatcttcaagggcaatagcacccggaacgtgatgtactttaatggcaactctgacgccagcacaatcaaggagaaccagttcgatcctccaatcgtggccaggtatatccgcatcagccctacccgggcctacaatagaccaacactgaggctggagctgcagggctgcgaggtgaacggctgttccacccctctgggcatggagaatggcaagatcgagaacaagcagatcacagcctctagcttcaagaagtcttggtggggcgactactgggagcccttccgggcccggctgaacgcacagggaagggtgaacgcctggcaggccaaggccaacaataacaagcagtggctggagatcgatctgctgaagatcaagaagatcaccgccatcatcacacagggctgcaagtccctgtcctctgagatgtatgtgaagtcttacaccatccactatagcgagcagggcgtggagtggaagccctaccggctgaagagctccatggtggacaagatcttcgagggcaataccaacacaaagggccacgtgaagaatttctttaacccccctatcatcagccggtttatcagagtgatccctaagacttggaatcagagtattgccctgcgactggaactgtttggctgtgacatctattga

SEQ ID NO: 6 - protein, 20 aa, Artificial Sequence
  signal peptide sequence
MVSQALRLLCLLLGLQGCLA

SEQ ID NO: 7 - protein, 28 aa, Artificial Sequence
  signal peptide sequence
MQRVNMIMAESPGLITICLLGYLLSAEC

SEQ ID NO: 8 - protein, 19 aa, Artificial Sequence
  signal peptide sequence
MQIELSTCFFLCLLRFCFS

SEQ ID NO: 9 - protein, 19 aa, Artificial Sequence
  signal peptide sequence
MFSMRIVCLVLSVVGTAWT

SEQ ID NO: 10 - protein, 30 aa, Artificial Sequence
  signal peptide sequence
MKRMVSWSFHKLKTMKHLLLLLLCVFLVKS

SEQ ID NO: 11 - protein, 26 aa, Artificial Sequence
  signal peptide sequence
MSWSLHPRNLILYFYALLFLSSTCVA

SEQ ID NO: 12 - protein, 19 aa, Artificial Sequence
  signal peptide sequence
MRALLLLGFLLVSLESTLS

SEQ ID NO: 13 - protein, 18 aa, Artificial Sequence
  signal peptide sequence
MWQLTSLLLFVATWGISG

SEQ ID NO: 14 - protein, 24 aa, Artificial Sequence
  signal peptide sequence
MRVLGGRCGALLACLLLVLPVSEA

SEQ ID NO: 15 - protein, 24 aa, Artificial Sequence
  signal peptide sequence
MAHVRGLQLPGCLALAALCSLVHS

SEQ ID NO: 16 - protein, 32 aa, Artificial Sequence
  signal peptide sequence
MYSNVIGTVTSGKRKVYLLSLLLIGFWDCVTC

SEQ ID NO: 17 - protein, 18 aa, Artificial Sequence
  signal peptide sequence
MKWVTFISLLFLFSSAYS

SEQ ID NO: 18 - protein, 19 aa, Artificial Sequence
  signal peptide sequence
MRLAVGALLVCAVLGLCLA

SEQ ID NO: 19 - protein, 24 aa, Artificial Sequence
  signal peptide sequence
MPSSVSWGILLLAGLCCLVPVSLA

SEQ ID NO: 20 - protein, 31 aa, Artificial Sequence
  signal peptide sequence
MLRGPGPGLLLLAVQCLGTAVPSTGASKSKR

SEQ ID NO: 21 - protein, 19 aa, Artificial Sequence
  signal peptide sequence
MRSLGALLLLLSACLAVSA

SEQ ID NO: 22 - protein, 23 aa, Artificial Sequence
  signal peptide sequence
MERMLPLLALGLLAAGFCPAVLC

SEQ ID NO: 23 - protein, 18 aa, Artificial Sequence
  signal peptide sequence
MKAAVLTLAVLFLTGSQA

SEQ ID NO: 24 - protein, 27 aa, Artificial Sequence
  signal peptide sequence
MDPPRPALLALLALPALLLLLLAGARA

SEQ ID NO: 25 - protein, 18 aa, Artificial Sequence
  signal peptide sequence
MKVLWAALLVTFLAGCQA

SEQ ID NO: 26 - protein, 18 aa, Artificial Sequence
  signal peptide sequence
MKWVESIFLIFLLNFTES

SEQ ID NO: 27 - protein, 18 aa, Artificial Sequence
  signal peptide sequence
MEKLLCFLVLTSLSHAFG

SEQ ID NO: 28 - protein, 19 aa, Artificial Sequence
  signal peptide sequence
MEHKEVVLLLLLFLKSGQG

SEQ ID NO: 29 - protein, 19 aa, Artificial Sequence
  signal peptide sequence
MKILILGIFLFLCSTPAWA

SEQ ID NO: 30 - protein, 22 aa, Artificial Sequence
  signal peptide sequence
MEGPRGWLVLCVLAISLASMVT

SEQ ID NO: 31 - protein, 20 aa, Artificial Sequence
  signal peptide sequence
MGPLMVLFCLLFLYPGLADS

SEQ ID NO: 32 - protein, 22 aa, Artificial Sequence
  signal peptide sequence
MGPTSGPSLLLLLLTHLPLALG

SEQ ID NO: 33 - protein, 19 aa, Artificial Sequence
  signal peptide sequence
MRLLWGLIWASSFFTLSLQ

SEQ ID NO: 34 - protein, 18 aa, Artificial Sequence
  signal peptide sequence
MGLLGILCFLIFLGKTWG

SEQ ID NO: 35 - protein, 21 aa, Artificial Sequence
  signal peptide sequence
MARRSVLYFILLNALINKGQA

SEQ ID NO: 36 - protein, 22 aa, Artificial Sequence
  signal peptide sequence
MKVISLFILVGFIGEFQSFSSA

SEQ ID NO: 37 - protein, 20 aa, Artificial Sequence
  signal peptide sequence
MFAVVFFILSLMTCQPGVTA

SEQ ID NO: 38 - protein, 21 aa, Artificial Sequence
  signal peptide sequence
MSACRSFAVAICILEISILTA

SEQ ID NO: 39 - protein, 27 aa, Artificial Sequence
  signal peptide sequence
MALLWGLLVLSWSCLQGPCSVFSPVSA

SEQ ID NO: 40 - protein, 22 aa, Artificial Sequence
  signal peptide sequence
MPLLLYTCLLWLPTSGLWTVQA

SEQ ID NO: 41 - protein, 18 aa, Artificial Sequence
  signal peptide sequence
MSALGAVIALLLWGQLFA

SEQ ID NO: 42 - protein, 23 aa, Artificial Sequence
  signal peptide sequence
MARVLGAPVALGLWSLCWSLAIA

SEQ ID NO: 43 - protein, 25 aa, Artificial Sequence
  signal peptide sequence
MSEVPVARVWLVLLLLTVQVGVTAG

SEQ ID NO: 44 - protein, 20 aa, Artificial Sequence
  signal peptide sequence
MASHRLLLLCLAGLVFVSEA

SEQ ID NO: 45 - protein, 21 aa, Artificial Sequence
  signal peptide sequence
MGKISSLPTQLFKCCFCDFLK

SEQ ID NO: 46 - protein, 21 aa, Artificial Sequence
  signal peptide sequence
MELTELLLVVMLLLTARLTLS

SEQ ID NO: 47 - protein, 20 aa, Artificial Sequence
  signal peptide sequence
MSRSVALAVLALLSLSGLEA

SEQ ID NO: 48 - protein, 23 aa, Artificial Sequence
  signal peptide sequence
MGKNKLLHPSLVLLLLVLLPTDA

SEQ ID NO: 49 - protein, 6 aa, Artificial Sequence
  linker sequence
RKRRKR

SEQ ID NO: 50 - DNA, 4185 bp, Artificial Sequence
  nucleotide sequence
atgttcccaggctgcccacgcctctgggtcctggtggtcttgggcaccagctgggtaggctgggggagccaagggacagaagcggcacagctaaggcagttctacgtggctgctcagggcatcagttggagctaccgacctgagcccacaaactcaagtttgaatctttctgtaacttcctttaagaaaattgtctacagagagtatgaaccatattttaagaaagaaaaaccacaatctaccatttcaggacttcttgggcctactttatatgctgaagtcggagacatcataaaagttcactttaaaaataaggcagataagcccttgagcatccatcctcaaggaattaggtacagtaaattatcagaaggtgcttcttaccttgaccacacattccctgcggagaagatggacgacgctgtggctccaggccgagaatacacctatgaatggagtatcagtgaggacagtggacccacccatgatgaccctccatgcctcacacacatctattactcccatgaaaatctgatcgaggatttcaactcggggctgattgggcccctgcttatctgtaaaaaagggaccctaactgagggtgggacacagaagacgtttgacaagcaaatcgtgctactatttgctgtgtttgatgaaagcaagagctggagccagtcatcatccctaatgtacacagtcaatggatatgtgaatgggacaatgccagatataacagtttgtgcccatgaccacatcagctggcatctgctgggaatgagctcggggccagaattattctccattcatttcaacggccaggtcctggagcagaaccatcataaggtctcagccatcacccttgtcagtgctacatccactaccgcaaatatgactgtgggcccagagggaaagtggatcatatcttctctcaccccaaaacatttgcaagctgggatgcaggcttacattgacattaaaaactgcccaaagaaaaccaggaatcttaagaaaataactcgtgagcagaggcggcacatgaagaggtgggaatacttcattgctgcagaggaagtcatttgggactatgcacctgtaataccagcgaatatggacaaaaaatacaggtctcagcatttggataatttctcaaaccaaattggaaaacattataagaaagttatgtacacacagtacgaagatgagtccttcaccaaacatacagtgaatcccaatatgaaagaagatgggattttgggtcctattatcagagcccaggtcagagacacactcaaaatcgtgttcaaaaatatggccagccgcccctatagcatttaccctcatggagtgaccttctcgccttatgaagatgaagtcaactcttctttcacctcaggcaggaacaacaccatgatcagagcagttcaaccaggggaaacctatacttataagtggaacatcttagagtttgatgaacccacagaaaatgatgcccagtgcttaacaagaccatactacagtgacgtggacatcatgagagacatcgcctctgggctaataggactacttctaatctgtaagagcagatccctggacaggcgaggaatacagagggcagcagacatcgaacagcaggctgtgtttgctgtgtttgatgagaacaaaagctggtaccttgaggacaacatcaacaagttttgtgaaaatcctgatgaggtgaaacgtgatgaccccaagttttatgaatcaaacatcatgagcactatcaatggctatgtgcctgagagcataactactcttggattctgctttgatgacactgtccagtggcacttctgtagtgtggggacccagaatgaaattttgaccatccacttcactgggcactcattcatctatggaaagaggcatgaggacaccttgaccctcttccccatgcgtggagaatctgtgacggtcacaatggataatgttggaacttggatgttaacttccatgaattctagtccaagaagcaaaaagctgaggctgaaattcagggatgttaaatgtatcccagatgatgatgaagactcatatgagatttttgaacctccagaatctacagtcatggctacacggaaaatgcatgatcgtttagaacctgaagatgaagagagtgatgctgactatgattaccagaacagactggctgcagcattaggaatcaggagaaagagaagaaagagaagcaacaatggaaacagaagaaattattacattgctgctgaagaaatatcctgggattattcagaatttgtacaaagggaaacagatattgaagactctgatgatattccagaagataccacatataagaaagtagtttttcgaaagtacctcgacagcacttttaccaaacgtgatcctcgaggggagtatgaagagcatctcggaattcttggtcctattatcagagctgaagtggatgatgttatccaagttcgttttaaaaatttagcatccagaccgtattctctacatgcccatggactttcctatgaaaaatcatcagagggaaagacttatgaagatgactctcctgaatggtttaaggaagataatgctgttcagccaaatagcagttatacctacgtatggcatgccactgagcgatcagggccagaaagtcctggctctgcctgtcgggcttgggcctactactcagctgtgaacccagaaaaagatattcactcaggcttgataggtcccctcctaatctgccaaaaaggaatactacataaggacagcaacatgcctatggacatgagagaatttgtcttactatttatgacctttgatgaaaagaagagctggtactatgaaaagaagtcccgaagttcttggagactcacatcctcagaaatgaaaaaatcccatgagtttcacgccattaatgggatgatctacagcttgcctggcctgaaaatgtatgagcaagagtgggtgaggttacacctgctgaacataggcggctcccaagacattcacgtggttcactttcacggccagaccttgctggaaaatggcaataaacagcaccagttaggggtctggccccttctgcctggttcatttaaaactcttgaaatgaaggcatcaaaacctggctggtggctcctaaacacagaggttggagaaaaccagagagcagggatgcaaacgccatttcttatcatggacagagactgtaggatgccaatgggactaagcactggtatcatatctgattcacagatcaaggcttcagagtttctgggttactgggagcccagattagcaagattaaacaatggtggatcttataatgcttggagtgtagaaaaacttgcagcagaatttgcctctaaaccttggatccaggtggacatgcaaaaggaagtcataatcacagggatccagacccaaggtgccaaacactacctgaagtcctgctataccacagagttctatgtagcttacagttccaaccagatcaactggcagatcttcaaagggaacagcacaaggaatgtgatgtattttaatggcaattcagatgcctctacaataaaagagaatcagtttgacccacctattgtggctagatatattaggatctctccaactcgagcctataacagacctacccttcgattggaactgcaaggttgtgaggtaaatggatgttccacacccctgggtatggaaaatggaaagatagaaaacaagcaaatcacagcttcttcgtttaagaaatcttggtggggagattactgggaacccttccgtgcccgtctgaatgcccagggacgtgtgaatgcctggcaagccaaggcaaacaacaataagcagtggctagaaattgatctactcaagatcaagaagataacggcaattataacacagggctgcaagtctctgtcctctgaaatgtatgtaaagagctataccatccactacagtgagcagggagtggaatggaaaccatacaggctgaaatcctccatggtggacaagatttttgaaggaaatactaataccaaaggacatgtgaagaactttttcaaccccccaatcatttccaggtttatccgtgtcattcctaaaacatggaatcaaagtattgcacttcgcctggaactctttggctgtgatatttactag

SEQ ID NO: 51 - DNA, 4167 bp, Artificial Sequence
  recombinent nucleic acid sequence
atgttcccaggctgcccacgcctctgggtcctggtggtcttgggcaccagctgggtaggctgggggagccaagggacagaagcggcacagctaaggcagttctacgtggctgctcagggcatcagttggagctaccgacctgagcccacaaactcaagtttgaatctttctgtaacttcctttaagaaaattgtctacagagagtatgaaccatattttaagaaagaaaaaccacaatctaccatttcaggacttcttgggcctactttatatgctgaagtcggagacatcataaaagttcactttaaaaataaggcagataagcccttgagcatccatcctcaaggaattaggtacagtaaattatcagaaggtgcttcttaccttgaccacacattccctgcggagaagatggacgacgctgtggctccaggccgagaatacacctatgaatggagtatcagtgaggacagtggacccacccatgatgaccctccatgcctcacacacatctattactcccatgaaaatctgatcgaggatttcaactcggggctgattgggcccctgcttatctgtaaaaaagggaccctaactgagggtgggacacagaagacgtttgacaagcaaatcgtgctactatttgctgtgtttgatgaaagcaagagctggagccagtcatcatccctaatgtacacagtcaatggatatgtgaatgggacaatgccagatataacagtttgtgcccatgaccacatcagctggcatctgctgggaatgagctcggggccagaattattctccattcatttcaacggccaggtcctggagcagaaccatcataaggtctcagccatcacccttgtcagtgctacatccactaccgcaaatatgactgtgggcccagagggaaagtggatcatatcttctctcaccccaaaacatttgcaagctgggatgcaggcttacattgacattaaaaactgcccaaagaaaaccaggaatcttaagaaaataactcgtgagcagaggcggcacatgaagaggtgggaatacttcattgctgcagaggaagtcatttgggactatgcacctgtaataccagcgaatatggacaaaaaatacaggtctcagcatttggataatttctcaaaccaaattggaaaacattataagaaagttatgtacacacagtacgaagatgagtccttcaccaaacatacagtgaatcccaatatgaaagaagatgggattttgggtcctattatcagagcccaggtcagagacacactcaaaatcgtgttcaaaaatatggccagccgcccctatagcatttaccctcatggagtgaccttctcgccttatgaagatgaagtcaactcttctttcacctcaggcaggaacaacaccatgatcagagcagttcaaccaggggaaacctatacttataagtggaacatcttagagtttgatgaacccacagaaaatgatgcccagtgcttaacaagaccatactacagtgacgtggacatcatgagagacatcgcctctgggctaataggactacttctaatctgtaagagcagatccctggacaggcgaggaatacagagggcagcagacatcgaacagcaggctgtgtttgctgtgtttgatgagaacaaaagctggtaccttgaggacaacatcaacaagttttgtgaaaatcctgatgaggtgaaacgtgatgaccccaagttttatgaatcaaacatcatgagcactatcaatggctatgtgcctgagagcataactactcttggattctgctttgatgacactgtccagtggcacttctgtagtgtggggacccagaatgaaattttgaccatccacttcactgggcactcattcatctatggaaagaggcatgaggacaccttgaccctcttccccatgcgtggagaatctgtgacggtcacaatggataatgttggaacttggatgttaacttccatgaattctagtccaagaagcaaaaagctgaggctgaaattcagggatgttaaatgtatcccagatgatgatgaagactcatatgagatttttgaacctccagaatctacagtcatggctacacggaaaatgcatgatcgtttagaacctgaagatgaagagagtgatgctgactatgattaccagaacagactggctgcagcattaggaatcaggagcaacaatggaaacagaagaaattattacattgctgctgaagaaatatcctgggattattcagaatttgtacaaagggaaacagatattgaagactctgatgatattccagaagataccacatataagaaagtagtttttcgaaagtacctcgacagcacttttaccaaacgtgatcctcgaggggagtatgaagagcatctcggaattcttggtcctattatcagagctgaagtggatgatgttatccaagttcgttttaaaaatttagcatccagaccgtattctctacatgcccatggactttcctatgaaaaatcatcagagggaaagacttatgaagatgactctcctgaatggtttaaggaagataatgctgttcagccaaatagcagttatacctacgtatggcatgccactgagcgatcagggccagaaagtcctggctctgcctgtcgggcttgggcctactactcagctgtgaacccagaaaaagatattcactcaggcttgataggtcccctcctaatctgccaaaaaggaatactacataaggacagcaacatgcctatggacatgagagaatttgtcttactatttatgacctttgatgaaaagaagagctggtactatgaaaagaagtcccgaagttcttggagactcacatcctcagaaatgaaaaaatcccatgagtttcacgccattaatgggatgatctacagcttgcctggcctgaaaatgtatgagcaagagtgggtgaggttacacctgctgaacataggcggctcccaagacattcacgtggttcactttcacggccagaccttgctggaaaatggcaataaacagcaccagttaggggtctggccccttctgcctggttcatttaaaactcttgaaatgaaggcatcaaaacctggctggtggctcctaaacacagaggttggagaaaaccagagagcagggatgcaaacgccatttcttatcatggacagagactgtaggatgccaatgggactaagcactggtatcatatctgattcacagatcaaggcttcagagtttctgggttactgggagcccagattagcaagattaaacaatggtggatcttataatgcttggagtgtagaaaaacttgcagcagaatttgcctctaaaccttggatccaggtggacatgcaaaaggaagtcataatcacagggatccagacccaaggtgccaaacactacctgaagtcctgctataccacagagttctatgtagcttacagttccaaccagatcaactggcagatcttcaaagggaacagcacaaggaatgtgatgtattttaatggcaattcagatgcctctacaataaaagagaatcagtttgacccacctattgtggctagatatattaggatctctccaactcgagcctataacagacctacccttcgattggaactgcaaggttgtgaggtaaatggatgttccacacccctgggtatggaaaatggaaagatagaaaacaagcaaatcacagcttcttcgtttaagaaatcttggtggggagattactgggaacccttccgtgcccgtctgaatgcccagggacgtgtgaatgcctggcaagccaaggcaaacaacaataagcagtggctagaaattgatctactcaagatcaagaagataacggcaattataacacagggctgcaagtctctgtcctctgaaatgtatgtaaagagctataccatccactacagtgagcagggagtggaatggaaaccatacaggctgaaatcctccatggtggacaagatttttgaaggaaatactaataccaaaggacatgtgaagaactttttcaaccccccaatcatttccaggtttatccgtgtcattcctaaaacatggaatcaaagtattgcacttcgcctggaactctttggctgtgatatttactag

SEQ ID NO: 52 - DNA, 4632 bp, Artificial Sequence
  recombinent nucleic acid sequence
atgttcccaggctgcccacgcctctgggtcctggtggtcttgggcaccagctgggtaggctgggggagccaagggacagaagcggcacagctaaggcagttctacgtggctgctcagggcatcagttggagctaccgacctgagcccacaaactcaagtttgaatctttctgtaacttcctttaagaaaattgtctacagagagtatgaaccatattttaagaaagaaaaaccacaatctaccatttcaggacttcttgggcctactttatatgctgaagtcggagacatcataaaagttcactttaaaaataaggcagataagcccttgagcatccatcctcaaggaattaggtacagtaaattatcagaaggtgcttcttaccttgaccacacattccctgcggagaagatggacgacgctgtggctccaggccgagaatacacctatgaatggagtatcagtgaggacagtggacccacccatgatgaccctccatgcctcacacacatctattactcccatgaaaatctgatcgaggatttcaactcggggctgattgggcccctgcttatctgtaaaaaagggaccctaactgagggtgggacacagaagacgtttgacaagcaaatcgtgctactatttgctgtgtttgatgaaagcaagagctggagccagtcatcatccctaatgtacacagtcaatggatatgtgaatgggacaatgccagatataacagtttgtgcccatgaccacatcagctggcatctgctgggaatgagctcggggccagaattattctccattcatttcaacggccaggtcctggagcagaaccatcataaggtctcagccatcacccttgtcagtgctacatccactaccgcaaatatgactgtgggcccagagggaaagtggatcatatcttctctcaccccaaaacatttgcaagctgggatgcaggcttacattgacattaaaaactgcccaaagaaaaccaggaatcttaagaaaataactcgtgagcagaggcggcacatgaagaggtgggaatacttcattgctgcagaggaagtcatttgggactatgcacctgtaataccagcgaatatggacaaaaaatacaggtctcagcatttggataatttctcaaaccaaattggaaaacattataagaaagttatgtacacacagtacgaagatgagtccttcaccaaacatacagtgaatcccaatatgaaagaagatgggattttgggtcctattatcagagcccaggtcagagacacactcaaaatcgtgttcaaaaatatggccagccgcccctatagcatttaccctcatggagtgaccttctcgccttatgaagatgaagtcaactcttctttcacctcaggcaggaacaacaccatgatcagagcagttcaaccaggggaaacctatacttataagtggaacatcttagagtttgatgaacccacagaaaatgatgcccagtgcttaacaagaccatactacagtgacgtggacatcatgagagacatcgcctctgggctaataggactacttctaatctgtaagagcagatccctggacaggcgaggaatacagagggcagcagacatcgaacagcaggctgtgtttgctgtgtttgatgagaacaaaagctggtaccttgaggacaacatcaacaagttttgtgaaaatcctgatgaggtgaaacgtgatgaccccaagttttatgaatcaaacatcatgagcactatcaatggctatgtgcctgagagcataactactcttggattctgctttgatgacactgtccagtggcacttctgtagtgtggggacccagaatgaaattttgaccatccacttcactgggcactcattcatctatggaaagaggcatgaggacaccttgaccctcttccccatgcgtggagaatctgtgacggtcacaatggataatgttggaacttggatgttaacttccatgaattctagtccaagaagcaaaaagctgaggctgaaattcagggatgttaaatgtatcccagatgatgatgaagactcatatgagatttttgaacctccagaatctacagtcatggctacacggaaaatgcatgatcgtttagaacctgaagatgaagagagtgatgctgactatgattaccagaacagactggctgcagcattaggaatcaggtcattccgaaactcatcattgaatcaggaagaagaagagttcaatcttactgccctagctctggagaatggcactgaattcgtttcttcaaacacagatataattgttggttcaaattattcttccccaagtaatattagtaagttcactgtcaataaccttgcagaacctcagaaagccccttctcaccaacaagccaccacagctggttccccactgagacacctcattggcaagaactcagttctcaattcttccacagcagagcattccagcccatattctgaagaccctatagaggatacagattacattgagatcattccaaaggaagaggtccagagcagtgaagatgactatgctgaaattgattatgtgccctatgatgacccctacaaaactgatgttaggacaaacatcaactcctccagagatcctgacaacattgcagcatggtacctccgcagcaacaatggaaacagaagaaattattacattgctgctgaagaaatatcctgggattattcagaatttgtacaaagggaaacagatattgaagactctgatgatattccagaagataccacatataagaaagtagtttttcgaaagtacctcgacagcacttttaccaaacgtgatcctcgaggggagtatgaagagcatctcggaattcttggtcctattatcagagctgaagtggatgatgttatccaagttcgttttaaaaatttagcatccagaccgtattctctacatgcccatggactttcctatgaaaaatcatcagagggaaagacttatgaagatgactctcctgaatggtttaaggaagataatgctgttcagccaaatagcagttatacctacgtatggcatgccactgagcgatcagggccagaaagtcctggctctgcctgtcgggcttgggcctactactcagctgtgaacccagaaaaagatattcactcaggcttgataggtcccctcctaatctgccaaaaaggaatactacataaggacagcaacatgcctatggacatgagagaatttgtcttactatttatgacctttgatgaaaagaagagctggtactatgaaaagaagtcccgaagttcttggagactcacatcctcagaaatgaaaaaatcccatgagtttcacgccattaatgggatgatctacagcttgcctggcctgaaaatgtatgagcaagagtgggtgaggttacacctgctgaacataggcggctcccaagacattcacgtggttcactttcacggccagaccttgctggaaaatggcaataaacagcaccagttaggggtctggccccttctgcctggttcatttaaaactcttgaaatgaaggcatcaaaacctggctggtggctcctaaacacagaggttggagaaaaccagagagcagggatgcaaacgccatttcttatcatggacagagactgtaggatgccaatgggactaagcactggtatcatatctgattcacagatcaaggcttcagagtttctgggttactgggagcccagattagcaagattaaacaatggtggatcttataatgcttggagtgtagaaaaacttgcagcagaatttgcctctaaaccttggatccaggtggacatgcaaaaggaagtcataatcacagggatccagacccaaggtgccaaacactacctgaagtcctgctataccacagagttctatgtagcttacagttccaaccagatcaactggcagatcttcaaagggaacagcacaaggaatgtgatgtattttaatggcaattcagatgcctctacaataaaagagaatcagtttgacccacctattgtggctagatatattaggatctctccaactcgagcctataacagacctacccttcgattggaactgcaaggttgtgaggtaaatggatgttccacacccctgggtatggaaaatggaaagatagaaaacaagcaaatcacagcttcttcgtttaagaaatcttggtggggagattactgggaacccttccgtgcccgtctgaatgcccagggacgtgtgaatgcctggcaagccaaggcaaacaacaataagcagtggctagaaattgatctactcaagatcaagaagataacggcaattataacacagggctgcaagtctctgtcctctgaaatgtatgtaaagagctataccatccactacagtgagcagggagtggaatggaaaccatacaggctgaaatcctccatggtggacaagatttttgaaggaaatactaataccaaaggacatgtgaagaactttttcaaccccccaatcatttccaggtttatccgtgtcattcctaaaacatggaatcaaagtattgcacttcgcctggaactctttggctgtgatatttactag

SEQ ID NO: 53 - DNA, 4209 bp, Artificial Sequence
  recombinent nucleic acid sequence
atgttcccaggctgcccacgcctctgggtcctggtggtcttgggcaccagctgggtaggctgggggagccaagggacagaagcggcacagctaaggcagttctacgtggctgctcagggcatcagttggagctaccgacctgagcccacaaactcaagtttgaatctttctgtaacttcctttaagaaaattgtctacagagagtatgaaccatattttaagaaagaaaaaccacaatctaccatttcaggacttcttgggcctactttatatgctgaagtcggagacatcataaaagttcactttaaaaataaggcagataagcccttgagcatccatcctcaaggaattaggtacagtaaattatcagaaggtgcttcttaccttgaccacacattccctgcggagaagatggacgacgctgtggctccaggccgagaatacacctatgaatggagtatcagtgaggacagtggacccacccatgatgaccctccatgcctcacacacatctattactcccatgaaaatctgatcgaggatttcaactcggggctgattgggcccctgcttatctgtaaaaaagggaccctaactgagggtgggacacagaagacgtttgacaagcaaatcgtgctactatttgctgtgtttgatgaaagcaagagctggagccagtcatcatccctaatgtacacagtcaatggatatgtgaatgggacaatgccagatataacagtttgtgcccatgaccacatcagctggcatctgctgggaatgagctcggggccagaattattctccattcatttcaacggccaggtcctggagcagaaccatcataaggtctcagccatcacccttgtcagtgctacatccactaccgcaaatatgactgtgggcccagagggaaagtggatcatatcttctctcaccccaaaacatttgcaagctgggatgcaggcttacattgacattaaaaactgcccaaagaaaaccaggaatcttaagaaaataactcgtgagcagaggcggcacatgaagaggtgggaatacttcattgctgcagaggaagtcatttgggactatgcacctgtaataccagcgaatatggacaaaaaatacaggtctcagcatttggataatttctcaaaccaaattggaaaacattataagaaagttatgtacacacagtacgaagatgagtccttcaccaaacatacagtgaatcccaatatgaaagaagatgggattttgggtcctattatcagagcccaggtcagagacacactcaaaatcgtgttcaaaaatatggccagccgcccctatagcatttaccctcatggagtgaccttctcgccttatgaagatgaagtcaactcttctttcacctcaggcaggaacaacaccatgatcagagcagttcaaccaggggaaacctatacttataagtggaacatcttagagtttgatgaacccacagaaaatgatgcccagtgcttaacaagaccatactacagtgacgtggacatcatgagagacatcgcctctgggctaataggactacttctaatctgtaagagcagatccctggacaggcgaggaatacagagggcagcagacatcgaacagcaggctgtgtttgctgtgtttgatgagaacaaaagctggtaccttgaggacaacatcaacaagttttgtgaaaatcctgatgaggtgaaacgtgatgaccccaagttttatgaatcaaacatcatgagcactatcaatggctatgtgcctgagagcataactactcttggattctgctttgatgacactgtccagtggcacttctgtagtgtggggacccagaatgaaattttgaccatccacttcactgggcactcattcatctatggaaagaggcatgaggacaccttgaccctcttccccatgcgtggagaatctgtgacggtcacaatggataatgttggaacttggatgttaacttccatgaattctagtccaagaagcaaaaagctgaggctgaaattcagggatgttaaatgtatcccagatgatgatgaagactcatatgagatttttgaacctccagaatctacagtcatggctacacggaaaatgcatgatcgtttagaacctgaagatgaagagagtgatgctgactatgattaccagaacagactggctgcagcattaggaatcaggtcattccgaaaccctgacaacattgcagcatggtacctccgcagcaacaatggaaacagaagaaattattacattgctgctgaagaaatatcctgggattattcagaatttgtacaaagggaaacagatattgaagactctgatgatattccagaagataccacatataagaaagtagtttttcgaaagtacctcgacagcacttttaccaaacgtgatcctcgaggggagtatgaagagcatctcggaattcttggtcctattatcagagctgaagtggatgatgttatccaagttcgttttaaaaatttagcatccagaccgtattctctacatgcccatggactttcctatgaaaaatcatcagagggaaagacttatgaagatgactctcctgaatggtttaaggaagataatgctgttcagccaaatagcagttatacctacgtatggcatgccactgagcgatcagggccagaaagtcctggctctgcctgtcgggcttgggcctactactcagctgtgaacccagaaaaagatattcactcaggcttgataggtcccctcctaatctgccaaaaaggaatactacataaggacagcaacatgcctatggacatgagagaatttgtcttactatttatgacctttgatgaaaagaagagctggtactatgaaaagaagtcccgaagttcttggagactcacatcctcagaaatgaaaaaatcccatgagtttcacgccattaatgggatgatctacagcttgcctggcctgaaaatgtatgagcaagagtgggtgaggttacacctgctgaacataggcggctcccaagacattcacgtggttcactttcacggccagaccttgctggaaaatggcaataaacagcaccagttaggggtctggccccttctgcctggttcatttaaaactcttgaaatgaaggcatcaaaacctggctggtggctcctaaacacagaggttggagaaaaccagagagcagggatgcaaacgccatttcttatcatggacagagactgtaggatgccaatgggactaagcactggtatcatatctgattcacagatcaaggcttcagagtttctgggttactgggagcccagattagcaagattaaacaatggtggatcttataatgcttggagtgtagaaaaacttgcagcagaatttgcctctaaaccttggatccaggtggacatgcaaaaggaagtcataatcacagggatccagacccaaggtgccaaacactacctgaagtcctgctataccacagagttctatgtagcttacagttccaaccagatcaactggcagatcttcaaagggaacagcacaaggaatgtgatgtattttaatggcaattcagatgcctctacaataaaagagaatcagtttgacccacctattgtggctagatatattaggatctctccaactcgagcctataacagacctacccttcgattggaactgcaaggttgtgaggtaaatggatgttccacacccctgggtatggaaaatggaaagatagaaaacaagcaaatcacagcttcttcgtttaagaaatcttggtggggagattactgggaacccttccgtgcccgtctgaatgcccagggacgtgtgaatgcctggcaagccaaggcaaacaacaataagcagtggctagaaattgatctactcaagatcaagaagataacggcaattataacacagggctgcaagtctctgtcctctgaaatgtatgtaaagagctataccatccactacagtgagcagggagtggaatggaaaccatacaggctgaaatcctccatggtggacaagatttttgaaggaaatactaataccaaaggacatgtgaagaactttttcaaccccccaatcatttccaggtttatccgtgtcattcctaaaacatggaatcaaagtattgcacttcgcctggaactctttggctgtgatatttactag

SEQ ID NO: 54 - DNA, 122 bp, Artificial
  Chr19
tctggcgatttccactgggcgcctcggagctgcggacttcccagtgtgcatcggggcacagcgactcctggaagtggccaagggccacttctgctaatggactccatttcccagcgctcccc

SEQ ID NO: 55 - DNA, 185 bp, Artificial
  AIAT
ggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatc

SEQ ID NO: 56 - DNA, 314 bp, Artificial
  Ch19-AIAT
tctggcgatttccactgggcgcctcggagctgcggacttcccagtgtgcatcggggcacagcgactcctggaagtggccaagggccacttctgctaatggactccatttcccagcgctccccagatctgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatc